(19) United States
(12) Patent Application Publication
(10) Pub. No.: US 2025/0367276 A1
(43) Pub. Date: Dec. 4, 2025

(54) MICROBIOTA ANALOGS OF TUMOR ASSOCIATED ANTIGENS AND USES THEREOF

(71) Applicant: ISTITUTO NAZIONALE TUMORI IRCCS - FONDAZIONE G. PASCALE, Napoli (IT)

(72) Inventors: Luigi BUONAGURO, Napoli (IT); Maria TAGLIAMONTE, Napoli (IT); Angela MAURIELLO, Napoli (IT); Beatrice CAVALLUZZO, Napoli (IT); Concetta RAGONE, Napoli (IT); Carmen MANOLIO, Napoli (IT)

(73) Assignee: ISTITUTO NAZIONALE TUMORI IRCCS - FONDAZIONE G. PASCALE, Napoli (IT)

(21) Appl. No.: 18/854,096

(22) PCT Filed: Apr. 6, 2023
(86) PCT No.: PCT/EP2023/059189

§ 371 Date: Oct. 4, 2024

(30) Foreign Application Priority Data
Apr. 6, 2022 (IT) 102022000006785

(51) Int. Cl. A61K 39/02; A61K 38/16; A61K 39/00

(52) U.S. Cl. CPC A61K 39/0216; A61K 38/164; A61K 39/02; A61K 2039/585

(57) ABSTRACT

The present invention relates to tumor antigens and their use in cancer immunotherapy. In particular, the present invention relates to the identifications of microbiota sequence analogs of tumor associated antigenic epitope sequences which can be used in tumor vaccination therapies in the treatment and prevention of cancer and have a significant impact on cancer progression and prognosis.

### TECHNICAL FIELD

The present invention relates to tumor antigens and their use in cancer immunotherapy. In particular, the present invention relates to the identifications of microbiota sequence analogs of tumor associated antigenic epitope sequences which can be used in tumor vaccination therapies in the treatment and prevention of cancer and have a significant impact on cancer progression and prognosis.

### BACKGROUND

The gut microbiota profile is unique for each individual and is composed by different bacteria species according to individual birth-to-infant transitions. Although the healthy core native microbiota remains relatively stable in adulthood, they are significantly influenced during the lifetime by several individual factors.

In the last years, the local and systemic effects of microbiota on cancer onset, progression and response to treatments, such as immunotherapies, has been extensively described. Several mechanisms have been elucidated to explain such effects.

The present invention provides a new perspective, proposing a role of microbiota based on the molecular mimicry of tumor associated antigens by microbiome-associated antigens. Indeed, this may shape the T cell immunity with a significant impact on anti-cancer immunity and, consequently, on cancer progression and prognosis.

The human gastrointestinal tract is colonized by approximately 1014 microbes equating to roughly 1,000 times the number of cells and 10,000 times the DNA content of the human body (1). Gut microbiota are composed of several species of microorganisms, including bacteria, yeast, and viruses. However, only a few phyla are represented, accounting for more than 160 species with the two phyla Firmicutes and Bacteroidetes accounting for 90% of gut microbiota (2). Human gut microbiota show inter-individual and intra-individual variations due to infant transitions, age and environmental factors including antibiotic use. Birth gestational age represents the major determinant of gut microbiota composition with a great impact on maturation of systemic immunity (3, 4). In addition, type of delivery (5), methods of milk feeding (6) and weaning period (7) have a major impact on the microbiota composition in the first year of life. Diversity increases during the following months and at approximately three years of age, a child's gut microbiota composition and diversity are most like those of adults, according to the influence of genetics, environment, diet, lifestyle, and gut physiology (8). Once the core of the individual gut microbiota composition is established, variations can occur during the life due to several factors, including enterotypes, body mass index (BMI) level, lifestyle, exercise frequency, ethnicity, dietary and cultural habits (9).

Gut bacteria are master regulators of digestion along the gastrointestinal tract and prevent bacteria invasion by maintaining the intestinal epithelium integrity (10). In addition, commensal bacteria and their products have been shown to regulate the development, homeostasis, and function of innate and adaptive immune cells via nutrient- and metabolite-dependent mechanisms (11). Besides the physiological role, the microbiota has been shown to have a role in human diseases (12, 13) and to influence both tumor development and treatment response (14). In particular, specific microbiota populations have been show to affect cancer genesis and development, either in a positive or in a negative way, depending on its own composition. This is a consequence of the production of metabolites which may impact on tumor cells and/or on anti-tumor immune response (15). In addition, while components such as LPS or MPL are able to activate immune T cell-mediated response against cancer cells (16), Natural Killers can be inhibited by the bacterial virulence factor Fap2 (17). Moreover, development of Tregs can be driven by polysaccharide A and TLR signaling to dendritic cells (18, 19) and Th17 can be induced by specific bacterial subsets such as segmented filamentous bacteria (SFB) (20).

In addition to such “generic” mechanisms of modulation, the impact of the gut microbiota on the anti-cancer immune response can be driven by a “molecular mimicry” between bacteria and tumor associated antigens. The gut microbiome encodes over 3 million genes as whole, whereas the entire human genome consists of approximately 23,000 genes (21). This implies a high probability of nucleic and protein sequences with homology between microbiota populations and cellular antigens. This may result in an overlapping peptidome representation which may be targeted by cross-reactive CD8+ T cell receptors (TCRs) given that a single TCR may recognize at least 106 different MHC-bound peptides (22, 23). Indeed, the epitope binds to the HLA molecule with specific residues in fixed positions along the sequence (anchor residues) and only the central residues are exposed for recognition by the TCR (http://www.cbs.dtu.dk/services/NetMHC/logos.php) (24, 25). Therefore, two unrelated antigens sharing the same TCR-facing central residues, or showing conservative variations at those positions, are very likely recognized by the same TCRs even if the peripheral residues are different, without affecting the structural conformation of the entire epitope.

Based on this assumption, the inventors have recently shown that tumor associated antigens (hereinafter referred as TAAs) described in the literature and publicly available at cancer peptide database (https://caped.icp.ucl.ac.be/Peptide/list) share sequence homology to viral sequences (26). Such homology suggest that viral antigens will elicit memory CD8⁺ T cells which may cross-react with tumor antigens controlling the growth of a cancer developed during the lifetime, if the expressed TAA is similar to the viral epitope. This may ultimately represent a relevant selective advantage for cancer patients and may lead to a novel preventive anti-cancer vaccine strategy (27).

WO2018/065628 provides a method to identify bacterial proteins in the human microbiome and to identify peptides from these bacterial proteins that can be presented by specific MHC molecules and specifically identifyies bacterial peptides IL13RA2 as candidate peptides for prevention or treatment of cancer.

Given the need to identify alternative cancer therapeutics and the limitations encountered in this field, such as the induced immune tolerance and/or undesired autoimmunity side effects, the present invention has the object of providing novel microbiota sequence variants of tumor associated antigenic epitope sequences and their use in cancer immunotherapies.

### SUMMARY OF THE INVENTION

The present invention relates to the identification of peptides derived from microbiota species of the Firmicutes and Bacteroidetes phyla sharing sequence homology to tumor-associated antigens (TAAs).

It is an object of the invention a composition comprising at least one peptide derived from the human microbiota and at least one pharmaceutically acceptable excipient, wherein said at least one peptide comprises a core sequence of 9 amino acids wherein said core sequence has 6, 7 or 8 amino acids identical and in the same position with respect to the tumor-associated antigenic epitope sequence GLYDGMEHL (SEQ ID No 1) and the remaining amino acids are different with respect to the amino acids in the same position of said epitope sequence.

Preferably said at least one peptide is selected from the group consisting of SEQ ID No. 13-90, more preferably from the group consisting of SEQ ID No 13, 16, 21, 23, 24, 27, 28, 29, 30, 33, 36, 38, 39, 40, 41, 43, 44, 45, 46, 55, 66, 67, 72, 73, 74, 75, 76, 77, 78, 79, 82, 82, 84, 85, 86, 87 and 88, even more preferably is selected from the group consisting of SEQ ID No 13, 23, 24, 75, 76 and 79.

It is a further object of the invention a composition consisting of at least one peptide derived from the human microbiota wherein said at least one peptide comprises a core sequence of 9 amino acids wherein said core sequence has 6, 7 or 8 amino acids identical and in the same position with respect to the tumor-associated antigenic epitope sequence GLYDGMEHL (SEQ ID No 1) and the remaining amino acids are different with respect to the amino acids in the same position of said epitope sequence; and at least one pharmaceutically acceptable excipient. Preferably said at least one peptide is selected from the group consisting of SEQ ID No. 13-90, more preferably from the group consisting of SEQ ID No 13, 16, 21, 23, 24, 27, 28, 29, 30, 33, 36, 38, 39, 40, 41, 43, 44, 45, 46, 55, 66, 67, 72, 73, 74, 75, 76, 77, 78, 79, 82, 82, 84, 85, 86, 87 and 88, even more preferably is selected from the group consisting of SEQ ID No 13, 23, 24, 75, 76 and 79. Preferably the composition comprising or consisting of at least one peptide as defined above comprises at least one further peptide selected wherein said at least one further peptide comprises a core sequence of 9 amino acids wherein said core sequence has at least 6 amino acids identical and in the same position with respect to one tumor-associated antigenic epitope sequence selected from the group consisting of:

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI.

Preferably the at least one further peptide is selected from the group consisting of SEQ ID No. 91-631, preferably is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, or 196, preferably is selected from the group consisting of SEQ ID No 99, 100, 102, 134, 137, 138, 160, 162, 164, 182, 183, 184.

It is a further object of the invention a composition comprising at least one peptide derived from the microbiota, wherein said at least one peptide comprises a core sequence of 9 amino acids wherein said core sequence has at least 6 acids identical and in the same position with respect to one tumor-associated antigenic epitope sequence selected from the group consisting of:

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI;

and a pharmaceutically acceptable vehicle or excipient.

In a preferred embodiment, the composition of the invention comprises at least one peptide derived from the microbiota wherein the least one peptide comprises a core sequence of 9 amino acids wherein said core sequence has 6, 7 or 8 amino acids identical and in the same position with respect to one tumor-associated antigenic epitope sequence selected from the group consisting of:

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI;

and remaining aminoacids are different with respect to the aminoacids in the same position of said tumor-associated antigenic epitope sequence.

Preferably, said at least one peptide is selected from the list consisting of of SEQ ID No. 91-209 or SEQ ID No. 211-327 or SEQ ID No. 329-507 or SEQ ID No. 509-631; more preferably is selected from the list consisting of: SEQ ID No 91-197; even more preferably the at least one peptide is selected from the group consisting of SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, or 196, preferably is selected from the group consisting of SEQ ID No 99, 100, 102, 134, 137, 138, 160, 162, 164, 182, 183, 184. It is a further object of the invention, the composition as defined above for use in the treatment and/or prevention of cancer, wherein the cancer is characterized by increased expression of at least one of the following antigens: GnTV, LRPAP1, MAGE-A10, MAGE-A3, MAGE-C1, MAGE-C2, SSX-2, KM-HN-1, KM-HN-2, MAGE-A2, SAGE.

Preferably the cancer is a solid tumor, preferably the solid tumor is selected from melanoma, lung cancer bladder cancers, esophageal and head and neck cancers, and sarcomas, colorectal cancer, gastric cancer, cutaneous squamous cell carcinoma, undifferentiated pleomorphic sarcoma/myxofibrosarcoma, non-small cell lung cancer, diffuse large B-cell lymphoma, hepatocellular carcinoma, breast cancer, pancreatic cancer, gynecology cancer.

Still preferably the composition of the invention is for use in combination with a further anticancer therapeutic agent.

It is a further object of the invention a vaccine or immunogenic composition comprising the composition of peptides as above defined and a pharmaceutically acceptable vehicle or excipient and preferably an adjuvant. Preferably, said vaccine or immunogenic composition is for use in the treatment and/or prevention of cancer, preferably for use in the treatment and/or prevention of cancer characterized by increased expression of at least one of the following antigens: GnTV, LRPAP1, MAGE-A10, MAGE-A3, MAGE-C1, MAGE-C2, SSX-2, KM-HN-1, KM-HN-2, MAGE-A2, SAGE. Even more preferably, said vaccine or immunogenic composition is for use in the treatment and/or prevention of solid tumors, and, in a further preferred embodiment, the solid tumor is selected from melanoma, lung cancer bladder cancers, esophageal and head and neck cancers, and sarcomas, colorectal cancer, gastric cancer, cutaneous squamous cell carcinoma, undifferentiated pleomorphic sarcoma/myxofibrosarcoma, non-small cell lung cancer, diffuse large B-cell lymphoma, hepatocellular carcinoma, breast cancer, pancreatic cancer, gynecology cancer.

It is a further object of the invention a dietary supplement or replacement comprising at least one peptide derived from the human microbiota, wherein said at least one peptide comprises a core sequence of 9 amino acids wherein said core sequence has 6, 7 or 8 amino acids identical and in the same position with respect to the tumor-associated antigenic epitope sequence GLYDGMEHL (SEQ ID No 1) and the remaining amino acids are different with respect to the amino acids in the same position of said epitope sequence.

Preferably said at least one peptide is selected from the group consisting of SEQ ID No. 13-90. More preferably said at least one peptide is selected from the group consisting of SEQ ID No 13, 16, 21, 23, 24, 27, 28, 29, 30, 33, 36, 38, 39, 40, 41, 43, 44, 45, 46, 55, 66, 67, 72, 73, 74, 75, 76, 77, 78, 79, 82, 82, 84, 85, 86, 87 and 88, preferably is selected from the group consisting of SEQ ID No 13, 23, 24, 75, 76 and 79.

In a preferred embodiment, the above dietary supplement or replacement comprises at least one further peptide derived from the human microbiota, wherein said at least one further peptide comprises a core sequence of 9 amino acids wherein said core sequence has at least 6 amino acids identical and in the same position with respect to one tumor-associated antigenic epitope sequence selected from the group consisting of:

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI.

Preferably the at least one further peptide is selected from the group consisting of SEQ ID No. 91-631. More preferably the at least one further peptide is selected from the group consisting of SEQ ID No 91-197, preferably SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, or 196, preferably is selected from the group consisting of SEQ ID No 99, 100, 102, 134, 137, 138, 160, 162, 164, 182, 183, 184. It is a further object of the invention a dietary supplement or replacement comprising at least one peptide derived from the microbiota, wherein said at least one peptide comprises a core sequence of 9 amino acids wherein said core sequence has at least 6 amino acids identical and in the same position with respect to one tumor-associated antigenic epitope sequence selected from the group consisting of:
KVAELVHFL (SEQ ID No. 2); or
FLWGPRALV (SEQ ID No 3); or

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI.

It is a further object of the invention a dietary supplement or replacement comprising at least one peptide derived from the microbiota, wherein the least one peptide comprises a core sequence of 9 amino acids wherein said core sequence has 6, 7 or 8 amino acids identical and in the same position with respect to one tumor-associated antigenic epitope sequence selected from the group consisting of:

(SEQ ID No. 2)
KVAELVHFL;
or
(SEQ ID No 3)
FLWGPRALV;
or
(SEQ ID No 4)
VLPDVFIRV;
or
(SEQ ID No 5)
KVLEYVIKV;
or
(SEQ ID No 6)
KVVEFLAML;
or
(SEQ ID No 7)
ALKDVEERV;
or
(SEQ ID No 8)
KASEKIFYV;
or
(SEQ ID No 9)
NYNNFYRFL;
or
(SEQ ID No 10)
EYLSLSDKI;
or
(SEQ ID No 11)
EYLQLVFGI;
or
(SEQ ID No 12)
LYATVIHDI;

and remaining aminoacids are different with respect to the aminoacids in the same position of said tumor-associated antigenic epitope sequence.

Preferably, said at least one peptide is selected from the group consisting of SEQ ID No. 91-209 or SEQ ID No. 211-327 or SEQ ID No. 329-507 or SEQ ID No. 509-631; more preferably is selected from the group consisting of: SEQ ID No 91-197, preferably said at least one peptide is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, or 196, preferably is selected from the group consisting of SEQ ID No 99, 100, 102, 134, 137, 138, 160, 162, 164, 182, 183, 184.

In a preferred embodiment of the invention the dietary supplement or replacement of the invention is for use in the prevention and/or in the treatment of cancer, wherein the cancer is characterized by increased expression of at least one of the following antigens: GnTV, LRPAP1, MAGE-A10, MAGE-A3, MAGE-C1, MAGE-C2, SSX-2, KM-HN-1, KM-HN-2, MAGE-A2, SAGE, preferably wherein the cancer is a solid tumor, preferably said solid tumor is selected from melanoma, lung cancer bladder cancers, esophageal and head and neck cancers, and sarcomas, colorectal cancer, gastric cancer, cutaneous squamous cell carcinoma, undifferentiated pleomorphic sarcoma/myxofibrosarcoma, non-small cell lung cancer, diffuse large B-cell lymphoma, 1. hepatocellular carcinoma, breast cancer, pancreatic cancer, gynecology cancer. It is a further object of the invention the non therapeutic use of the dietary supplement of the invention in the preparation of a nutraceutical composition.

The expression “peptide derived from the microbiota”, as used herein, refers to a peptide sequence identified in the microbiota and which can be obtained, isolated, extracted or purified from the microbiota or can be synthesized, produced, expressed with methods and techniques known in the art.

According to the present invention, the peptides derived from the microbiota as sequence analogs of the TAAs identified can be advantageously used in anti-tumor vaccine therapies. In short, the administration of a vaccine comprising one or more peptides according to the present invention would have the effect of stimulating the immune system against such peptides. Therefore, the invention preferably provides a vaccine or immunogenic composition comprising the composition as defined above and a pharmaceutically acceptable vehicle or excipient and preferably an adjuvant. Preferably said vaccine or immunogenic composition is for use in the treatment or prevention of cancer, preferably for use in the treatment or prevention of a cancer as above defined.

In a further embodiment, the present invention relates to an antibody conjugated with one or more peptides, and/or variants and/or salts and in which the antibody is capable of recognizing antigen presenting cells (APCs), preferably dendritic cells.

The antibody according to the present invention is able to recognize type C lectin receptors such as DC-SIGN (CD209) on dendritic cells. In this way the share of peptide captured by the dendritic cells is significantly increased and the peptide is presented more effectively to T cells. Advantageously, according to the present invention, the peptides derived from the microbiota as such, or peptides in optimized forms, such as for example fused to antibodies capable of recognizing antigen presenting cells (APC), such as dendritic cells, can be administered as a vaccine. Peptides can be administered in alternative forms, such as for example nucleic acids coding for said peptides, or vectors comprising said nucleic acids or directly to cells, for example APC, which express the peptides.

Furthermore, according to the present invention, the peptides derived from the microbiota can advantageously be used to stimulate T cells ex vivo and then re-infuse said T cells in the patient either after the administration of a further therapeutic agent or in the absence of any specific pre-treatment.

The invention further relates to an engineered cell comprising a recombinant protein, or a polynucleotide encoding a recombinant protein, preferably said recombinant protein being a recombinant receptor, more preferably a receptor expressed on the surface of the immune cell receptor preferably, wherein the recombinant receptor specifically binds to at least one peptide of the composition as described above, preferably wherein the recombinant receptor is a recombinant T cell receptor (TCR) or a chimeric antigen receptor (CAR). Therefore, the present invention also relates to a T lymphocyte receptor capable of binding one or more peptides as previously defined in which the receptor optionally comprises one or more co-stimulatory domains. The receptor is, for example, a chimeric receptor for the antigen (CAR).

In another embodiment, the present invention relates to a nucleotide sequence (DNA or RNA) which codes for a peptide as described above or for an antibody as described above.

In a further embodiment, the present invention relates to a nucleotide sequence that codes for T lymphocyte receptor capable of binding one or more peptides as previously defined as previously defined.

In a further embodiment, the present invention relates to an expression vector comprising a nucleotide sequence as previously defined.

It is specified that in the context of this discussion, the expression pharmaceutical composition or composition also refers indiscriminately to an immunogenic composition or to a vaccine composition.

The composition according to the present invention is for use in the treatment of cancer, preferably for use in the treatment or prevention of a cancer characterized by increased expression of at least one of the following antigens: GnTV, LRPAP1, MAGE-A10, MAGE-A3, MAGE-C1, MAGE-C2, SSX-2, KM-HN-1, KM-HN-2, MAGE-A2, SAGE.

As used herein the expression “cancer characterized by increased expression of at least one of the following antigens: GnTV, LRPAP1, MAGE-A10, MAGE-A3, MAGE-C1, MAGE-C2, SSX-2, KM-HN-1, KM-HN-2, MAGE-A2, SAGE” refers to solid tumors. As herein used, solid tumors comprise melanoma, lung cancer bladder cancers, esophageal and head and neck cancers, and sarcomas, colorectal cancer, gastric cancer, cutaneous squamous cell carcinoma, undifferentiated pleomorphic sarcoma/myxofibrosarcoma, non-small cell lung cancer and diffuse large B-cell lymphoma.

Preferably the composition of the invention can be administered in combination with further therapeutic regimen in a patient in need thereof. Still preferably, the composition of the invention is administered to a subject prior to, simultaneously or sequentially with other therapeutic regimens or co-agents useful for treating, and/or stabilizing cancer and/or preventing cancer relapsing (e.g. multiple drug regimens), in a therapeutically effective amount. The composition according to the present invention can be administered in the same or different composition(s) and by the same or different route(s) of administration as said co-agents.

Said other therapeutic regimens or co-agents may be selected from the group consisting of radiation therapy, chemotherapy, surgery, targeted therapy (including small molecules, peptides and monoclonal antibodies), and anti-angiogenic therapy. Anti-angiogenic therapy is defined herein as the administration of an agent that directly or indirectly targets tumor-associated vasculature. Preferred anti-cancer agents include a chemotherapeutic agent, a targeted drug and or an immunotherapeutic agent, such as an immune checkpoint modulator.

The composition of the invention can also be used as a dietary supplement or replacement, meaning for example a food product or drinking product comprising the composition as defined herein.

According to the invention, an expression vector that induces the expression of a peptide comprises a nucleic acid, DNA or RNA, coding for the peptide. The vector may be a RNA vector, a DNA vector, a viral vector or a bacterial vector.

According to the invention, “microbiota sequence analog” refer to a peptide sequence and the term “peptide” includes peptide variants retaining the ability to bind to MHC class I or MHC class II molecules, for example peptides having one or two amino acids substituted by amino acids belonging to the same group.

As further example microbiota sequence analog of TAAs of the invention have sequence homology of at least 80%, preferably at least 90%, with respect to the sequence of one or more peptides, comprising or consisting of an amino acid sequence chosen from the sequences from SEQ ID No. 1-12 and retain the ability to binding MHC class I.

Preferably, the composition of the invention comprises peptides with a length from 9 amino acids to 30 amino acids, preferably from 9 amino acids to 20 amino acids, more preferably of 9 amino acids.

In detail, the peptides of 9 amino acids are optimal for binding the MHC of class I, while to bind the MHC of class II the peptides must be of at least 15 amino acids, preferably 20-30 amino acids. Advantageously, when the peptide according to the present invention or the peptides according to the present invention are able to bind MHC of class II, the latter have a length of at least 15 amino acids, preferably from 20 amino acids to 30 amino acids.

As used herein MHC class II indicates HLA class II, preferably HLA-DR, more preferably HLA-DR1, HLA-DR3, HLA-DR4 and/or HLA-DR11.

As used herein, MHC of class I indicates HLA of class I, preferably HLA-A and/or HLA-B, more preferably HLA-A.

In detail HLA-A is chosen from the group consisting of HLA-A*02:01, HLA-A*01:01, HLA-A*26:01, HLA-A*03:01 and/or HLA-A*24:02, preferably HLA-A*02:01 and/or HLA-A*24:02.

The peptide according to the present invention, or the peptide variant as defined above, may comprise additional portions of amino acids at the N-terminus or C-terminus, which are not necessarily part of the peptide portion that serves as an epitope for the MHC molecules.

However, these additional parts can be important to provide an efficient introduction of the peptide according to the present invention into the cells.

In a further embodiment of the present invention, the peptides derived from the microbiota are bound to an antibody, or a functional part of the antibody itself, in particular peptides can be inserted into a sequence of an antibody, so as to be specifically carried by the antibody or, for example, can be fused to an antibody, or inserted into an antibody, which is specific for dendritic cells.

Furthermore, the peptides derived from the microbiota or variant according to the present invention can be further modified or modified to improve the stability and/or the binding with the MHC molecules, in order to obtain a stronger immune response. Methods for optimizing a peptide sequence are well known in the art and include, for example, the introduction of reverse peptide bonds or non-peptide bonds.

In a reverse peptide bond the amino acid residues are not joined by peptide bonds (—CO—NH—), but the peptide bond is reversed. Such retro-inverse peptidomimetic peptides can be made using methods known in the art, such as those described in Meziere et al (1997) J. Immunol. 159, 3230-3237, incorporated herein by reference. This approach involves the creation of pseudopeptides containing changes that involve the skeleton and not the orientation of the side chains. Meziere et al (1997) show that these pseudopeptides are useful for MHC binding and helper T cell responses.

Retro-inverse peptides, which contain NH—CO bonds instead of CO—NH peptide bonds, are much more resistant to proteolysis.

A non-peptide bond can be, for example, one of the following: —CH₂—NH, —CH₂S—, —CH₂CH₂—, —CH═CH—, —COCH₂—, —CH(OH) CH₂-and-CH₂SO—. U.S. Pat. No. 4,897,445 provides a method for the solid phase synthesis of non-peptide bonds (—CH₂—NH) in polypeptide chains involving the use of polypeptides synthesized by standard procedures and the non-peptide bond synthesized by the reaction of an amino-aldehyde and an amino acid in the presence of NaCNBH₃.

The peptides comprising the sequences described above can be synthesized with further chemical groups present at their amino and/or carboxyl end groups, to improve the stability, bioavailability and/or affinity of the peptides. For example, hydrophobic groups such as the carbobenzyloxy, dansyl or t-butyloxycarbonyl groups can be added to the amino terminals of the peptides. Similarly, an acetyl group or a 9-fluorenylmethoxycarbonyl group can be placed at the amino terminals of the peptides. Furthermore, the hydrophobic group, the t-butyloxycarbonyl or an amide group can be added to the carboxy-terminus of the peptides.

Furthermore, the peptides according to the present invention can be synthesized in such a way as to alter their steric configuration. For example, the D isomer of one or more of the amino acid residues of the peptide can be used, rather than the usual L isomer. Further, at least one of the amino acid residues of the peptides of the invention can be replaced by one of the well known amino acid residues, not present in nature. Alterations such as these can serve to increase the stability, bioavailability and/or binding action of the peptides of the invention.

Similarly, a peptide or a variant of the peptide according to the present invention can be chemically modified or modified by reacting specific amino acids before or after the synthesis of the peptide. Examples for such modifications are well known in the art and are summarized for example in R. Lundblad, Chemical Reagents for Protein Modification, 3rd ed. CRC Press, 2005, which is incorporated herein by reference.

Chemical modification of amino acids includes, but is not limited to: modification by acylation, amidation, pyridoxylation of lysine, reductive alkylation, trinitrobenzylation of amino groups with 2,4,6-trinitrobenzenesulfonic acid (TNBS), amide modification of carboxylic groups and sulfhydryl modification by oxidation with performic acid of cysteine to cysteic acid, formation of mercurial derivatives, formation of mixed disulfides with other thiol compounds, reaction with maleimide, carboxymethylation with iodoacetic acid or iodoacetamide and carbamylation with cyanate at alkaline pH. In this regard, the methodologies for the chemical modification of proteins are well known to one skilled in the art.

In short, the modification of arginyl residues in proteins is often based on the reaction of vicinal dicarbonyl compounds such as phenylglyoxal, 2,3-butanedione and 1,2-cyclohexanedione to form an adduct. Another example is the reaction of methylglyoxal with arginine residues. Cysteine can be modified without the concomitant modification of other nucleophilic sites such as lysine and histidine.

Consequently, a large number of cysteine modification reagents are available. The websites of companies such as Sigma-Aldrich (http://www.sigma-aldrich.com) provide information on specific reagents.

The selective reduction of disulfide bonds in proteins is also well known. Disulfide bonds can be formed and oxidized during the heat treatment of biopharmaceutical products. Woodward's K reagent can be used to modify specific glutamic acid residues. N-(3-(dimethylamino) propyl)-N′-ethylcarbodiimide can be used to form intra-molecular connections between a lysine residue and a glutamic acid residue. For example, diethyl pyrocarbonate is a reagent for the modification of histidyl residues in proteins. Histidine can also be modified using 4-hydroxy-2-nonenal. The reaction of lysine residues and other α-amino groups is, for example, useful in the binding of peptides to surfaces or in the cross-linking of proteins/peptides. Lysine is the attachment site of poly (ethylene) glycol and the major modification site in protein glycosylation. The methionine residues in proteins can be modified for example with iodoacetamide, bromoethylamine and chloramine T.

Tetranitromethane and N-acetylimidazole can be used for the modification of tyrosyl residues. Cross-linking through the formation of di-tyrosine can be accomplished with hydrogen peroxide/copper ions.

N-bromosuccinimide, 2-hydroxy-5-nitrobenzyl bromide or 3-bromo-3-methyl-2-(2-nitrophenylmercapto)-3H-indole (BPNS-skatole) have been used in some tryptophan modification studies.

Successful modification of therapeutic proteins and peptides with PEG is often associated with a prolongation of the circulatory half-life, while cross-linking of proteins with glutaraldehyde, polyethylene glycol diacrylate and formaldehyde is used for the preparation of hydrogels.

Chemical modification of allergens for immunotherapy is often achieved by carbamylation with potassium cyanate.

In general, peptides and variants (at least those containing peptide bonds between amino acid residues) can be synthesized by the Fmoc-polyamide mode of solid phase peptide synthesis, as disclosed by Lukas et al. (Synthesis of the solid phase peptide under continuous flow conditions Proc. Natl Acad Sci USA, May 1981; 78 (5): 2791-2795). Temporary protection of the N-amino group is provided by the 9-fluorenylmethyloxycarbonyl (Fmoc) group.

### DESCRIPTION OF FIGURES

FIG. 1. Affinity and stability of paired peptides. The affinity to HLA*02:01 (A) and HLA*24:02 (B) molecules were predicted by NetMHCstabpan for each TAA and paired bacterial peptides. The affinity to HLA molecules of all bacterial peptides derived from species of the Firmicutes and Bacterioidetes phyla are represented (C). The affinity values (Aff) are expressed in nanomolarity (nM).

FIG. 2. Comparison of amino acid residues between paired peptides. The type of amino acid substitution was evaluated at each peptide position comparing the paired TAA and homologous peptides derived from microbiota. The type of substitutions at each position is represented as percentage of the total number of sequences.

FIG. 3. Structural predicted conformation of MAGE-A10 and bacterial paired peptides. The conformation of the paired peptides bound to the HLA-A*02:01 molecule is shown. (A) Contact points on the surface of molecules are shown. Black areas=contact points with HLA molecule; Light grey areas=contact points with the TCR α and β chains. (B) Hydrogen bonds between the peptides and the TCR α and β chains are shown in the black dotted circle.

FIG. 4. Structural predicted conformation of MAGE-A3 KVAELVHFL and bacterial paired peptides. The conformation of the paired peptides bound to the HLA-A*02:01 molecule is shown. (A) Contact points on the surface of molecules are shown. Black areas=contact points with HLA molecule; Light grey areas=contact points with the TCR α and β chains. (B) Hydrogen bonds between the peptides and the TCR α and β chains are shown in the black dotted circle.

FIG. 5. Structural predicted conformation of MAGE-A3 FLWGPRALV and bacterial paired peptides. The conformation of the paired peptides bound to the HLA-A*02:01 molecule is shown. (A) Contact points on the surface of molecules are shown. Black areas=contact points with HLA molecule; Light grey areas=contact points with the TCR α and β chains. (B) Hydrogen bonds between the peptides and the TCR α and β chains are shown in the black dotted circle.

### MATERIALS AND METHODS

Peptides from the cancer antigenic peptide database (https://caped.icp.ucl.ac.be/Peptide/list) were selected if predicted as strong binders (SB) restricted to the most frequent MHC class I alleles (26). Subsequently they were subjected to global protein BLAST against the bacteria sequences of the Firmicutes and Bacteroidetes phyla within the GeneBank non-redundant protein database. The search returned a large number of HLA-A*0201 and HLA-A*2402 restricted bacterial sequences sharing homology with selected TAAs and the majority of such homology was found for the HLA-A*0201 (Tables 1 and 2). Speficically, the following TAAs were selected and are reported in the Tables 1 and 2: GnTv (PMID 8642259); LRPAP1 (PMID 30115740); MAGEA10 (PMID 10352307), MAGE A3 (PMID 28337438); MAGE A12m (PMID 7805731); MAGE C1 (PMID 21461886); MAGE C2 (PMID 14999777); SSX-2 (PMID 11823502); KM-HN-1 (PMID 15447989); MAGE A2 (PMID 10473111); SAGE (PMID 16061876).

TABLE 1

| TAA | | | FIRMICUTES | | | |
|---|---|---|---|---|---|---|
| | | Aff | | Aff | | |
| Name | PEPTIDE | (nM) | PEPTIDE | (nM) | specie | Seq ID |
| MAGE-A10 | GLYDGMEHL | 6.96 | GLYDGMEYI | 3.48 | DUF177 domain-containing | WP_012101781.1 |
| | (SEQ ID No 1) | | (SEQ ID No 13) | | protein [Clostridium kluyveri] | |
| | | | GLYDSMEYV | 2.89 | LysM peptidoglycan-binding | MBQ5676691.1 |
| | | | (SEQ ID No 14) | | domain-containing protein | |
| | | | GLYDGMLGV | 2.94 | Zn-dependent hydrolase | MBC8599015.1 |
| | | | (SEQ ID No 15) | | | |
| | | | YLYDDMEHL | 3.33 | LPXTG cell wall anchor domain- | MBQ7053643.1 |
| | | | (SEQ ID No 16) | | containing protein | |
| | | | | | [Oscillospiraceae bacterium] | |
| | | | ALYDGMEEV | 2.81 | TPA: phosphoglycolate | HBI63224.1 |
| | | | (SEQ ID No 17) | | phosphatase | |
| | | | GLYDGMNTV | 3.61 | response regulator | MBQ9361370.1 |
| | | | (SEQ ID No 18) | | | |
| | | | YLYNGIEHI (SEQ | 3.71 | MULTISPECIES: AAA family | WP_118553162.1 |
| | | | ID No 19) | | ATPase [Faecalibacterium] | |
| | | | VMYDGMEYL | 3.77 | DUF5598 domain-containing | MBQ2982712.1 |
| | | | (SEQ ID No 20) | | protein | |
| | | | GLYDGVEHV | 3.79 | S-layer homology domain- | WP_185142326.1 |
| | | | (SEQ ID No 21) | | containing protein [Cohnella | |
| | | | | | nanjingensis] | |
| | | | KLYDGLESL (SEQ | 4.76 | TPA: RNA polymerase sigma | HIU50197.1 |
| | | | ID No 22) | | factor RpoD [Candidatus | |
| | | | | | Limousia pullorum] | |
| | | | KLYDGIEHL (SEQ | 5.01 | MarR family winged helix-turn- | WP_117516162.1 |
| | | | ID No 23) | | helix transcriptional regulator | |
| | | | | | [[Absiella sp. AM29-15] | |
| | | | MLYDGMERL | 5.29 | fibronectin type III domain | EDS01593.1 |
| | | | (SEQ ID No 24) | | protein [[Eubacterium] siraeum | |
| | | | | | DSM 15702]] | |
| | | | MLYDGMERL 17 | 5.29 | fibronectin type III domain | EDS01593.1 |
| | | | (SEQ ID No 25) | | protein [[Eubacterium] siraeum | |
| | | | | | DSM 15702]] | |
| | | | SLYDGLEQL (SEQ | 5.58 | aminotransferase class I/II-fold | MBQ7065458.1 |
| | | | ID No 26) | | pyridoxal phosphate-dependent | |
| | | | | | enzyme | |
| | | | RLYDGLEHL (SEQ | 5.83 | sensor histidine kinase | WP_132012393.1 |
| | | | ID No 27) | | [Hydrogenispora ethanolica] | |
| | | | GLYEGMNHL | 7.01 | RsbU [[Staphylococcus aureus]] | CAC8447084.1 |
| | | | (SEQ ID No 28) | | | |
| | | | ALYDGMEQL | 7.16 | thiamine-phosphate kinase | WP_008590666.1 |
| | | | (SEQ ID No 29) | | [[Salimicrobium jeotgali]] | |
| | | | GLFDSMEHL | 8.99 | FprA family A-type flavoprotein | MBQ2769940.1 |
| | | | (SEQ ID No 30) | | [[Clostridia bacterium]] | |
| | | | GMYDMMEHL | 9.72 | diguanylate cyclase (GGDEF) | SCY45127.1 |
| | | | (SEQ ID No 31) | | domain-containing protein | |
| | | | GIYDGMEYI (SEQ | 9.74 | elongation factor 4 | GFI38123.1 |
| | | | ID No 32) | | | |
| | | | QLYNGMEHV | 9.89 | L, D-transpeptidase family | MBQ7360485.1 |
| | | | (SEQ ID No 33) | | protein [[Lachnospiraceae | |
| | | | | | bacterium]] | |
| | | | QLYNGMEHV | 9.89 | L, D-transpeptidase family | MBQ7360485.1 |
| | | | (SEQ ID No 34) | | protein | |
| | | | GLYEAMEEA | 10.19 | oxidoreductase, aldo/keto | EDO61928.1 |
| | | | (SEQ ID No 35) | | reductase family protein | |
| | | | | | [[Clostridium]] leptum DSM 753] | |
| | | | GLYDVMEHI | 10.66 | TPA: AAC(3) family N- | HHT96763.1 |
| | | | (SEQ ID No 36) | | acetyltransferase [Clostridiales | |
| | | | | | bacterium]] | |
| | | | GLFDGMENA | 11.23 | Ig-like domain-containing | MBO5371573.1 |
| | | | (SEQ ID No 37) | | protein | |
| | | | LLYAGMEHL | 13.06 | IS1182 family transposase | MBQ5522151.1 |
| | | | (SEQ ID No 38) | | [[Oscillospiraceae bacterium]] | |
| | | | KLYDGMEDL | 13.93 | HAD hydrolase-like protein | MBR0350109.1 |
| | | | (SEQ ID No 39) | | [Clostridia bacterium]] | |
| | | | GLYETMEHL | 14.31 | GNAT family N-acetyltransferase | MBQ3010667.1 |
| | | | (SEQ ID No 40) | | [Oscillospiraceae bacterium]] | |
| | | | ALYDTMEHL | 14.52 | CapA family protein | WP_173629024.1 |
| | | | (SEQ ID No 41) | | [Brevibacillus sp. MS2.2]] | |
| | | | GLYDGMNRA | 17.9 | glycosyltransferase | MBQ3790769.1 |
| | | | (SEQ ID No 42) | | | |
| | | | GLYSGMEHI | 19.91 | 1-deoxy-D-xylulose-5-phosphate | WP_206458099.1 |
| | | | (SEQ ID No 43) | | synthase [Anaerovorax sp. | |
| | | | | | IOR16]] | |
| | | | GLYDGMEFT | 20.69 | S-layer homology domain- | WP_063441043.1 |
| | | | (SEQ ID No 44) | | containing protein [Bacillus | |
| | | | | | badius]] | |
| | | | NLYDNMEHL | 21.02 | cobalt-precorrin-6A reductase | WP_095131333.1 |
| | | | (SEQ ID No 45) | | [Anaeromicrobium sediminis]] | |
| | | | GLYEGMKHL | 23.37 | ABC transporter permease | WP_098095089.1 |
| | | | (SEQ ID No 46) | | [[Bacillus wiedmannii]] | |
| | | | TLYDGMNDL | 26.72 | ABC transporter permease | MBS5732462.1 |
| | | | (SEQ ID No 47) | | [[Eubacterium]] siraeum]] | |
| | | | VLNDGMEHL | 27.42 | gliding motility protein | MBF1006940.1 |
| | | | (SEQ ID No 48) | | | |
| | | | GLYDGMVFS | 29.84 | methyltransferase domain- | MBQ1394714.1 |
| | | | (SEQ ID No 49) | | containing protein | |
| | | | GIYDGMETA | 54.65 | extracellular solute-binding | NBH26289.1 |
| | | | (SEQ ID No 50) | | protein | |
| | | | TLYDGMQQC | 60.42 | insulinase family protein | HIR09928.1 |
| | | | (SEQ ID No 51) | | [[Candidatus Avoscillospira | |
| | | | | | stercoripullorum]] | |
| | | | KLYDGMEVC | 66.68 | RNA-binding S4 domain- | MBQ7706286.1 |
| | | | (SEQ ID No 52) | | containing protein | |
| | | | NIYDGLEHL (SEQ | 67.82 | DUF262 domain-containing | MBS5512791.1 |
| | | | ID No 53) | | protein [[Faecalibacterium | |
| | | | | | prausnitzii]] | EOS74277.1 |
| | | | GVFEGMEHL | 72.18 | glycosyltransferase | |
| | | | (SEQ ID No 54) | | | |
| | | | GLYDGMEVT | 86.95 | HlyD family efflux transporter | WP_212975324.1 |
| | | | (SEQ ID No 55) | | periplasmic adaptor subunit | |
| | | | | | [[Bacillus sp. J14TS2]] | |
| | | | VIYDAMEHL | 136.6 | sensor histidine kinase KdpD | MBS6705371.1 |
| | | | (SEQ ID No 56) | | | |
| | | | VIYDAMEHL | 136.6 | DUF4118 domain-containing | MBS4967628.1 |
| | | | (SEQ ID No 57) | | protein | |
| | | | GIYDGMEKI (SEQ | 136.88 | transcription-repair coupling | MBQ6259525.1 |
| | | | ID No 58) | | factor, | |
| | | | HLTDGMEHL | 136.93 | energy-coupling factor | MBQ3104720.1 |
| | | | (SEQ ID No 59) | | transporter transmembrane | |
| | | | | | protein EcfT | |
| | | | PLYDGMETL | 194.53 | type II secretion system F | MBQ6814910.1 |
| | | | (SEQ ID No 60) | | family protein | |
| | | | SMKDGMEHL | 216.7 | ABC transporter substrate- | MBO5284181.1 |
| | | | (SEQ ID No 61) | | binding protei | |
| | | | TLYDGMRQT | 289.26 | TPA: DUF5110 domain- | HIS24277.1 |
| | | | (SEQ ID No 62) | | containing protein [[Candidatus | |
| | | | | | Faeciplasma gallinarum]] | |

| | | | BACTERIOIDES | | | |
|---|---|---|---|---|---|---|
| | | Aff | | Aff | | |
| Name | PEPTIDE | (nM) | PEPTIDE | (nM) | specie | Seq ID |
| MAGE-A10 | GLYDGMEHL | 6.96 | YLYDGLDHI | 2.41 | RHS repeat protein, partial | KAB6529547.1 |
| | (SEQ ID No 1) | | (SEQ ID No 63) | | | |
| | | | FLYDSVAHL | 3.11 | DNA repair protein RecO C- | UKI38930.1 |
| | | | (SEQ ID No 64) | | terminal domain-containing | |
| | | | | | protein [Alistipes putredinis] | |
| | | | VLYDGIEHV | 3.48 | HAD hydrolase-like protein | WP_134985449.1 |
| | | | (SEQ ID No 65) | | | |
| | | | GMYDGLEHL | 5.53 | LacI family transcriptional | RYZ62962.1 |
| | | | (SEQ ID No 66) | | regulator [[Chitinophagaceae | |
| | | | | | bacterium]] | |
| | | | GLYDGLENL | 6.22 | ORF6N domain-containing | WP_046368370.1 |
| | | | (SEQ ID No 67) | | protein [[Flavihumibacter | |
| | | | | | petaseus]] | |
| | | | TLADGMEYL | 6.78 | Methylmalonyl-CoA mutase large | KAA6301718.1 |
| | | | (SEQ ID No 68) | | subunit [[Candidatus Ordinivivax | |
| | | | | | streblomastigis] | |
| | | | YLYNGLKHL | 7.22 | TPA: pyruvate kinase [Candidatus | HIU39037.1 |
| | | | (SEQ ID No 69) | | Limisoma intestinavium] | |
| | | | RLYDGMDEL | 8.1 | HAD family hydrolase [Alistipes | WP_004328988.1 |
| | | | (SEQ ID No 70) | | putredinis]] | |
| | | | RLYDGMDAL | 8.45 | phosphoglycolate phosphatase | OKY94440.1 |
| | | | (SEQ ID No 71) | | [[Alistipes putredinis]] | |
| | | | GLYDGMELA | 9.65 | T9SS type A sorting domain- | MBN1398196.1 |
| | | | (SEQ ID No 72) | | containing protein [Bacteroidetes | |
| | | | | | bacterium]] | |
| | | | VLYDNMEHL | 12.56 | class I SAM-dependent | NCA71030.1 |
| | | | (SEQ ID No 73) | | methyltransferase | |
| | | | | | [Sphingobacteriia bacterium]] | |
| | | | GLYDEMEQL | 14.32 | HAD family hydrolase | WP_089782056.1 |
| | | | (SEQ ID No 74) | | [[Chitinophaga sp. YR573]] | |
| | | | GLYDGMEKA | 15.73 | SCO family protein | WP_135531157.1 |
| | | | (SEQ ID No 75) | | [Hymenobacter wooponensis]] | |
| | | | ALYDGMEDL | 17.29 | metallophosphoesterase | MBQ2520985.1 |
| | | | (SEQ ID No 76) | | [Paludibacteraceae bacterium]] | |
| | | | GLFDGMKHL | 18.76 | DUF2723 domain-containing | WP_194101443.1 |
| | | | (SEQ ID No 77) | | protein [[Mucilaginibacter sp. JRF] | |
| | | | ALYNGMDHL | 20.93 | T9SS type A sorting domain- | WP_071843189.1 |
| | | | | | containing protein [[Pontibacter | |
| | | | (SEQ ID No 78) | | korlensis]] | |
| | | | GLYDGMRHL | 21.51 | SulP family inorganic anion | WP_132555660.1 |
| | | | (SEQ ID No 79) | | transporter [[Niastella sp. CF465]] | |
| | | | GLYHGMNLL | 23.83 | restriction endonuclease subunit | WP_229034998.1 |
| | | | (SEQ ID No 80) | | S [[Alistipes putredinis]] | |
| | | | FVYDGTEHA | 27.69 | BspA family leucine-rich repeat | WP_188752688.1 |
| | | | (SEQ ID No 81) | | surface protein [[Parapedobacter | |
| | | | | | defluvii]] | |
| | | | ELYDGLEHL | 50.17 | formate acetyltransferase 2 | MBF8294546.1 |
| | | | (SEQ ID No 82) | | [[Bacteroidetes bacterium]] | |
| | | | YLYDGQEQF | 52.87 | MULTISPECIES: PD-(D/E)XK | WP_117678479.1 |
| | | | (SEQ ID No 83) | | nuclease family protein | |
| | | | | | [[Bacteroidales]] | |
| | | | AIYDGMEHA | 60.36 | UvrD-helicase domain-containing | WP_027076713.1 |
| | | | (SEQ ID No 84) | | protein [[Maribacter antarcticus]] | |
| | | | LLYDPKEHL | 73.05 | TPA: GNAT family N- | HIR83038.1 |
| | | | (SEQ ID No 85) | | acetyltransferase [[Candidatus | |
| | | | | | Cryptobacteroides pullicola]] | |
| | | | VTYDGMEHV | 78.18 | BspA family leucine-rich repeat | WP 188752688.1 |
| | | | (SEQ ID No 86) | | surface protein [[Parapedobacter | |
| | | | | | defluvii]] | |
| | | | FVYDGLEHT | 79.73 | BspA family leucine-rich repeat | WP 188752688.1 |
| | | | (SEQ ID No 87) | | surface protein [[Parapedobacter | |
| | | | | | defluvii]] | |
| | | | GLYDGMEKT | 82.74 | SCO family protein | MBC8082572.1 |
| | | | (SEQ ID No 88) | | [[Hymenobacter sp.]] | |
| | | | KTYDGLNHL | 157.46 | alpha/beta fold hydrolase | HIT15347.1 |
| | | | (SEQ ID No 89) | | [[Candidatus Avimuribaculum | |
| | | | | | pullicola]] | |
| | | | RTYEEMEQL | 641.66 | MULTISPECIES: conjugative | WP_044301905.1 |
| | | | (SEQ ID No 90) | | transposon protein TraN | |
| | | | | | [[Bacteroidales]] | |
| TAA | | | FIRMICUTES | | | |
| | | Aff | | Aff | | |
| Name | PEPTIDE | (nM) | PEPTIDE | (nM) | specie | Seq ID |
| MAGE-A3 | KVAELVHFL | 11.32 | YLTELLHFL (SEQ | 2.28 | Gfo/Idh/MocA family | MBE6834566.1 |
| | (SEQ ID No 2) | | ID No 91) | | oxidoreductase | |
| | | | KLAEMVHFL | 2.57 | glycosyltransferase family 2 | TGY95779.1 |
| | | | (SEQ ID No 92) | | protein | |
| | | | ILAELVNFV (SEQ | 2.98 | PA: recombinase family protein | HIV17749.1 |
| | | | ID No 93) | | | |
| | | | FVSELVHFI (SEQ | 4.79 | arginase | MBS6922347.1 |
| | | | ID No 94) | | | |
| | | | FQAELVNYL | 5.75 | TPA: phage tail sheath family | HIV18837.1 |
| | | | (SEQ ID No 95) | | protein | |
| | | | VLHELVHFL (SEQ | 5.84 | M48 family metallopeptidase | WP_097838615.1 |
| | | | ID No 96) | | [[Faecalibacterium prausnitzii]] | |
| | | | VLHELVHFL (SEQ | 5.84 | M48 family metallopeptidase | MBP3617361.1 |
| | | | ID No 97) | | | |
| | | | RLAELVDFL (SEQ | 5.92 | TPA: alpha-L-fucosidase | HIQ58336.1 |
| | | | ID No 98) | | | |
| | | | KIAELVHFL (SEQ | 7.1 | class I SAM-dependent | MBP8691684.1 |
| | | | ID No 99) | | methyltransferase | |
| | | | | | [[Sedimentibacter sp.]] | |
| | | | KIAEMVHFL | 7.41 | methyltransferase domain- | MBP7221824.1 |
| | | | (SEQ ID No 100) | | containing protein | |
| | | | | | [Sedimentibacter sp.] | |
| | | | KLAELVRFL (SEQ | 8.41 | diaminopimelate epimerase | MBR0093352.1 |
| | | | ID No 101) | | | |
| | | | AIAELVHFL (SEQ | 10.43 | phosphatidylglycerophosphatase | WP_054028593.1 |
| | | | ID No 102) | | A [[Bacillus sp. FJAT-28004]] | |
| | | | KVAELVLYL (SEQ | 10.7 | MATE family efflux transporter | MBQ6875242.1 |
| | | | ID No 103) | | | |
| | | | MVAELVQYL | 14.09 | TPA: DHH family | HIU31414.1 |
| | | | (SEQ ID No 104) | | phosphoesterase | |
| | | | RVAELIHFL (SEQ | 14.41 | dipeptidase PepV | MBQ3503822.1 |
| | | | ID No 105) | | [[Oscillospiraceae bacterium]] | |
| | | | KVAEFINFL (SEQ | 14.52 | GIY-YIG nuclease family protein | MBS7060157.1 |
| | | | ID No 106) | | [[Faecalibacterium prausnitzii]] | |
| | | | YAAELIHFL (SEQ | 14.67 | LacI family transcriptional | MBD5491386.1 |
| | | | ID No 107) | | regulator | |
| | | | KITELVHFL (SEQ | 15.99 | glycosyltransferase [[Clostridium | WP_035147891.1 |
| | | | ID No 108) | | tetanomorphum]] | |
| | | | HIAELVHFL (SEQ | 16.89 | phosphatidylglycerophosphatase | WP_143846559.1 |
| | | | ID No 109) | | A [[Bacillus sp. P16(2019)]] | |
| | | | KVSDLLHFL (SEQ | 21.03 | RNase adapter RapZ | MBQ9865489.1 |
| | | | ID No 110) | | | |
| | | | NVAEIVHFL (SEQ | 24.53 | precorrin-6A reductase | :MBC8577983.1 |
| | | | ID No 111) | | | |
| | | | TVADLVHYL | 24.73 | TPA: acyl carrier protein | HIT57844.1 |
| | | | (SEQ ID No 112) | | | |
| | | | SVAEAVHFL | 29.13 | TPA: bifunctional cobalt- | HBA64155.1 |
| | | | (SEQ ID No 113) | | precorrin-7 (C(5))- | |
| | | | | | methyltransferase/cobalt- | |
| | | | | | precorrin-6B (C(15))- | |
| | | | | | methyltransferase | |
| | | | KVAGLVLFL (SEQ | 30.9 | energy-coupling factor | HIV20050.1 |
| | | | ID No 114) | | transporter transmembrane | |
| | | | | | protein EcfT | |
| | | | QVAELVQFM | 33.27 | zinc ribbon domain-containing | MBR5337548.1 |
| | | | (SEQ ID No 115) | | protein | |
| | | | KVVETVHFL | 34.4 | radical SAM protein | MBT9700261.1 |
| | | | (SEQ ID No 116) | | | |
| | | | YAAELXHFL (SEQ | 36.76 | LacI family transcriptional | MBD5517959.1 |
| | | | ID No 117) | | regulator | |
| | | | MAAELVHFL | 48.55 | Glycosyltransferase involved in | SEA27257.1 |
| | | | (SEQ ID No 118) | | cell wall bisynthesis | |
| | | | RQSALVHFL | 54.27 | TPA: ATP-dependent RecD-like | HIU69400.1 |
| | | | (SEQ ID No 119) | | DNA helicase | |
| | | | MVAELVHIL | 56.03 | MATE family efflux transporter | MBR6359707.1 |
| | | | (SEQ ID No 120) | | | |
| | | | ATAEMLHFL | 57.39 | TPA: CpaF family protein | HIS65127.1 |
| | | | (SEQ ID No 121) | 63.77 | 3-oxoacyl-ACP reductase FabG | WP_188696003.1 |
| | | | QVAELVHFL | 76.88 | TPA: 50S ribosomal protein | HIR10503.1 |
| | | | (SEQ ID No 122) | | [[Pullulanibacillus camelliae]] | |
| | | | ELAELVHAL (SEQ | | L7/L12 | |
| | | | ID No 123) | | | |
| | | | SVAELVDFL (SEQ | 92.31 | TPA: AAA family ATPase | HBR05003.1 |
| | | | ID No 124) | | | |
| | | | AVTELVHRV | 112.64 | TPA: adenine | HIV10253.1 |
| | | | (SEQ ID No 125) | | phosphoribosyltransferase | |
| | | | KVTDLVQFL (SEQ | 136.1 | glycosyltransferase | MBP3701611.1 |
| | | | ID No 126) | | | |
| | | | IVAESVHFA (SEQ | 144.25 | single-stranded DNA-binding | RGT99395.1 |
| | | | ID No 127) | | protein | |
| | | | KVAELVNML | 150.7 | FAD-binding protein | MBC8578718.1 |
| | | | (SEQ ID No 128) | | | |
| | | | KVAELVDHL | 246.7 | LysM domain protein | EDO63073.1 |
| | | | (SEQ ID No 129) | | | |
| | | | KVAELVHVA | 451.92 | class II fructose-bisphosphate | MBR3171252.1 |
| | | | (SEQ ID No 130) | | aldolase | |
| | | | KVAELQHTA | 976.6 | TPA: flavin reductase family | HIU41270.1 |
| | | | (SEQ ID No 131) | | protein | |
| | | | KVAELQHTA | 976.6 | TPA: flavin reductase, partial | HIR41673.1 |
| | | | (SEQ ID No 132) | | | |
| TAA | | | BACTERIOIDES | | | |
| | | Aff | | Aff | | |
| Name | PEPTIDE | (nM) | PEPTIDE | (Nm) | specie | Seq ID |
| MAGE-A3 | KVAELVHFL | 11.32 | LLAENIHFL | 3.1 | thiamine-phosphate kinase | WP_004328936.1 |
| | (SEQ ID No 2) | | (SEQ ID No 133) | | [Alistipes putredinis] | |
| | | | VLAELVHFL | 3.31 | NADP-dependent | MGM3426064.1 |
| | | | (SEQ ID No 134) | | phosphogluconate | |
| | | | | | dehydrogenase [[Bacteroidetes | |
| | | | | | bacterium]] | |
| | | | KVAEFVALV | 4.84 | UvrD-helicase domain-containing | MBS6652293.1 |
| | | | (SEQ ID No 135) | | protein, partial [[Alistipes | |
| | | | | | putredinis]] | |
| | | | KVAEFIHFL | 5.34 | DEAD/DEAH box helicase family | MBS1647326.1 |
| | | | (SEQ ID No 136) | | protein [[Bacteroidetes | |
| | | | | | bacterium]] | |
| | | | KVAEFIHFL | 5.34 | GIY-YIG nuclease family protein | WP_207362944.1 |
| | | | (SEQ ID No 137) | | [[Fibrella sp. HMF5335]] | |
| | | | KVAELVHHV | 11 | response regulator [[Solirubrum | WP_078062927.1 |
| | | | (SEQ ID No 138) | | puertoriconensis]] | |
| | | | YIAELVEFA | 15.13 | replicative DNA helicase DnaB | BAG84094.1 |
| | | | (SEQ ID No 139) | | (plasmid) [[Candidatus | |
| | | | | | Azobacteroides | |
| | | | | | pseudotrichonymphae | |
| | | | | | genomovar. CFP2]] | |
| | | | KVADLEHFV | 16.05 | glutamine--fructose-6-phosphate | WP_004328005.1 |
| | | | (SEQ ID No 140) | | transaminase (isomerizing) | |
| | | | | | [[Alistipes putredinis]] | |
| | | | KVADLEHFV | 16.05 | glutamine--fructose-6-phosphate | MCD7972735.1 |
| | | | (SEQ ID No 141) | | transaminase (isomerizing) | |
| | | | | | [[Candidatus Azobacteroides sp.]] | |
| | | | KVTELVHFL | 30.35 | lantibiotic dehydratase | WP_169607473.1 |
| | | | (SEQ ID No 142) | | [[Mucilaginibacter sp. F39-2]] | |
| | | | LVAEILQFL | 31.53 | TPA: dihydropteroate synthase | HIU39050.1 |
| | | | (SEQ ID No 143) | | [[Candidatus Limisoma | |
| | | | | | intestinavium]] | |
| | | | KVAALVHFL | 31.63 | ATP-dependent DNA ligase | NBR83089.1 |
| | | | (SEQ ID No 144) | | [[Flavobacteriia bacterium]] | |
| | | | NVAELVPYL | 32.26 | TPA: DUF4493 domain-containing | HIU38292.1 |
| | | | (SEQ ID No 145) | | protein [[Candidatus Limisoma | |
| | | | | | intestinavium]] | |
| | | | KVAELVSFL | 34.3 | DEAD/DEAH box helicase family | WP_126321206.1 |
| | | | (SEQ ID No 146) | | protein [[Capnocytophaga | |
| | | | | | canimorsus]] | |
| | | | KVAELVNFL | 41.04 | DEAD/DEAH box helicase family | WP_095918246.1 |
| | | | (SEQ ID No 147) | | protein [[Capnocytophaga | |
| | | | | | canimorsus]] | |
| | | | QIAELTHFL | 43.36 | phosphatidylglycerophosphatase | KAB6698732.1 |
| | | | (SEQ ID No 148) | | A | |
| | | | SVAELIEFL | 44.33 | MULTISPECIES: phage integrase | WP_032598421.1 |
| | | | (SEQ ID No 149) | | SAM-like domain-containing | |
| | | | | | protein | |
| | | | QVAELIHFL | 48.21 | helix-turn-helix domain- | NQX86506.1 |
| | | | (SEQ ID No 150) | | containing protein | |
| | | | | | [[Flavobacteriaceae bacterium]] | |
| | | | GVASVIHFL | 48.34 | TPA: EamA family transporter | HIR37285.1 |
| | | | (SEQ ID No 151) | | [[Candidatus Limisoma | |
| | | | | | gallistercoris]] | |
| | | | ATAELVHFL | 62.15 | SpolIE family protein | MBQ3673518.1 |
| | | | (SEQ ID No 152) | | phosphatase [[Paludibacteraceae | |
| | | | | | bacterium]] | |
| | | | KVADLVHFA | 68.76 | glutamine--fructose-6-phosphate | NLC86325.1 |
| | | | (SEQ ID No 153) | | transaminase (isomerizing) | |
| | | | | | [[Bacteroidales bacterium]] | |
| | | | KVADLVHFA | 68.76 | TPA: glutamine--fructose-6- | HBT85187.1 |
| | | | (SEQ ID No 154) | | phosphate aminotransferase, | |
| | | | | | partial [[Porphyromonadaceae | |
| | | | | | bacterium]] | |
| | | | KVSELVNFL | 76.48 | lantibiotic dehydratase | MBC7417057.1 |
| | | | (SEQ ID No 155) | | [[Pedobacter sp.]] | |
| | | | KVSELVNFL | 76.48 | glycosyltransferase family 4 | WP_202104029.1 |
| | | | (SEQ ID No 156) | | protein [[Sphingobacterium sp. | |
| | | | | | C459-1T]] | |
| | | | SVSELINFL | 119.16 | site-specific integrase | KAB3841810.1 |
| | | | (SEQ ID No 157) | | | |
| | | | KIVELIKFL | 139.87 | integrase catalytic domain- | MBS6650716.1 |
| | | | (SEQ ID No 158) | | containing protein [[Alistipes | |
| | | | | | putredinis]] | |
| TAA | | | FIRMICUTES | | | |
| | | Aff | | Aff | | |
| Name | PEPTIDE | (nM) | PEPTIDE | (nM) | specie | Seq ID |
| MAGE-A12m | FLWGPRALV | 7,65 | FLWGPVMLV | 3.15 | sodium:alanine symporter family | QNM09157.1 |
| MAGE-A3 | (SEQ ID No 3) | | (SEQ ID No 159) | | protein | |
| | | | FLWAPIALV | 3.36 | D-alanyl-D-alanine | MBQ1820230.1 |
| | | | (SEQ ID No 160) | | carboxypeptidase [[Clostridia | |
| | | | | | bacterium]] | |
| | | | FLWGAPVLI (SEQ | 3.4 | sodium:alanine symporter family | HIR20419.1 |
| | | | ID No 161) | | protein [[Candidatus | |
| | | | | | Pelethomonas | |
| | | | | | intestinigallinarum]] | |
| | | | FLWGPFSLV | 3.71 | sodium:alanine symporter family | MBQ6708923.1 |
| | | | (SEQ ID No 162) | | protein [[Clostridia bacterium]] | |
| | | | YLWGLRALV | 4.72 | type II 3-dehydroquinate | NLY91350.1 |
| | | | (SEQ ID No 163) | | dehydratase [[Firmicutes | |
| | | | | | bacterium]] | |
| | | | FLWGGRALL | 5.24 | TPA: GNAT family N- | HHY09163.1 |
| | | | (SEQ ID No 164) | | acetyltransferase [[Firmicutes | |
| | | | | | bacterium]] | |
| | | | FLWDPRSGV | 6.17 | sulfatase [[Candidatus | HIR84741.1 |
| | | | (SEQ ID No 165) | | Galloscillospira | |
| | | | | | excrementavium]] | |
| | | | FLWGPFALI | 6.28 | sodium:alanine symporter family | MBC8587361.1 |
| | | | (SEQ ID No 166) | | protein [[Tissierellaceae | |
| | | | | | bacterium BX21]] | |
| | | | FLWGPAALI | 7.06 | sodium:alanine symporter family | WP_070611851.1 |
| | | | (SEQ ID No 167) | | protein [[Peptoniphilus sp. | |
| | | | | | HMSC062D09]] | |
| | | | YLWGKRALL | 9.77 | amino acid carrier protein | EHO34756.1 |
| | | | (SEQ ID No 168) | | | |
| | | | FLWNPRALL | 10.4 | O-antigen ligase family protein | MBQ6563626.1 |
| | | | (SEQ ID No 169) | | [[Clostridia bacterium]] | |
| | | | SLWGHRALV | 12.74 | cadmium-translocating P-type | MBR4261781.1 |
| | | | (SEQ ID No 170) | | ATPase [[Bacilli bacterium]] | |
| | | | TLWDSRALV | 13.15 | HAD family hydrolase | MBQ2288613.1 |
| | | | (SEQ ID No 171) | | | |
| | | | TLWDSRALV | 13.15 | HAD family hydrolase | MBQ2288162.1 |
| | | | (SEQ ID No 172) | | | |
| | | | FLWGPTVDA | 14.38 | TPA: DsrE family protein | HIT19511.1 |
| | | | (SEQ ID No 173) | | [[Candidatus Fimivivens | |
| | | | | | faecavium]] | |
| | | | FLWGSRGLV | 18.08 | HAMP domain-containing | WP_161846669.1 |
| | | | (SEQ ID No 174) | | histidine kinase | |
| | | | | | [[Pseudoflavonifractor sp. 524- | |
| | | | | | 17]] | |
| | | | FLWGKRAQA | 19.65 | alginate lyase family protein | |
| | | | (SEQ ID No 175) | | | |
| | | | FLWAPRAKI | 32.66 | cytochrome-c oxidase, cbb3- | |
| | | | (SEQ ID No 176) | | type subunit III [[Frateuria sp. | |
| | | | | | MAH-13]] | |
| | | | FLWGPRAKI | 39.11 | cytochrome-c oxidase, cbb3- | |
| | | | (SEQ ID No 177) | | type subunit III [[Bacillus sp. | |
| | | | | | SRB_336]] | |
| | | | FLWGPRQLA | 49.91 | MULTISPECIES: alpha/beta | |
| | | | (SEQ ID No 178) | | hydrolase [[unclassified | |
| | | | | | Lacticaseibacillus]] | |
| | | | FLHGPKALV | 49.92 | MFS transporter | MBO4679606.1 |
| | | | (SEQ ID No 179) | | | |
| | | | SLWGPRSAV | 65.81 | EAL domain-containing protein | QNM07838.1 |
| | | | (SEQ ID No 180) | | | |
| | | | ILFGPRALL (SEQ | 110.56 | TPA: RnfABCDGE type electron | HIR46659.1 |
| | | | ID No 181) | | transport complex subunit D | |
| | | | | | [[Candidatus Caccousia avicola]] | |
| TAA | | | BACTERIOIDES | | | |
| | | Aff | | Aff | | |
| Name | PEPTIDE | (nM) | PEPTIDE | (nM) | specie | Seq ID |
| MAGE-A12m | FLWGPRALV | 7,65 | FLWGSIALV | 2.41 | cation-translocating P-type | MBQ8420774.1 |
| MAGE-A3 | (SEQ ID No 3) | | (SEQ ID No 182) | | ATPase [[Bacteroidales bacterium]] | |
| | | | FLWESRALV | 3.02 | glycerol acyltransferase | MBF1567568.1 |
| | | | (SEQ ID No 183) | | [[Prevotella shahii]] | |
| | | | FLWGPHDLV | 4.66 | class I SAM-dependent | TAH08235.1 |
| | | | (SEQ ID No 184) | | methyltransferase | |
| | | | | | [[Sphingobacteriia bacterium]] | |
| | | | FLWNKRALV | 4.88 | response regulator [[Bacteroides | WP_204474554.1 |
| | | | (SEQ ID No 185) | | mediterraneensis]] | |
| | | | FLWNKRALV | 4.88 | TonB-dependent receptor | MBN2774199.1 |
| | | | (SEQ ID No 186) | | [[Prolixibacteraceae bacterium]] | |
| | | | FLWGPIGLV | 5.24 | A1-2E family transporter | MBE7199410.1 |
| | | | (SEQ ID No 187) | | [[Parafilimonas terrae]] | |
| | | | RLWGPIALV | 5.81 | FtsW/RodA/SpoVE family cell | WP_068823941.1 |
| | | | (SEQ ID No 188) | | cycle protein [[Wenyingzhuangia | |
| | | | | | fucanilytica]] | |
| | | | FMWGPRAEV | 6.08 | peptidylprolyl isomerase | MBL6865741.1 |
| | | | (SEQ ID No 189) | | [[Flavobacteriales bacterium]] | |
| | | | FLWGPKALV | 6.64 | fatty acid desaturase | MBL0883181.1 |
| | | | (SEQ ID No 190) | | [[Chitinophagaceae bacterium]] | |
| | | | FLWGLRALI | 8.29 | alanine:cation symporter family | WP_137344335.1 |
| | | | (SEQ ID No 191) | | protein [[Dyadobacter frigoris]] | |
| | | | FLYGVGALL | 8.9 | TPA: MFS transporter, partial | HIU38645.1 |
| | | | (SEQ ID No 192) | | [[Candidatus Limisoma | |
| | | | | | intestinavium]] | |
| | | | TLWGPAPLV | 12.91 | TPA: RagB/SusD family nutrient | HIQ85790.1 |
| | | | (SEQ ID No 193) | | uptake outer membrane protein | |
| | | | | | [[Candidatus Cryptobacteroides | |
| | | | | | excrementigallinarum]] | |
| | | | FLWGRRAVV | 14.24 | indolepyruvate ferredoxin | MBC8007764.1 |
| | | | (SEQ ID No 194) | | oxidoreductase family protein | |
| | | | | | [[Prolixibacteraceae bacterium]] | |
| | | | LLWGPKALV | 25.41 | fatty acid desaturase | NWK64964.1 |
| | | | (SEQ ID No 195) | | [[Sediminibacterium sp. Gen4]] | |
| | | | QLWGARALV | 34.92 | outer membrane protein | RQP14817.1 |
| | | | (SEQ ID No 196) | | assembly factor BamD, partial | |
| | | | | | [[Chryseobacterium sp.]] | |
| | | | AMWSPRAQV | 47.54 | TPA: tRNA uridine-5- | HIW87166.1 |
| | | | (SEQ ID No 197) | | carboxymethylaminomethyl(34) | |
| | | | | | synthesis enzyme MnmG, partial | |
| | | | | | [[Candidatus Onthomorpha | |
| | | | | | intestinigallinarum]] | |
| TAA | | | FIRMICUTES | | | |
| | | Aff | | Aff | | |
| Name | PEPTIDE | (nM) | PEPTIDE | (nM) | specie | Seq ID |
| GnTV | VLPDVFIRV | 7.59 | YLPDIFIRV (SEQ | 3.29 | HpcH/HpaI aldolase/citrate lyase | MBD5555280.1 |
| | (SEQ ID No 4) | | ID No 198) | | family protein [Roseburis sp.] | |
| | | | LLPDVLFRV (SEQ | 3.49 | polysaccharide biosynthesis | RGQ44771.1 |
| | | | ID No 199) | | protein [[Clostridium]] leptum]] | |
| | | | LLPDVLFRV (SEQ | 3.49 | polysaccharide biosynthesis | RGQ44771.1 |
| | | | ID No 200) | | protein [[Clostridium]] leptum] | |
| | | | FLQEIFIRV (SEQ | 3.99 | type IV secretory system | MBS4920904.1 |
| | | | ID No 201) | | conjugative DNA transfer family | |
| | | | | | protein [Faecalibacterium | |
| | | | | | prausnitzii]] | |
| | | | RLPDIFIRV (SEQ | 5.55 | HpcH/HpaI aldolase/citrate lyase | MBP1547557.1 |
| | | | ID No 202) | | family protein [Oscillospiraceae | |
| | | | | | bacterium]] | |
| | | | VLPDMFISV (SEQ | 5.88 | diguanylate cyclase | MBD5531855.1 |
| | | | ID No 203) | | | |
| | | | KLVDVLIRI | 6.78 | branched-chain amino acid ABC | EDS02017.1 |
| | | | (SEQ ID No 204) | | transporter, permease protein | |
| | | | | | [Eubacterium]] siraeum DSM | |
| | | | | | 15702]] | |
| | | | KLVDVLIRI (SEQ | 6.78 | branched-chain amino acid ABC | EDS02017.1 |
| | | | ID No 205) | | transporter, permease protein | |
| | | | | | [[Eubacterium] siraeum DSM | |
| | | | | | 15702]] | |
| | | | KLPDVFVSI (SEQ | 7.15 | cobalt ABC transporter | CDZ24285.1 |
| | | | ID No 206) | | permease [[Clostridium]] | |
| | | | | | cellulosi]] | |
| | | | KLPDVFVSI (SEQ | 7.15 | cobalt ABC transporter | CDZ24285.1 |
| | | | ID No 207) | | permease [[Clostridium]] | |
| | | | | | cellulosi]] | |
| | | | ALPEIFIRV (SEQ | 7.25 | TPA: ACT domain-containing | HIR19623.1 |
| | | | ID No 208) | | protein [Candidatus | |
| | | | | | Pelethomonas | |
| | | | | | intestinigallinarum]] | |
| | | | ALPEIFIRV (SEQ | 7.25 | TPA: ACT domain-containing | HIR19623.1 |
| | | | ID No 209) | | protein [Candidatus | |
| | | | | | Pelethomonas | |
| | | | | | intestinigallinarum]] | |
| | | | VLPDVFIRV (SEQ | 7.59 | ACT domain-containing protein | MBP0955392.1 |
| | | | ID No 210) | | [[Oscillospiraceae bacterium]] | |
| | | | VLPDIFIKV (SEQ | 7.67 | ACT domain-containing protein | NLO48847.1 |
| | | | ID No 211) | | [[Clostridiales bacterium]] | |
| | | | VLPDVFLRV | 7.77 | ACT domain-containing protein | MBQ2781239.1 |
| | | | (SEQ ID No 212) | | [[Clostridia bacterium]] | |
| | | | VLPEVFVRV | 7.91 | ACT domain-containing protein | WP_191543324.1 |
| | | | (SEQ ID No 213) | | [[Candidatus Avimonas caecicola]] | |
| | | | SLPEVFSRV (SEQ | 7.98 | ACT domain-containing protein | HIT18232.1 |
| | | | ID No 214) | | [[Candidatus Fimivivens | |
| | | | | | faecavium]] | |
| | | | SLPEVFSRV (SEQ | 7.98 | ACT domain-containing protein | HIT18232.1 |
| | | | ID No 215) | | [[Candidatus Fimivivens | |
| | | | | | faecavium]] | |
| | | | VLPEIFIKV (SEQ | 8.15 | ACT domain-containing protein | MBE6007693.1 |
| | | | ID No 216) | | | |
| | | | VLPEVFIKV (SEQ | 8.61 | ACT domain-containing protein | MBQ5332073.1 |
| | | | ID No 217) | | [[Oscillospiraceae bacterium]] | |
| | | | VLPEVFLKV (SEQ | 8.68 | oligosaccharide flippase family | WP_154251972.1 |
| | | | ID No 218) | | protein [[Faecalibacterium | |
| | | | | | prausnitzii]] | |
| | | | VLPEVFLKV (SEQ | 8.68 | amino acid-binding ACT protein | CDZ23284.1 |
| | | | ID No 219) | | [[Clostridium]] cellulosi]] | |
| | | | VLPEVFLKV (SEQ | 8.68 | amino acid-binding ACT protein | CDZ23284.1 |
| | | | ID No 220) | | [Clostridium]] cellulosi]] | |
| | | | VLPDVFEKV (SEQ | 12.88 | TPA: MFS transporter | HIR60123.1 |
| | | | ID No 221) | | [[Candidatus Faecivivens | |
| | | | | | stercoravium]] | |
| | | | VLPDVFEKV (SEQ | 12.88 | TPA: MFS transporter | HIR60123.1 |
| | | | ID No 222) | | [[Candidatus Faecivivens | |
| | | | | | stercoravium]] | |
| | | | ILPDVFIRL (SEQ | 13.37 | biotin synthase BioB | MBE5970738.1 |
| | | | ID No 223) | | [[Lachnospiraceae bacterium]] | |
| | | | ILPDVFIRL (SEQ | 13.37 | biotin synthase BioB | MBE5970738.1 |
| | | | ID No 224) | | | |
| | | | ELPDVFIRC (SEQ | 13.48 | extracellular solute-binding | WP_073279788.1 |
| | | | | | protein [[Anaerocolumna | |
| | | | ID No 225) | | jejuensis]] | |
| | | | GLPEVYIRV (SEQ | 13.84 | GNAT family N-acetyltransferase | MBD5533823.1 |
| | | | ID No 226) | | | |
| | | | VLDDVFIRI (SEQ | 16.57 | helix-turn-helix transcriptional | MBE5893125.1 |
| | | | ID No 227) | | regulator [ | |
| | | | ALTDVFIRI (SEQ | 18.69 | site-specific integrase | WP_055186996.1 |
| | | | ID No 228) | | [[Faecalibacterium prausnitzii]] | |
| | | | LLPDVFTRI (SEQ | 19.45 | amino acid carrier protein, | MBQ6833545.1 |
| | | | ID No 229) | | | |
| | | | ILPDVFRKV (SEQ | 25.79 | ACT domain-containing protein | HIS76208.1 |
| | | | ID No 230) | | [[Candidatus Merdivicinus | |
| | | | | | excrementipullorum]] | |
| | | | ILPDVFRKV (SEQ | 25.79 | TPA: ACT domain-containing | HIS76208.1 |
| | | | ID No 231) | | protein [[Candidatus | |
| | | | | | Merdivicinus | |
| | | | | | excrementipullorum]] | |
| | | | ILPDVFRKV (SEQ | 25.79 | ACT domain-containing protein | HIS76208.1 |
| | | | ID No 232) | | [[Candidatus Merdivicinus | |
| | | | | | excrementipullorum]] | |
| | | | ILPDVFRKV (SEQ | 25.79 | TPA: ACT domain-containing | HIS76208.1 |
| | | | ID No 233) | | protein [[Candidatus | |
| | | | | | Merdivicinus | |
| | | | | | excrementipullorum]] | |
| | | | ALPEVFRRV (SEQ | 31.24 | ACT domain protein PheB family | EDO61119.1 |
| | | | ID No 234) | | protein [[[[Clostridium]] leptum | |
| | | | | | DSM 753]] | |
| | | | ALPEVFRRV (SEQ | 31.24 | ACT domain protein PheB family | EDO61119.1 |
| | | | ID No 235) | | protein [[[[Clostridium]] leptum | |
| | | | | | DSM 753]] | |
| | | | VLPDTFIRL (SEQ | 37.23 | biotin synthase BioB | UEA73896.1 |
| | | | ID No 236) | | | |
| | | | VLPDVFIGA (SEQ | 39.15 | GerMN domain-containing | MBR2409146.1 |
| | | | ID No 237) | | protei | |
| | | | VLPEVFRKV (SEQ | 39.94 | ACT domain-containing protein | HIR41446.1 |
| | | | ID No 238) | | [[Candidatus Egerieicola | |
| | | | | | pullicola]] | |
| | | | VLPEVFRKV (SEQ | 39.94 | ACT domain-containing protein | HIR41446.1 |
| | | | ID No 239) | | [[Candidatus Egerieicola | |
| | | | | | pullicola]] | |
| | | | NLPDVFIRI (SEQ | 44.62 | MULTISPECIES: hypothetical | WP_002573979.1 |
| | | | ID No 240) | | protein [[Eubacteriales]] | |
| | | | QLPDVFVRI (SEQ | 61.93 | alanine:cation symporter family | MBS5309671.1 |
| | | | ID No 241) | | protein [[Faecalibacterium | |
| | | | | | prausnitzii]] | |
| | | | SLPDVFICA (SEQ | 64.67 | GGDEF domain-containing | MBR6147590.1 |
| | | | ID No 242) | | protein | |
| | | | FLPDVFIRC (SEQ | 99.54 | TPA: ATP-binding cassette | HEK51933.1 |
| | | | ID No 243) | | domain-containing protein, | |
| | | | | | partial [[Firmicutes bacterium]] | |
| | | | ELTDIFIRV (SEQ | 168.64 | TPA: oligosaccharide flippase | HIQ80887.1 |
| | | | ID No 244) | | family protein [[Candidatus | |
| | | | | | Scatavimonas merdigallinarum]] | |
| | | | ELTDIFIRV (SEQ | 168.64 | TPA: oligosaccharide flippase | HIQ80887.1 |
| | | | ID No 245) | | family protein [[Candidatus | |
| | | | | | Scatavimonas merdigallinarum]] | |
| TAA | | | BACTERIOIDES | | | |
| | | Aff | | Aff | | |
| Name | PEPTIDE | (nM) | PEPTIDE | (nM) | specie | Seq ID |
| GnTV | VLPDVFIRV | 7.59 | IMPDVFVPV | 4.53 | S41 family peptidase [Candidatus | HIR37852.1 |
| | (SEQ ID No 4) | | (SEQ ID No 246) | | Limisoma gallistercoris] | |
| | | | LLSDVFIRV | 4.93 | MULTISPECIES: | WP_118465647.1 |
| | | | (SEQ ID No 247) | | lipopolysaccharide biosynthesis | |
| | | | | | protein [[Bacteroides]] | |
| | | | FLPDIFIRI | 5.07 | dicarboxylate/amino acid:cation | MBR6655791.1 |
| | | | (SEQ ID No 248) | | symporter [[Alistipes sp.]] | |
| | | | FLPDVFIRI | 5.54 | dicarboxylate/amino acid:cation | MBR1962375.1 |
| | | | (SEQ ID No 249) | | symporter [[Alistipes sp.]] | |
| | | | ILQDVFIRV | 6.72 | RNA polymerase subunit sigma- | PGH37641.1 |
| | | | (SEQ ID No 250) | | 24 [[Candidatus Nephrothrix sp. | |
| | | | | | EaCA]] | |
| | | | ILQEVFIRV | 7.35 | RNA polymerase sigma factor | KAB3859120.1 |
| | | | (SEQ ID No 251) | | | |
| | | | ILPDVFVPI | 8.09 | TPA: S41 family peptidase | HIR83626.1 |
| | | | (SEQ ID No 252) | | [[Candidatus Cryptobacteroides | |
| | | | | | pullicola]] | |
| | | | GLPDVFLRV | 9.62 | FAD-binding oxidoreductase | MBM3443229.1 |
| | | | (SEQ ID No 253) | | [[Bacteroidetes bacterium]] | |
| | | | GLPDVFLRV | 9.62 | HAD-IIIA family hydrolase | WP_136842267.1 |
| | | | (SEQ ID No 254) | | [[Pedobacter sp. RP-3-22]] | |
| | | | GLPDVFLRV | 9.62 | HAD hydrolase family protein | WP_090992641.1 |
| | | | (SEQ ID No 255) | | [[Pedobacter insulae]] | |
| | | | VMPDAFIGV | 9.8 | tRNA (N(6)-L- | MCD7971285.1 |
| | | | (SEQ ID No 256) | | threonylcarbamoyladenosine(37)- | |
| | | | | | C(2))-methylthiotransferase MtaB | |
| | | | | | [[Candidatus Azobacteroides sp.]] | |
| | | | VLQEVFIRV | 10.08 | RNA polymerase sigma factor%2C | CUN36400.1 |
| | | | (SEQ ID No 257) | | sigma-70 family [[Bacteroides | |
| | | | | | finegoldii]] | |
| | | | VLPDTFIRV | 10.59 | sensory transduction protein LytT | GFD90141.1 |
| | | | (SEQ ID No 258) | | [[Tenacibaculum sp. KUL152]] | |
| | | | IMPDIFIPL | 10.6 | S41 family peptidase [[Candidatus | MCD7973287.1 |
| | | | (SEQ ID No 259) | | Azobacteroides sp.]] | |
| | | | IMPDVFVPL | 12.18 | TPA: S41 family peptidase | HIU38465.1 |
| | | | (SEQ ID No 260) | | [[Candidatus Limisoma | |
| | | | | | intestinavium]] | |
| | | | VLPDVFIGL | 17.68 | metallophosphoesterase | |
| | | | (SEQ ID No 261) | | [Bacteroidales bacterium] | MBO7397429.1 |
| | | | VLPDVFIAL | 19.98 | PD-(D/E)XK motif protein | RCU43886.1 |
| | | | (SEQ ID No 262) | | [[Chryseobacterium lacus]] | |
| | | | VLQDVFVKL | 24.12 | RNA polymerase sigma factor | KAA6301070.1 |
| | | | (SEQ ID No 263) | | YlaC [[Candidatus Ordinivivax | |
| | | | | | streblomastigis]] | |
| | | | IIPDAIIRV (SEQ | 32.64 | OmpA family protein [[Candidatus | MCD7972313.1 |
| | | | ID No 264) | | Azobacteroides sp.]] | |
| | | | ILPEVFARM | 41.77 | sodium-dependent transporter | MCD7972526.1 |
| | | | (SEQ ID No 265) | | [[Candidatus Azobacteroides sp.]] | |
| | | | VLPDVFVRA | 43.85 | PIG-L family deacetylase [[Alistipes | WP_204246478.1 |
| | | | (SEQ ID No 266) | | sp. An116]] | |
| | | | VLPDAFIRA | 90.14 | tetratricopeptide repeat protein | MBP7641748.1 |
| | | | (SEQ ID No 267) | | [[Saprospiraceae bacterium]] | |
| | | | VLPDIGIKI | 163.27 | preprotein translocase subunit | WP_055432820.1 |
| | | | (SEQ ID No 268) | | SecA [[Candidatus Symbiothrix | |
| | | | | | dinenymphae]] | |
| | | | KAPDVFIRV | 275.19 | glycosyltransferase family 4 | WP_229095086.1 |
| | | | (SEQ ID No 269) | | protein | |
| | | | VLHDVFVKT | 336.21 | TPA: sigma-70 family RNA | HIR36848.1 |
| | | | (SEQ ID No 270) | | polymerase sigma factor | |
| | | | | | [[Candidatus Limisoma | |
| | | | | | gallistercoris]] | |
| | | | ELPEVAIRV | 567.32 | TPA: tRNA pseudouridine(55) | HIU38848.1 |
| | | | (SEQ ID No 271) | | synthase TruB [[Candidatus | |
| | | | | | Limisoma intestinavium]] | |
| | | | VLRDIFINM | 637.19 | TPA: trimeric intracellular cation | HIT16500.1 |
| | | | (SEQ ID No 272) | | channel family protein | |
| | | | | | [[Candidatus Avimuribaculum | |
| | | | | | pullicola]] | |
| TAA | | | FIRMICUTES | | | |
| | | Aff | | Aff | | |
| Name | PEPTIDE | (nM) | PEPTIDE | (nM) | specie | Seq ID |
| LRPAP/ | KVLEYVIKV | 5.83 | RLLDYLIKV (SEQ | 2.56 | TPA: 30S ribosomal protein S15 | HIU39589.1 |
| MAGE-A1 | (SEQ ID No 5) | | ID No 273) | | [[Candidatus Limisoma | |
| | | | | | intestinavium]] | |
| | | | KVMEYLISI (SEQ | 2.99 | LysR family transcriptional | WP_158387495.1 |
| | | | ID No 274) | | regulator [[Faecalibacterium | |
| | | | | | prausnitzii]] | |
| | | | RLLEYIVKI (SEQ | 3.98 | MoxR family ATPase [[Candidatus | MCD7973973.1 |
| | | | ID No 275) | | Azobacteroides sp.′ | |
| | | | KVLEYVIRV (SEQ | 5.57 | CRISPR-associated helicase Cas3′ | NUJ26328.1 |
| | | | ID No 276) | | [[Firmicutes bacterium]] | |
| | | | KVLEYVAKV | 6.6 | formate C-acetyltransferase | WP_077853732.1 |
| | | | (SEQ ID No 277) | | [[Clostridium beijerinckii]] | |
| | | | KVLEYVIDV (SEQ | 7.39 | GAF domain-containing protein | WP_206808822.1 |
| | | | ID No 278) | | [[Desulfitobacterium sp. PLL0]] | |
| | | | KVLEYAIMV (SEQ | 7.57 | aspartyl/glutamyl- | SEP60213.1 |
| | | | ID No 279) | | tRNA(Asn/Gln) amidotransferase | |
| | | | | | subunit B | |
| | | | KVLEYVTKV | 7.83 | ATP-binding cassette domain- | WP_186300960.1 |
| | | | (SEQ ID No 280) | | containing protein | |
| | | | | | [[Staphylococcus pettenkoferi]] | |
| | | | KVLEYAIRV (SEQ | 10.34 | Asp-tRNA(Asn)/Glu-tRNA(Gln) | :MBE5895429.1 |
| | | | ID No 281) | | amidotransferase subunit GatB | |
| | | | KVLEYAIKV (SEQ | 11.22 | Asp-tRNA(Asn)/Glu-tRNA(Gln) | RKW64949.1 |
| | | | ID No 282) | | amidotransferase subunit GatB | |
| | | | | | [[Veillonella sp.]] | |
| | | | MVLDYMIKI | 12.84 | PF20097 family protein | WP_156076623.1 |
| | | | (SEQ ID No 283) | | [[Faecalibacterium prausnitzii]] | |
| | | | KVLEYIIKI (SEQ | 13.23 | ATP-binding protein | MBC7087093.1 |
| | | | ID No 284) | | [[Tissierellales bacterium]] | |
| | | | LVLEYLEKV (SEQ | 15.6 | flippase [[Faecalibacterium | WP 223384067.1 |
| | | | ID No 285) | | prausnitzii]] | |
| | | | TILEFAIKV (SEQ | 23.38 | TPA: RluA family pseudouridine | HIT15351.1 |
| | | | ID No 286) | | synthase [[Candidatus | |
| | | | | | Avimuribaculum pullicola]] | |
| | | | KTIEYVNKV | 24.62 | phospho-sugar mutase | HIT15315.1 |
| | | | (SEQ ID No 287) | | [[Candidatus Avimuribaculum | |
| | | | | | pullicola] | |
| | | | NVLGYVIKV (SEQ | 28.39 | RnfABCDGE type electron | MBR3806748.1 |
| | | | ID No 288) | | transport complex subunit G | |
| | | | KVLEYIIRA (SEQ | 34.23 | response regulator | MCD8014764.1 |
| | | | ID No 289) | | | |
| | | | SVLEYGIKV (SEQ | 43.69 | RNase adapter RapZ | MBO5354362.1 |
| | | | ID No 290) | | | |
| | | | KVLEYVDKI (SEQ | 86.52 | response regulator | MCC2168016.1 |
| | | | ID No 291) | | | |
| | | | KVLETVRKV | 212.37 | tetratricopeptide repeat protein | MCD7974005.1 |
| | | | (SEQ ID No 292) | | [[Candidatus Azobacteroides sp.]] | |
| | | | KVLEYIVKT (SEQ | 223.62 | FAD-dependent oxidoreductase | MBQ9562458.1 |
| | | | ID No 293) | | | |
| | | | KALEYVRNV | 232.38 | aspartate kinase [[Candidatus | HIR82602.1 |
| | | | (SEQ ID No 294) | | Cryptobacteroides pullicola]] | |
| | | | KVKEYVEKV (SEQ | 575.97 | DUF1351 domain-containing | WP_181573024.1 |
| | | | ID No 295) | | protein [[Faecalibacterium | |
| | | | | | prausnitzii]] | |
| | | | EILENVIKV (SEQ | 725.03 | glycosyltransferase family 4 | HIT15621.1 |
| | | | ID No 296) | | protein [[Candidatus | |
| | | | | | Avimuribaculum pullicola]] | |
| TAA | | | BACTERIOIDES | | | |
| | | Aff | | Aff | | |
| Name | PEPTIDE | (nM) | PEPTIDE | (nM) | specie | Seq ID |
| LRPAP/ | KVLEYVIKV | 5.83 | ALLEYVIKV | 3.04 | UxaA family hydrolase | WP_215235361.1 |
| MAGE-A1 | (SEQ ID No 5) | | (SEQ ID No 297) | | [[Dyadobacter sp. CECT 9623]] | |
| | | | HLLEYVIKV | 3.6 | altronate dehydratase family | WP_115829840.1 |
| | | | (SEQ ID No 298) | | protein [[Dyadobacter luteus]] | |
| | | | KVLEYLPKV | 5.07 | ABC transporter substrate- | HIT79747.1 |
| | | | (SEQ ID No 299) | | binding protein [[Candidatus | |
| | | | | | Faecivivens stercorigallinarum]] | |
| | | | RILEYVIKV (SEQ | 5.29 | altronate dehydratase family | WP_130140291.1 |
| | | | ID No 300) | | protein [[Sphingobacterium sp. | |
| | | | | | 30C10-4-7]] | |
| | | | KVLEYVAKV | 6.6 | RagB/SusD family nutrient uptake | MBK6621957.1 |
| | | | (SEQ ID No 301) | | outer membrane protein | |
| | | | | | [[Saprospirales bacterium]] | |
| | | | SMLEYVKKV | 8.29 | uroporphyrinogen decarboxylase | MBE6834075.1 |
| | | | (SEQ ID No 302) | | [[Clostridium]] sporosphaeroides]] | |
| | | | AVLEYVVKV | 8.61 | DUF2723 domain-containing | WP_173416227.1 |
| | | | (SEQ ID No 303) | | protein [[Mucilaginibacter mali]] | |
| | | | KVIEYLIEA | 10.65 | glutamate | WP_227088557.1 |
| | | | (SEQ ID No 304) | | formimidoyltransferase [[Alistipes | |
| | | | | | putredinis]] | |
| | | | KVLEYVEKV | 10.97 | LacI family transcriptional | WP_109650090.1 |
| | | | (SEQ ID No 305) | | regulator [[Maribacter | |
| | | | | | polysiphoniae]] | |
| | | | KVLEYCIKV | 13.52 | histidine--tRNA ligase family | HHE65310.1 |
| | | | (SEQ ID No 306) | | protein [[Bacteroidetes | |
| | | | | | bacterium]] | |
| | | | ALLEYLIKT (SEQ | 14.29 | MULTISPECIES: site-specific DNA- | WP_007569258.1 |
| | | | ID No 307) | | methyltransferase [[Bacteroidales]] | |
| | | | KVLEYVQGI | 14.92 | ABC transporter ATP-binding | MBS5732492.1 |
| | | | (SEQ ID No 308) | | protein [[Eubacterium]] siraeum]] | |
| | | | KVLNNVITV | 18.55 | flagellar biosynthesis protein FlhA | MBE6832653.1 |
| | | | (SEQ ID No 309) | | [[Clostridium]] sporosphaeroides]] | |
| | | | NVLEYVVKV | 20.59 | right-handed parallel beta-helix | MBP5995608.1 |
| | | | (SEQ ID No 310) | | repeat-containing protein | |
| | | | | | [[Crocinitomicaceae bacterium]] | |
| | | | MSLEYVLKV | 29.93 | CotH kinase family protein | WP_195610109.1 |
| | | | (SEQ ID No 311) | | | |
| | | | IVGEYVVKV | 30.03 | bifunctional chorismate | HIR84910.1 |
| | | | (SEQ ID No 312) | | mutase/prephenate dehydratase | |
| | | | | | [[Candidatus Galloscillospira | |
| | | | | | excrementavium]] | |
| | | | KVLEYIHKA | 31.49 | glycosyltransferase | WP_068855645.1 |
| | | | (SEQ ID No 313) | | | |
| | | | LVLEYVIKL | 33.91 | amidohydrolase [[Bacteroidia | MBK5280326.1 |
| | | | (SEQ ID No 314) | | bacterium] | |
| | | | KVLEYVKKV | 53.12 | argininosuccinate synthase | MBO75626541 |
| | | | (SEQ ID No 315) | | [Bacteroidales bacterium] | |
| | | | ATAEYVTKV | 58.93 | thymidine kinase [Allistipes | WP_215721925.1 |
| | | | (SEQ ID No 316) | | putredinis] | |
| | | | KVLEYNEEL | 69.07 | DEAD/DEAH box helicase family | WP_117829775.1 |
| | | | (SEQ ID No 317) | | protein | |
| | | | YFLEYVEKV | 125.24 | glycoside hydrolase family 1 | HIU506081 |
| | | | (SEQ ID No 318) | | protein [Candidatus Limousia | |
| | | | | | pollorum] | |
| | | | KYVLYVKRV | 531.53 | EAL domain-containing protein | HIT58468.1 |
| | | | (SEQ ID No 319) | | [Candidatus Faeciplasma | |
| | | | | | pullistercoris] | |
| TAA | | | FIRMICUTES | | | |
| | | Aff | | Aff | | |
| Name | PEPTIDE | (nM) | PEPTIDE | (nM) | specie | Seq ID |
| MAGE-C1 | KVVEFLAML | 25.53 | RLVEFLAML | 6.41 | Ger(x)C family spore | TLS53416.1 |
| MAGE-A3 | (SEQ ID No 6) | | (SEQ ID No 320) | | germination protein | |
| | | | | | [[Paenibacillus antri]] | |
| | | | KILEFLAML (SEQ | 8.49 | helix-turn-helix transcriptional | MBR5011159.1 |
| | | | ID No 321) | | regulator [[Clostridia bacterium]] | |
| | | | KLVAFLAML | 10.29 | beta-N-acetylglucosaminidase | MCC2147852.1 |
| | | | (SEQ ID No 322) | | domain-containing protein | |
| | | | KTVEFLAMV | 11.37 | GTPase | MBE5922580.1 |
| | | | (SEQ ID No 323) | | | |
| | | | ALFTFLAML (SEQ | 15.41 | TPA: AbgT family transporter | HIT18255.1 |
| | | | ID No 324) | | [[Candidatus Fimivivens | |
| | | | | | faecavium]] | |
| | | | KVADFLAML | 17.69 | phage tail tape measure protein | WP_105977819.1 |
| | | | (SEQ ID No 325) | | [[Staphylococcus simulans]] | |
| | | | KVIEFLSML (SEQ | 20.76 | helicase-exonuclease AddAB | HBP64347.1 |
| | | | ID No 326) | | subunit AddA | |
| | | | | | [[Desulfosporosinus sp.]] | |
| | | | KIVDFLAQA (SEQ | 22.32 | TPA: MerR family transcriptional | HIR03824.1 |
| | | | ID No 327) | | regulator [[Candidatus | |
| | | | | | Scatovicinus merdipullorum]] | |
| | | | KVVEFLAML | 25.53 | helix-turn-helix transcriptional | MBO5479965.1 |
| | | | (SEQ ID No 328) | | regulator [[Clostridia bacterium]] | |
| | | | GVIEFLAML (SEQ | 27.04 | ArsB/NhaD family transporter | WP_092482186.1 |
| | | | ID No 329) | | [[Desulfallas geothermicus]] | |
| | | | KIVEFISML | 32.53 | DNA helicase RecQ | NBI58092.1 |
| | | | (SEQ ID No 330) | | | |
| | | | AVVEILAML | 84.4 | sensor histidine kinase | WP_223385377.1 |
| | | | (SEQ ID No 331) | | [Faecalibacterium praunsnitzii] | |
| TAA | | | BACTERIOIDES | | | |
| | | Aff | | Aff | | |
| Name | PEPTIDE | (nM) | PEPTIDE | (nM) | specie | Seq ID |
| MAGE-C1 | KVVEFLAML | 25.53 | KVAEFVALV | 4.84 | UvrD-helicase domain-containing | WP_195272405.1 |
| MAGE-A3 | (SEQ ID No 6) | | (SEQ ID No 332) | | protein [[Alistipes putredinis]] | |
| | | | KVIEFLQDV | 8.68 | beta-N-acetylhexosaminidase | GAP71625.1 |
| | | | (SEQ ID No 333) | | [[Candidatus Symbiothrix | |
| | | | | | dinenymphae]] | |
| | | | KVIEFLALL | 9.11 | DUF2309 domain-containing | WP_111611765.1 |
| | | | (SEQ ID No 334) | | protein [[Algoriphagus yeomjeoni]] | |
| | | | CLNEFLPML | 9.39 | TPA: esterase [[Candidatus | HIR82174.1 |
| | | | (SEQ ID No 335) | | Cryptobacteroides pullicola]] | |
| | | | FVVDFLAQL | 10.12 | ATP-dependent helicase | WP_005802251.1 |
| | | | (SEQ ID No 336) | | | |
| | | | KVVDFLALL | 19.23 | type II secretion system protein | WP_210489586.1 |
| | | | (SEQ ID No 337) | | GspG [[Rufibacter sp. SYSU | |
| | | | | | D00433]] | |
| | | | RIVDFLALL | 19.25 | toprim domain-containing | WP_008640349.1 |
| | | | (SEQ ID No 338) | | protein | |
| | | | NLVEFIEML | 21.85 | carboxypeptidase-like regulatory | WP_117720041.1 |
| | | | (SEQ ID No 339) | | domain-containing protein | |
| | | | RIVDFLAQL | 22.59 | MULTISPECIES: toprim domain- | WP_004320436.1 |
| | | | (SEQ ID No 340) | | containing protein [[Bacteroidales]] | |
| | | | IVLEFLAML | 26.51 | nicotinamide mononucleotide | QCX40738.1 |
| | | | (SEQ ID No 341) | | transporter [[Flavobacteriaceae | |
| | | | | | bacterium 10Alg115]] | |
| | | | TVLEFLAML | 50.96 | CHAD domain-containing protein | MBL7846776.1 |
| | | | (SEQ ID No 342) | | [[Cyclobacteriaceae bacterium]] | |
| | | | SVAEFLEML | 63.35 | TPA: SUMF1/EgtB/PvdO family | HIT15828.1 |
| | | | (SEQ ID No 343) | | nonheme iron enzyme | |
| | | | | | [[Candidatus Avimuribaculum | |
| | | | | | pullicola]] | |
| | | | QVAEFLAML | 85.24 | heavy metal translocating P-type | QTN38443.1 |
| | | | (SEQ ID No 344) | | ATPase metal-binding domain- | |
| | | | | | containing protein | |
| | | | | | [[Cryomorphaceae bacterium]] | |
| | | | ELNEFLAML | 141.69 | TPA: TM2 domain-containing | HIQ84870.1 |
| | | | (SEQ ID No 345) | | protein [[Candidatus | |
| | | | | | Cryptobacteroides | |
| | | | | | excrementigallinarum]] | |
| TAA | | | FIRMICUTES | | | |
| | | Aff | | Aff | | |
| Name | PEPTIDE | (nM) | PEPTIDE | (nM) | specie | Seq ID |
| MAGE-C2 | ALKDVEERV | 239,61 | YLKEVEEPV (SEQ | 8.63 | TPA: transcriptional regulator | HIR60313.1 |
| | (SEQ ID No 7) | | ID No 346) | | | |
| | | | YLKDVEYRV (SEQ | 10.09 | CRISPR-associated protein Cas5, | EDS00205.1 |
| | | | ID No 347) | | Dvulg subtype | |
| | | | YLKDVEYRV (SEQ | 10.09 | type I-C CRISPR-associated | MBS5730812.1 |
| | | | ID No 348) | | protein Cas5 | |
| | | | LLSDVEERV (SEQ | 14.52 | TPA: rRNA pseudouridine | HIS24073.1 |
| | | | ID No 349) | | synthase | |
| | | | ALSDVEEGV | 17.22 | TPA: porphobilinogen synthase | :HIS65612.1 |
| | | | (SEQ ID No 350) | | | |
| | | | YLKDIDERV (SEQ | 20.38 | EAL domain-containing protein | MBE5851475.1 |
| | | | ID No 351) | | | |
| | | | YLKDIDERV (SEQ | 20.38 | GGDEF domain-containing | MBR3769223.1 |
| | | | ID No 352) | | protein, | |
| | | | ALSDVEERV | 23.82 | zinc-ribbon domain-containing | WP_125553189.1 |
| | | | (SEQ ID No 353) | | protein [[Loigolactobacillus | |
| | | | | | jiayinensis]] | |
| | | | ALNDVEERL | 51.02 | DNA repair protein RecN | NDO19858.1 |
| | | | (SEQ ID No 354) | | | |
| | | | MLKDIEERV | 51.51 | 50S ribosomal protein L11 | MBQ7934778.1 |
| | | | (SEQ ID No 355) | | methyltransferase [[Clostridia | |
| | | | | | bacterium]] | |
| | | | ALKDVEEPV | 52.68 | AMP-binding protein | MBQ7202456.1 |
| | | | (SEQ ID No 356) | | [[Eubacterium sp.]] | |
| | | | ALKDVEEPV | 52.68 | AMP-binding protein [[Clostridia | MBQ6558180.1 |
| | | | (SEQ ID No 357) | | bacterium]] | |
| | | | YLKNVEEMV | 56.46 | TPA: DUF4982 domain- | HIS24052.1 |
| | | | (SEQ ID No 358) | | containing protein | |
| | | | MLKEVEERV | 67.68 | MULTISPECIES: NAD(P)H- | WP_053430754.1 |
| | | | (SEQ ID No 359) | | dependent oxidoreductase | |
| | | | | | [[Alkalihalobacillus]] | |
| | | | ALTDVEEAL (SEQ | 68.45 | TPA: tRNA uridine-5- | HIR09109.1 |
| | | | ID No 360) | | carboxymethylaminomethyl(34) | |
| | | | | | synthesis GTPase MnmE | |
| | | | LLKDVEEQV | 93.41 | TPA: arginine--tRNA ligase | HBA96956.1 |
| | | | (SEQ ID No 361) | | | |
| | | | CLDDVEEKV | 110.75 | TPA: nucleotide sugar | HIT80107.1 |
| | | | (SEQ ID No 362) | | dehydrogenase | |
| | | | RLKEIEEQV (SEQ | 119.53 | TPA: B12-binding domain- | HIV18755.1 |
| | | | ID No 363) | | containing radical SAM protein | |
| | | | ALADIEERA | 164.41 | TPA: agmatinase | HIQ59651.1 |
| | | | (SEQ ID No 364) | | | |
| | | | GLKDIEEEL (SEQ | 182.42 | TPA: ABC transporter substrate- | HIQ57945.1 |
| | | | ID No 365) | | binding protein | |
| | | | ALKDVDAKV | 186.53 | 1,3-beta-galactosyl-N- | HIR10457.1 |
| | | | (SEQ ID No 366) | | acetylhexosamine | |
| | | | | | phosphorylase | |
| | | | TLKEIEEQV (SEQ | 200.53 | TPA: B12-binding domain- | HIQ59304.1 |
| | | | ID No 367) | | containing radical SAM protein | |
| | | | ELMDVEERV | 211.39 | sigma-70 family RNA polymerase | NSI91570.1 |
| | | | (SEQ ID No 368) | | sigma factor | |
| | | | NLQDVEERV | 231.29 | YggS family pyridoxal | MCD8338125.1 |
| | | | (SEQ ID No 369) | | phosphate-dependent enzym | |
| | | | ELMDVEERV | 231.39 | MULTISPECIES: sigma-70 family | WP_118486153.1 |
| | | | (SEQ ID No 370) | | RNA polymerase sigma factor | |
| | | | | | [[Eubacteriales]] | |
| | | | ALKDVEEAL (SEQ | 274.81 | iron-containing alcohol | MBQ7506690.1 |
| | | | ID No 371) | | dehydrogenase | |
| | | | FIKDVEERV (SEQ | 279.03 | serine hydrolase | MBD5494815.1 |
| | | | ID No 372) | | | |
| | | | ALNDVEKRV | 325.28 | ABC transporter substrate- | HIQ69970.1 |
| | | | (SEQ ID No 373) | | binding protein | |
| | | | ALRDLEEKV | 508.44 | AAA family ATPase | HIR83953.1 |
| | | | (SEQ ID No 374) | | | |
| | | | GLKDLEERL (SEQ | 661.49 | prokaryotic transcription | EDS00137.1 |
| | | | ID No 375) | | elongation factor, GreA/GreB | |
| | | | | | domain protein | |
| | | | ALKAVQERV | 790.13 | BREX system P-loop protein BrxC | MBR4606065.1 |
| | | | (SEQ ID No 376) | | | |
| | | | ALKSVEERL (SEQ | 1456. | glycosyltransferase family 36 | EEU95410.1 |
| | | | ID No 377) | 57 | [[Faecalibacterium prausnitzii | |
| | | | | | A2-165]] | |
| TAA | | | BACTERIOIDES | | | |
| | | Aff | | Aff | | |
| Name | PEPTIDE | (nM) | PEPTIDE | (nM) | specie | Seq ID |
| MAGE-C2 | ALKDVEERV | 239,61 | YLKDVEERV | 28.67 | K(+)-transporting ATPase subunit | WP_076782989.1 |
| | (SEQ ID No 7) | | (SEQ ID No 378) | | C [[Epilithonimonas bovis]] | |
| | | | FLKSYEEHL | 39.41 | site-specific integrase | WP_117928663.1 |
| | | | (SEQ ID No 379) | | | |
| | | | ALDDVEERV | 43.24 | peptidylprolyl isomerase | MBQ1773637.1 |
| | | | (SEQ ID No 380) | | [[Prevotella sp.]] | |
| | | | ALSNVVERL | 53.11 | TPA: outer membrane protein | HIT47270.1 |
| | | | (SEQ ID No 381) | | assembly factor BamA | |
| | | | | | [[Candidatus Cryptobacteroides | |
| | | | | | merdipullorum]] | |
| | | | SLKDVDESV | 74.1 | phosphoadenosine | WP_195272339.1 |
| | | | (SEQ ID No 382) | | phosphosulfate reductase family | |
| | | | | | protein [[Alistipes putredinis]] | |
| | | | ILKDIEERV | 99.47 | cation diffusion facilitator family | WP_188370161.1 |
| | | | (SEQ ID No 383) | | transporter [[Muriicola | |
| | | | | | marianensis]] | |
| | | | ALKEVAMRV | 116.23 | sensor protein KdpD [[Alistipes | MBS6650723.1 |
| | | | (SEQ ID No 384) | | putredinis]] | |
| | | | RLKELEEAV | 176.64 | site-specific integrase | WP_117928663.1 |
| | | | (SEQ ID No 385) | | | |
| | | | VINDVNSRV | 437.28 | site-specific integrase | WP_117928663.1 |
| | | | (SEQ ID No 386) | | | |
| | | | ALKSVEEKV | 921.33 | lactaldehyde dehydrogenase | RIB34267.1 |
| | | | (SEQ ID No 387) | | | |
| TAA | | | FIRMICUTES | | | |
| | | Aff | | Aff | | |
| Name | PEPTIDE | (nM) | PEPTIDE | (nM) | specie | Seq ID |
| SSX-2 | KASEKIFYV | 15,3 | RMIEKMFYV | 1.6 | minor capsid protein | HIV19461.1 |
| | (SEQ ID No 8) | | (SEQ ID No 388) | | [[Candidatus Merdivicinus | |
| | | | | | intestinigallinarum]] | |
| | | | FMREKIFYV (SEQ | 2.49 | N-6 DNA methylase | MBQ8814608.1 |
| | | | ID No 389) | | | |
| | | | KLSEKIFYL (SEQ | 2.59 | ATP-dependent DNA helicase | MBO4462507.1 |
| | | | ID No 390) | | | |
| | | | ALGEKIFYV (SEQ | 2.76 | sugar phosphate | MBP3875601.1 |
| | | | ID No 391) | | isomerase/epimerase | |
| | | | ALSEKIFYL (SEQ | 3.05 | J domain-containing protein | NBJ01656.1 |
| | | | ID No 392) | | | |
| | | | GLSEKIFYL (SEQ | 3.21 | ATP-dependent DNA helicase | MBR2530847.1 |
| | | | ID No 393) | | | |
| | | | KMSESVFTI (SEQ | 3.49 | HAMP domain-containing | HIR52329.1 |
| | | | ID No 394) | | histidine kinase [[Candidatus | |
| | | | | | Onthovicinus | |
| | | | | | excrementipullorum]] | |
| | | | GLTEKIFYL (SEQ | 3.66 | TPA: ATP-dependent DNA | HIT09333.1 |
| | | | ID No 395) | | helicase [[Candidatus | |
| | | | | | Merdivicinus faecavium]] | |
| | | | YASEKM FYV | 4.14 | SIS domain-containing protein | WP_193137167.1 |
| | | | (SEQ ID No 396) | | [[Lacticaseibacillus paracasei]] | |
| | | | SVNEKIFYV (SEQ | 5.39 | signal peptidase I | MBQ7724778.1 |
| | | | ID No 397) | | | |
| | | | VTAETIFYV (SEQ | 5.87 | MATE family efflux transporter | HIU41236.1 |
| | | | ID No 398) | | [[Candidatus Egerieicola faecale]] | |
| | | | GVSEKIFYV (SEQ | 6.18 | ferredoxin [[Clostridiales | OJU08501.1 |
| | | | ID No 399) | | bacterium 43-6]] | |
| | | | ANSEKIFYV (SEQ | 7.84 | TPA: 2-oxoacid:acceptor | HHY18381.1 |
| | | | ID No 400) | | oxidoreductase subunit alpha | |
| | | | | | [[Firmicutes bacterium]] | |
| | | | QLKEKLFYV (SEQ | 8.93 | Aspartyl aminopeptidase | CBL34879.1 |
| | | | ID No 401) | | [[Eubacterium]] siraeum | |
| | | | | | V10Sc8a]] | |
| | | | QLKEKLFYV (SEQ | 8.93 | Probable M18 family | CUQ83949.1 |
| | | | ID No 402) | | aminopeptidase 1 | |
| | | | | | [[Eubacterium]] siraeum]] | |
| | | | KAADKIFYV | 9.18 | A1-2E family transporter | MBQ6843512.1 |
| | | | (SEQ ID No 403) | | [[Agathobacter sp.]] | |
| | | | DATEKIFYV (SEQ | 9.55 | InlB B-repeat-containing protein | WP_039861470.1 |
| | | | ID No 404) | | [[Peptoniphilus lacrimalis]] | |
| | | | ELSDKIFYV (SEQ | 9.59 | leucine-rich repeat domain- | MBE5889045.1 |
| | | | ID No 405) | | containing protein | |
| | | | KAAEKFFYV | 10.1 | asparagine synthase (glutamine- | MBR2037171.1 |
| | | | (SEQ ID No 406) | | hydrolyzing) [[Lachnospiraceae | |
| | | | | | bacterium]] | |
| | | | KAAEKFFYV (SEQ | 10.1 | asparagine synthase | MBR2037171.1 |
| | | | ID No 407) | | | |
| | | | STLEKIIYV (SEQ | 11.81 | TPA: bis(5′-nucleosyl)- | HIR10297.1 |
| | | | ID No 408) | | tetraphosphatase (symmetrical) | |
| | | | | | Yqek, partial [[Candidatus | |
| | | | | | Avoscillospira stercoripullorum]] | |
| | | | KASDKIFYV (SEQ | 16.93 | A1-2E family transporter | MBQ8518875.1 |
| | | | ID No 409) | | [[Agathobacter sp.]] | |
| | | | KTAEKIFRV (SEQ | 20.34 | cysteine desulfurase family | EEG32253.1 |
| | | | ID No 410) | | protein [[[[Clostridium]] | |
| | | | | | methylpentosum DSM 5476]] | |
| | | | TTLEKIIYV (SEQ | 22.74 | nicotinate (nicotinamide) | HIR50649.1 |
| | | | ID No 411) | | nucleotide adenylyltransferase | |
| | | | | | [[Candidatus Avoscillospira | |
| | | | | | avicola]] | |
| | | | VQSEKIFPI (SEQ | 25.31 | ATPase/histidine kinase/DNA | EDO62734.1 |
| | | | ID No 412) | | gyrase B/HSP90 domain protein | |
| | | | | | [[Clostridium]] leptum DSM 753]] | |
| | | | VQSEKIFPI (SEQ | 25.31 | molecular chaperone HtpG | RGQ44838.1 |
| | | | ID No 413) | | [[[[Clostridium]] leptum]] | |
| | | | IAGEKIFYV (SEQ | 26.23 | MULTISPECIES: HAMP domain- | WP_057897585.1 |
| | | | ID No 414) | | containing histidine kinase | |
| | | | | | [[Aneurinibacillus]] | |
| | | | KANEKIYYV | 28.53 | isoleucine--tRNA ligase | WP_084053470.1 |
| | | | (SEQ ID No 415) | | [[Desulfonispora | |
| | | | | | thiosulfatigenes]] | |
| | | | NAAERIFYV (SEQ | 30.07 | glycosyltransferase family 2 | MCC2230127.1 |
| | | | ID No 416) | | protein | |
| | | | SASEKIYYV (SEQ | 37.86 | SulP family inorganic anion | WP_091273761.1 |
| | | | ID No 417) | | transporter [[Alteribacillus | |
| | | | | | persepolensis]] | |
| | | | KASEAIFYL (SEQ | 40.14 | TPA: DUF975 family protein | HIT94498.1 |
| | | | ID No 418) | | [[Candidatus Faecivivens | |
| | | | | | stercoripullorum]] | |
| | | | GSSEKIFYV (SEQ | 59.42 | TnsD family Tn7-like | WP_098398638.1 |
| | | | ID No 419) | | transposition protein [[Bacillus | |
| | | | | | cereus]] | |
| | | | TTAQKIFYV (SEQ | 59.63 | glycosyltransferase family 2 | HIR49715.1 |
| | | | ID No 420) | | protein [[Candidatus | |
| | | | | | Avoscillospira avicola]] | |
| | | | KAGDRIYYV (SEQ | 66.14 | aminopeptidase [[Candidatus | HIU49597.1 |
| | | | ID No 421) | | Limousia pullorum]] | |
| | | | GQSEKIFSA (SEQ | 119.79 | trypsin-like peptidase domain- | HIS76607.1 |
| | | | ID No 422) | | containing protein [[Candidatus | |
| | | | | | Merdivicinus | |
| | | | | | excrementipullorum]] | |
| | | | KTKEKVFYI (SEQ | 623.57 | peptidoglycan DD- | WP_005923638.1 |
| | | | ID No 423) | | metalloendopeptidase family | |
| | | | | | protein [[Faecalibacterium | |
| | | | | | prausnitzii]] | |
| TAA | | | BACTERIOIDES | | | |
| | | Aff | | Aff | | |
| Name | PEPTIDE | (nM) | PEPTIDE | (nM) | specie | Seq ID |
| SSX-2 | KASEKIFYV | 15,3 | YTSEKIFYV | 2.68 | SAM-dependent DNA | RYE15078.1 |
| | (SEQ ID No 8) | | (SEQ ID No 424) | | methyltransferase, partial | |
| | | | | | [[Sphingobacteriaceae bacterium]] | |
| | | | KLSEKNFYV | 2.75 | plasmid recombination protein | WP_117830273.1 |
| | | | (SEQ ID No 425) | | | |
| | | | KISEDVFYV | 3.01 | DNA replication/repair protein | MCD7971606.1 |
| | | | (SEQ ID No 426) | | RecF [[Candidatus Azobacteroides | |
| | | | | | sp.]] | |
| | | | VVSEDIFYV | 4.52 | FprA family A-type flavoprotein | MCD7972676.1 |
| | | | (SEQ ID No 427) | | [[Candidatus Azobacteroides sp.]] | |
| | | | KAYEKIFYV | 4.93 | alpha/beta hydrolase | MBQ6038980.1 |
| | | | (SEQ ID No 428) | | [[Bacteroidaceae bacterium]] | |
| | | | KVTEKIFYV | 5.36 | toxin-antitoxin system YwqK | MBK9284067.1 |
| | | | (SEQ ID No 429) | | family antitoxin | |
| | | | | | [[Sphingobacteriaceae bacterium]] | |
| | | | KTSEKVFYV | 9.03 | LPS-assembly protein LptD | HCQ15094.1 |
| | | | (SEQ ID No 430) | | [[Cryomorphaceae bacterium]] | |
| | | | KAAEKVFYV | 10.07 | GH92 family glycosyl hydrolase | MBQ9583024.1 |
| | | | (SEQ ID No 431) | | [[Bacteroidales bacterium]] | |
| | | | KTTEKIFYV | 10.46 | acyl-CoA desaturase [[Cytophagia | NBP69626.1 |
| | | | (SEQ ID No 432) | | bacterium]] | |
| | | | KVSEKIFYL | 10.49 | glycosyltransferase family 4 | WP_196828382.1 |
| | | | (SEQ ID No 433) | | protein [[Flavobacterium sp. | |
| | | | | | CG_9.1]] | |
| | | | GASERIFYV | 37.69 | aminodeoxychorismate lyase | MBP1646831.1 |
| | | | (SEQ ID No 434) | | [[Bacteroidetes bacterium]] | |
| | | | VASEKIFFV | 46.62 | UDP-N-acetylglucosamine 2- | WP_132989163.1 |
| | | | (SEQ ID No 435) | | epimerase (non-hydrolyzing) | |
| | | | | | [[Flavobacterium zhairuonensis]] | |
| | | | KISEKIRYV | 72.59 | FprA family A-type flavoprotein | HIS22046.1 |
| | | | (SEQ ID No 436) | | [[Candidatus Cryptobacteroides | |
| | | | | | intestinpullorum] | |
| | | | RASEKLYFV | 75.44 | ATP-binding domain-containing | UKI40112.1 |
| | | | (SEQ ID No 437) | | protein [Alisitipes putredinis] | |
| | | | VASEKTFYV | 75.82 | cytochrome c oxidase accessory | WP_027124754.1 |
| | | | (SEQ ID No 438) | | protein CcoG [Gelidibacter | |
| | | | | | mesophilus] | |

TABLE 2
Homology between HLA-A*24:02 limked TAA and microbiota species

| TAA | | | FIRMICUTES | | | |
|---|---|---|---|---|---|---|
| Name | PEPTIDE | Aff (nM) | PEPTIDE | Aff (nM) | specie | Seq ID |
| KM-HN-1 | NYNNFYRFL | 157.45 | NYTNFCRFF | 34.47 | phosphotransferase system. EIIC | EEU98063.1 |
| | (SEQ ID No 9) | | (SEQ ID No 439) | | [Faecalibacterium prausnitzii A2- | |
| | | | | | 165] | |
| | | | GYYNFYRNF | 41.77 | Lantibiotic modifying enzyme | CUQ81199.1 |
| | | | (SEQ ID No 440) | | | |
| | | | RYNGFYRFL | 44.03 | sugar ABC transporter permease | WP_155705275.1 |
| | | | (SEQ ID No 441) | | Paenibacillus psychroresistens | |
| | | | NYIRFYRFL | 50.33 | spore germination protein | MBO4988623.1 |
| | | | (SEQ ID No 442) | | [Lachnospiraceae bacterium] | |
| | | | NYNNFLQKI | 60.81 | LysM peptidoglycan-binding | PZM89890.1 |
| | | | (SEQ ID No 443) | | domain-containing protein | |
| | | | KYNNYYRFL | 65.88 | DEAD/DEAH box helicase | WP_154750440.1 |
| | | | (SEQ ID No 444) | | Planomicrobium sp. YIM 101495 | |
| | | | NYNSFYSFL | 75.01 | SufD family Fe-S cluster assembly | WP_191684926.1 |
| | | | (SEQ ID No 445) | | protein Limosilactobacillus sp. | |
| | | | | | Sa3CUN2 | |
| | | | NYNNFYVFL | 88.64 | glycosyltransferase family 2 protein | WP_088223547.1 |
| | | | (SEQ ID No 446) | | Anoxybacillus flavithermus | |
| | | | KYNEFYRLV | 90.51 | restriction endonuclease | WP_097770998.1 |
| | | | (SEQ ID No 447) | | [Faecalibacterium prausnitzii] | |
| | | | EYSNFYRAF | 106.98 | TPA: helix-turn-helix domain- | HIR10485.1 |
| | | | (SEQ ID No 448) | | containing protein | |
| | | | KYNDFYRYL | 110.61 | GNAT family N-acetyltransferase | MBS7105243.1 |
| | | | (SEQ ID No 449) | | [Faecalibacterium prausnitzii] | |
| | | | DYANFYRAF | 113.9 | AraC family transcriptional | RGC41348.1 |
| | | | (SEQ ID No 450) | | regulator [Faecalibacterium | |
| | | | | | prausnitzii] | |
| | | | DYANFYRAF | 113.9 | TPA: helix-turn-helix domain- | HIT09977.1 |
| | | | (SEQ ID No 451) | | containing protein | |
| | | | FYNNFYRNL | 121.17 | GntR family transcriptional | MBS7060054.1 |
| | | | (SEQ ID No 452) | | regulator [Faecalibacterium | |
| | | | | | prausnitzii] | |
| | | | SYGNFYRFM | 129.9 | FtsW/RodA/SpoVE family cell cycle | MBE5948340.1 |
| | | | (SEQ ID No 453) | | protein [Lachnospiraceae | |
| | | | | | bacterium] | |
| | | | SYNNLYRYL | 131.59 | DDE-type | MBR1633626.1 |
| | | | (SEQ ID No 454) | | integrase/transposase/recombinase | |
| | | | | | [Lachnospiraceae bacterium] | |
| | | | HYTDIYRFL | 142.97 | TPA: RNA polymerase sigma factor | HIT54277.1 |
| | | | (SEQ ID No 455) | | | |
| | | | DYNAFYRSF | 160.2 | AraC family transcriptional | WP_154265874.1 |
| | | | (SEQ ID No 456) | | regulator [Faecalibacterium | |
| | | | | | prausnitzii] | |
| | | | DYSNFYRAF | 207.62 | AraC family transcriptional | WP_143406190.1 |
| | | | (SEQ ID No 457) | | regulator [Faecalibacterium | |
| | | | | | prausnitzii] | |
| | | | DYSNFYRAF | 207.62 | TPA: helix-turn-helix domain- | HIT79450.1 |
| | | | (SEQ ID No 458) | | containing protein | |
| | | | NYNNFIRYL | 243.95 | ABC transporter ATP-binding | MBO4484513.1 |
| | | | (SEQ ID No 459) | | protein [Lachnospiraceae | |
| | | | | | bacterium] | |
| | | | NYNNFVRYL | 299.5 | ABC transporter ATP-binding | MBR5667449.1 |
| | | | (SEQ ID No 460) | | protein | |
| | | | NYDNFFRML | 715.92 | 23S rRNA (adenine(2503)-C(2))- | MBQ2275888.1 |
| | | | (SEQ ID No 461) | | methyltransferase RlmN | |
| | | | | | [Lachnospiraceae bacterium] | |
| | | | LYNNFERFL | 770 | Xaa-Pro aminopeptidase | SDI89196.1 |
| | | | (SEQ ID No 462) | | [Lachnospiraceae bacterium G41] | |
| | | | AYDEFYRFL | 1278.64 | ATP-dependent DNA helicase RecG | MBQ6105135.1 |
| | | | (SEQ ID No 463) | | | |
| | | | DYSNFNRFL | 1304.83 | fibronectin type III domain- | HIT53773.1 |
| | | | (SEQ ID No 464) | | containing protein | |
| | | | TYNEFYRFY | 2494.72 | translation initiation factor 2 | MCD8119786.1 |
| | | | (SEQ ID No 465) | | [Lachnospiraceae bacterium] | |
| | | | EYDDSYRFL | 3723.4 | phage major capsid protein | WP_097792912.1 |
| | | | (SEQ ID No 466) | | [Faecalibacterium prausnitzii] | |
| | | | DYDNFYRNL | 3769.86 | TPA: hypoxanthine | HIU69252.1 |
| | | | (SEQ ID No 467) | | phosphoribosyltransferase | |
| KM-HN-1 | EYLSLSDKI (SEQ | 249.87 | MYMDLSDKI | 18.82 | extracellular solute-binding protein | RGU04328.1 |
| | ID No 10) | | (SEQ ID No 488) | | | |
| | | | LYLFLSNKI | 31.27 | TPA: carbohydrate ABC transporter | HIR51220.1 |
| | | | (SEQ ID No 489) | | permease | |
| | | | YYLSISDKL | 38.38 | TPA: FAD-binding protein | HIR03062.1 |
| | | | (SEQ ID No 490) | | | |
| | | | CYLSLIDKI | 46.39 | TPA: primosomal protein N | HIU72613.1 |
| | | | (SEQ ID No 491) | | | |
| | | | EYLSLTDQF | 58.87 | type III-A CRISPR-associated protein | RGB96811.1 |
| | | | (SEQ ID No 492) | | Cas10/Csm1 [Faecalibacterium | |
| | | | | | prausnitzii] | |
| | | | KYLSLNDKI | 63.31 | nicotinate-nucleotide | MBQ1487485.1 |
| | | | (SEQ ID No 493) | | adenylyltransferase | |
| | | | | | [Lachnospiraceae bacterium] | |
| | | | QYLTLTDKI | 88.74 | transcriptional regulator, RpiR | EDO62611.1 |
| | | | (SEQ ID No 494) | | family | |
| | | | EYLSMSTKI | 102.61 | DNA methylase [Lachnospiraceae | MBQ5559680.1 |
| | | | (SEQ ID No 495) | | bacterium] | |
| | | | EYLSLSEKI | 113.2 | AAA family ATPase Massilimaliae | WP_077534359.1 |
| | | | (SEQ ID No 496) | | massiliensis | |
| | | | KFLSLFDKI | 140.25 | V-type ATPase 116 kDa subunit | EEG29117.1 |
| | | | (SEQ ID No 497) | | family protein | |
| | | | EYLSFSDKI | 153.16 | NAD(P)H-dependent glycerol-3- | HHZ06911.1 |
| | | | (SEQ ID No 498) | | phosphate dehydrogenase | |
| | | | | | Clostridiales bacterium | |
| | | | KYLSLDDKI | 156.27 | nicotinate-nucleotide | MBQ6638262.1 |
| | | | (SEQ ID No 499) | | adenylyltransferase | |
| | | | | | [Lachnospiraceae bacterium] | |
| | | | KYLSLDDKI | 156.27 | bis(5′-nucleosyl)-tetraphosphatase | MBR3638856.1 |
| | | | (SEQ ID No 500) | | (symmetrical) Yqek | |
| | | | | | [Lachnospiraceae bacterium] | |
| | | | EYLKLTDKI | 160.24 | response regulator | MBQ9503861.1 |
| | | | (SEQ ID No 501) | | [Lachnospiraceae bacterium] | |
| | | | EYLSLSSKI | 168 | GspE/PulE family protein | WP_090091604.1 |
| | | | (SEQ ID No 502) | | Clostridium uliginosum | |
| | | | EYLSLVDKI | 187.93 | tRNA (N6-isopentenyl | WP_059108549.1 |
| | | | (SEQ ID No 503) | | adenosine(37)-C2)- | |
| | | | | | methylthiotransferase MiaB | |
| | | | | | Christensenella hongkongensis | |
| | | | EYISLSDKI | 234.91 | CpaF family protein Clostridiales | MBS6171231.1 |
| | | | (SEQ ID No 504) | | bacterium | |
| | | | EYISLSDKI | 234.91 | CpaF family protein | MCB5920702.1 |
| | | | (SEQ ID No 505) | | [Lachnospiraceae bacterium | |
| | | | | | 210521-DFI.1.105] | |
| | | | EYLSLSDSI | 243.41 | leucine-rich repeat domain- | MBR7044961.1 |
| | | | (SEQ ID No 506) | | containing protein Lachnospiraceae | |
| | | | | | bacterium | |
| | | | EYLSLSDSI | 243.41 | leucine-rich repeat domain- | MBR7044961.1 |
| | | | (SEQ ID No 507) | | containing protein | |
| | | | | | [Lachnospiraceae bacterium] | |
| | | | EYLSLSDKI (SEQ | 249.87 | SMI1/KNR4 family protein | WP_120409454.1 |
| | | | ID No 508) | | Roseburia sp. 1XD42-69 | |
| | | | GYLKLSDKI | 284.35 | anaerobic ribonucleoside- | MBQ9991463.1 |
| | | | (SEQ ID No 509) | | triphosphate reductase | |
| | | | | | [Lachnospiraceae bacterium] | |
| | | | VYLSNSDKV | 398.44 | TPA: prolyl oligopeptidase family | HIU50603.1 |
| | | | (SEQ ID No 510) | | serine peptidase | |
| | | | EYLNINDKI | 440.45 | 2-isopropylmalate synthase | RGQ37783.1 |
| | | | (SEQ ID No 511) | | | |
| | | | EYLSLFDRM | 538.83 | PA: helix-turn-helix transcriptional | HIQ58800.1 |
| | | | (SEQ ID No 512) | | regulator | |
| | | | EYLTLGDKI | 680.99 | flavin reductase family protein | MBQ7765997.1 |
| | | | (SEQ ID No 513) | | [Lachnospiraceae bacterium] | |
| | | | EYLSLKDKL | 1895.38 | Signal transduction histidine kinase | SCY81903.1 |
| | | | (SEQ ID No 514) | | [Lachnospiraceae bacterium | |
| | | | | | XBB2008] | |
| MAGE-A2 | EYLQLVFGI (SEQ | 135.27 | AYLQLVFSI | 28.34 | glycosyltransferase family 2 | MBO6286936.1 |
| | ID No 11) | | (SEQ ID No 539) | | protein Bacilli bacterium | |
| | | | VYLQVVFGI | 31.9 | nicotinamide riboside transporter | WP_133444878.1 |
| | | | (SEQ ID No 540) | | PnuC Macrococcus caseolyticus | |
| | | | SYLQKVFGI | 27.53 | tRNA 4-thiouridine(8) synthase Thil | MBD5494776.1 |
| | | | (SEQ ID No 541) | | [Lachnospiraceae bacterium] | |
| | | | IYLVLVYGI | 39.65 | transposase [Faecalibacterium | WP_173015421.1 |
| | | | (SEQ ID No 542) | | prausnitzii] | |
| | | | EYMQLVAEI | 41.91 | TPA: tRNA (N6-isopentenyl | HIQ79709.1 |
| | | | (SEQ ID No 543) | | adenosine(37)-C2)- | |
| | | | | | methylthiotransferase MiaB | |
| | | | EYLQLIFPI | 44.37 | retron Eco8 family effector | WP_048924608.1 |
| | | | (SEQ ID No 544) | | endonuclease Coprobacillus sp. | |
| | | | | | 8_1_38FAA | |
| | | | QYLHIIFGI | 46.22 | citrate: proton symporter | WP_112148660.1 |
| | | | (SEQ ID No 545) | | [Faecalibacterium prausnitzii] | |
| | | | EYLQIVFNI | 53.19 | DNA alkylation repair protein | MBQ6625054.1 |
| | | | (SEQ ID No 546) | | Clostridia bacterium | |
| | | | EYLQLGFSI | 57.87 | aminoacyl-histidine dipeptidase | MBE5909468.1 |
| | | | (SEQ ID No 547) | | [Lachnospiraceae bacterium] | |
| | | | EYMQRVFGI | 60.4 | DUF89 family protein | MBQ6386375.1 |
| | | | (SEQ ID No 548) | | [Lachnospiraceae bacterium] | |
| | | | EYLELLYGI | 92.27 | site-specific integrase | PNH19151.1 |
| | | | (SEQ ID No 549) | | [Lachnospiraceae bacterium] | |
| | | | VYLQLVRYV | 221.01 | TPA: GntR family transcriptional | k: HIT53822.1 |
| | | | (SEQ ID No 550) | | regulator | |
| | | | EYLQLVTYL | 383.41 | glycosyltransferase | MBT9711503.1 |
| | | | (SEQ ID No 551) | | [Faecalibacterium prausnitzii] | |
| | | | AYLQLKYLY | 645.9 | insulinase family protein | MBR6326373.1 |
| | | | (SEQ ID No 552) | | [Lachnospiraceae bacterium] | |
| | | | EYLQIVENV | 1138.34 | TPA: RIP metalloprotease RseP | HIU50301.1 |
| | | | (SEQ ID No 553) | | | |
| | | | EYLQLVNQV | 1576.42 | CotH kinase family protein | MBP3569136.1 |
| | | | (SEQ ID No 554) | | [Lachnospiraceae bacterium] | |
| | | | EYLQLQRQL | 2014.53 | LacI family transcriptional regulator | MBD9003888.1 |
| | | | (SEQ ID No 555) | | [Faecalibacterium prausnitzii] | |
| | | | EYLELVFNY | 3025.55 | DUF1294 domain-containing | MBO4787423.1 |
| | | | (SEQ ID No 556) | | protein [Lachnospiraceae | |
| | | | | | bacterium] | |
| SAGE | LYATVIHDI (SEQ | 36.15 | YYASLIHDI | 10.8 | HD domain-containing protein | MBO4309970.1 |
| | ID No 12) | | (SEQ ID No 576) | | [Lachnospiraceae bacterium] | |
| | | | IYAAIIHDI (SEQ | 12.64 | HD domain-containing protein | MBE5866694.1 |
| | | | ID No 577) | | [Lachnospiraceae bacterium] | |
| | | | LYQSEIHHI | 14.81 | acetolactate synthase. large | EDO63150.1 |
| | | | (SEQ ID No 578) | | subunit, biosynthetic type | |
| | | | YYATLIHDI | 15.14 | HD domain-containing protein | MBO4775486.1 |
| | | | (SEQ ID No 579) | | Lachnospiraceae bacterium | |
| | | | YYATLIHDI | 15.14 | HD domain-containing protein | MBO4775486.1 |
| | | | (SEQ ID No 580) | | [Lachnospiraceae bacterium] | |
| | | | YYATLVHDI | 17.07 | HD domain-containing protein | MBD5502315.1 |
| | | | (SEQ ID No 581) | | [Lachnospiraceae bacterium] | |
| | | | LYATAIHEI | 18.51 | DUF2663 family protein, partial | WP_115453929.1 |
| | | | (SEQ ID No 582) | | Bacillus piezotolerans | |
| | | | HYANVIHSW | 18.65 | TPA: proline-tRNA ligase | : HIS65767.1 |
| | | | (SEQ ID No 583) | | | |
| | | | GYATVFHKI | 24.9 | N-6 DNA methylase | MBS7060185.1 |
| | | | (SEQ ID No 584) | | [Faecalibacterium prausnitzii] | |
| | | | LYAAVLHEL | 25.51 | peptidase [Faecalibacterium | MBS7082787.1 |
| | | | (SEQ ID No 585) | | prausnitzii] | |
| | | | LYASVLHDI | 27.58 | HD domain-containing | MBO4863242.1 |
| | | | (SEQ ID No 586) | | protein Eubacterium sp.] | |
| | | | LYATVFQDI | 27.99 | ABC transporter ATP-binding | MBD5156035.1 |
| | | | (SEQ ID No 587) | | protein Butyrivibrio sp. | |
| | | | LYATVFQDI | 27.99 | ABC transporter ATP-binding | MBD5538214.1 |
| | | | (SEQ ID No 588) | | protein [Lachnospiraceae | |
| | | | | | bacterium] | |
| | | | LYFTVIHLL | 28.11 | ABC transporter ATP-binding | WP_215696595.1 |
| | | | (SEQ ID No 589) | | protein/permease | |
| | | | | | [Faecalibacterium prausnitzii] | |
| | | | LYAAILHDI | 30.56 | HD domain-containing protein | MBE6011823.1 |
| | | | (SEQ ID No 590) | | [Lachnospiraceae bacterium] | |
| | | | LYLTIIHDI | 32.75 | dihydrofolate | WP_091272910.1 |
| | | | (SEQ ID No 591) | | reductase Alteribacillus | |
| | | | | | persepolensis | |
| | | | LYATVIAEI | 34.51 | ATP-dependent zinc | MBQ6590101.1 |
| | | | (SEQ ID No 592) | | metalloprotease FtsH | |
| | | | | | [Lachnospiraceae bacterium] | |
| | | | AYGTVIHEI | 37 | PucR family transcriptional | MBE5883573.1 |
| | | | (SEQ ID No 593) | | regulator [Lachnospiraceae | |
| | | | | | bacterium] | |
| | | | FYAAVLQDI | 39.33 | arginine-tRNA ligase | EDO60106.1 |
| | | | (SEQ ID No 594) | | | |
| | | | LYSTVFQDI | 40.63 | ABC transporter ATP-binding | MBD5497489.1 |
| | | | (SEQ ID No 595) | | protein [Lachnospiraceae | |
| | | | | | bacterium] | |
| | | | YYETVIHEI | 41.72 | pyrroline-5-carboxylate reductase | BCN32811.1 |
| | | | (SEQ ID No 596) | | [Lachnospiraceae bacterium TB5] | |
| | | | RYVTVIHDL | 44.28 | glycosyltransferase family 4 protein | MBR6850879.1 |
| | | | (SEQ ID No 597) | | [Lachnospiraceae bacterium] | |
| | | | AYQTVIFDI | 47.04 | HAD family hydrolase | RGQ38182.1 |
| | | | (SEQ ID No 598) | | | |
| | | | IYGAVINDI | 62.78 | TPA: InIB B-repeat-containing | HIV17791.1 |
| | | | (SEQ ID No 599) | | protein | |
| | | | AYASVIQDI | 64.27 | DDE transposase [Lachnospiraceae | AVM69455.1 |
| | | | (SEQ ID No 600) | | bacterium oral taxon 500] | |
| | | | FYATVNHDI | 67.57 | L-asparaginase 1 [Lachnospiraceae | GFI65663.1 |
| | | | (SEQ ID No 601) | | bacterium] | |
| | | | NYSAVIHDI | 74.81 | sugar ABC transporter permease | MBR1931959.1 |
| | | | (SEQ ID No 602) | | [Lachnospiraceae bacterium] | |
| | | | LYVAVVQDI | 77.67 | nitroreductase family protein | MBS7028433.1 |
| | | | (SEQ ID No 603) | | [Faecalibacterium prausnitzii] | |
| | | | LYANVIVDI | 88.62 | primosomal protein N' | MCC2149538.1 |
| | | | (SEQ ID No 604) | | [Lachnospiraceae bacterium CLA- | |
| | | | | | AA-H246] | |
| | | | LYASVIVDI | 92.15 | primosomal protein N' | MBR3307355.1 |
| | | | (SEQ ID No 605) | | [Lachnospiraceae bacterium] | |
| | | | LYASVIVDI | 92.15 | primosomal protein N' | MBQ5485557.1 |
| | | | (SEQ ID No 606) | | [Lachnospiraceae bacterium] | |
| | | | LYTTVISAI | 129.48 | DUF368 domain-containing protein | MBS6320402.1 |
| | | | (SEQ ID No 607) | | | |
| | | | LYATVITDI | 139.36 | ATP-dependent zinc | MBR2729061.1 |
| | | | (SEQ ID No 608) | | metalloprotease FtsH | |
| | | | | | [Lachnospiraceae bacterium] | |
| | | | PYVTVIHDL | 222.38 | glycosyltransferase family 4 protein | MBR6381150.1 |
| | | | (SEQ ID No 609) | | [Lachnospiraceae bacterium] | |
| | | | PYVTVIHDL | 222.38 | glycosyltransferase family 4 protein | MBD5527040.1 |
| | | | (SEQ ID No 610) | | [Lachnospiraceae bacterium] | |
| | | | EYAAAVHDI | 282.8 | TPA: sensor histidine kinase | HIR61685.1 |
| | | | (SEQ ID No 611) | | | |
| | | | LYTTVIHDM | 516.88 | glycosyltransferase family 4 protein | MCD7863943.1 |
| | | | (SEQ ID No 612) | | [Lachnospiraceae bacterium] | |
| | | | AYATVISQM | 556.25 | TPA: MATE family efflux transporter HIR59982.1 | |
| | | | (SEQ ID No 613) | | [ | |
| | | | BACTERIOIDES | | | |
| TAA | | | | Aff | | |
| Name | PEPTIDE | Aff (nM) | PEPTIDE | (nM) | specie | Seq ID |
| KM-HN-1 | NYNNFYRFL | 157.45 | MYANFYRFL | 18.89 | glycosyltransferase family 2 | MBK8416245.1 |
| | (SEQ ID No 9) | | (SEQ ID No 468) | | protein [Bacteroidetes | |
| | | | | | bacterium | |
| | | | TYNNFYRFF | 20.38 | O-antigen ligase family | WP_160633064.1 |
| | | | (SEQ ID No 469) | | protein Acidiluteibacter | |
| | | | | | ferrifornacis | |
| | | | RYGNFYRFL | 24.16 | succinylglutamate | WP_134271503.1 |
| | | | (SEQ ID No 470) | | desuccinylase/aspartoacylase | |
| | | | | | family protein Lewinella sp. | |
| | | | | | W8 | |
| | | | NYNSFYRFF | 25.03 | TIGR02757 family protein | MBS6268346.1 |
| | | | (SEQ ID No 471) | | Tannerella sp. | |
| | | | HYQNFYEFL | 26.99 | MULTISPECIES: N-6 DNA | WP_195272477.1 |
| | | | (SEQ ID No 472) | | methylase [Bacteroidales] | |
| | | | KYDNYYRLF | 27.99 | TPA: S9 family peptidase | HIR83490.1 |
| | | | (SEQ ID No 473) | | [Candidatus | |
| | | | | | Cryptobacteroides pullicola] | |
| | | | VYNNFYRLL | 32.39 | TonB-dependent receptor | WP_094414045.1 |
| | | | (SEQ ID No 474) | | [Flavobacterium | |
| | | | | | cyanobacteriorum | |
| | | | NYNNFSIEF | 34.24 | Sensor histidine kinase TodS | KAA6301807.1 |
| | | | | | [Candidatus Ordinivivax | |
| | | | (SEQ ID No 475) | | streblomastigis] | |
| | | | NYNNFSIEF | 34.24 | response regulator | MCD7973646.1 |
| | | | (SEQ ID No 476) | | [Candidatus Azobacteroides | |
| | | | | | sp.] | |
| | | | NYNDFYDKF | 46.12 | TPA: glycogen/starch | HIS22097.1 |
| | | | (SEQ ID No 477) | | synthase [Candidatus | |
| | | | | | Cryptobacteroides | |
| | | | | | intestinipullorum] | |
| | | | KYDNYYRLI | 71.66 | TPA: DPP IV N-terminal | HIS21838.1 |
| | | | (SEQ ID No 478) | | domain-containing protein | |
| | | | | | [Candidatus | |
| | | | | | Cryptobacteroides | |
| | | | | | intestinipullorum] | |
| | | | NYSNIYRFL | 80.98 | glycosyltransferase family 1 | HIN99713.1 |
| | | | (SEQ ID No 479) | | protein Flavobacteriaceae | |
| | | | | | bacterium | |
| | | | NYNNFYSFL | 82.37 | T9SS type A sorting domain- | WP_109853465.1 |
| | | | (SEQ ID No 480) | | containing | |
| | | | | | protein Aquimarina sp. AU58 | |
| | | | NYNRFYDSI | 87.09 | starch synthase catalytic | EDS04678.1 |
| | | | (SEQ ID No 481) | | domain protein [Alistipes | |
| | | | | | putredinis DSM 17216] | |
| | | | NYNNGYRTI | 148.56 | TPA: TonB-dependent | HIT15675.1 |
| | | | (SEQ ID No 482) | | receptor [Candidatus | |
| | | | | | Avimuribaculum pullicola] | |
| | | | DYNGFYANI | 323.08 | S41 family peptidase | UKI39263.1 |
| | | | (SEQ ID No 483) | | [Alistipes putredinis] | |
| | | | HYDNLYSFL | 674.97 | TPA: sugar transferase | HIR38703.1 |
| | | | (SEQ ID No 484) | | [Candidatus Limisoma | |
| | | | | | gallistercoris] | |
| | | | LYDNYYRIL (SEQ | 717.7 | MULTISPECIES: Eco571 | WP_005679986.1 |
| | | | ID No 485) | | restriction-modification | |
| | | | | | methylase domain-containing | |
| | | | | | protein [Bacteroidales] | |
| | | | YYDVFYNDL | 792.26 | TPA: TonB-dependent | HIS22865.1 |
| | | | (SEQ ID No 486) | | receptor [Candidatus | |
| | | | | | Cryptobacteroides | |
| | | | | | intestinipullorum] | |
| | | | LYNTFYHFY | 934.97 | transposase [Candidatus | GAP72185.1 |
| | | | (SEQ ID No 487) | | Symbiothrix dinenymphae] | |
| KM-HN-1 | EYLSLSDKI (SEQ | 249.87 | VYLSISEKI | 11.19 | tetratricopeptide repeat | MBR3831564.1 |
| | ID No 10) | | (SEQ ID No 515) | | protein Muribaculaceae | |
| | | | YYLELSEKI | 13.15 | TPA: PepSY-like domain- | HIW86655.1 |
| | | | (SEQ ID No 516) | | containing protein | |
| | | | | | [Candidatus Onthomorpha | |
| | | | | | intestinigallinarum] | |
| | | | YYLALSDKI | 17.52 | LPS-assembly protein LptD | MBP3767014.1 |
| | | | (SEQ ID No 517) | | Prevotella sp. | |
| | | | SYLSLANNI | 18.35 | toprim domain-containing | WP_215721770.1 |
| | | | (SEQ ID No 518) | | protein [Alistipes putredinis] | |
| | | | RYLSLSDKI | 24.29 | polysaccharide pyruvyl | MBP3566537.1 |
| | | | (SEQ ID No 519) | | transferase family protein | |
| | | | | | Paraprevotella sp. | |
| | | | IYLSLADKI | 25.89 | histidine ammonia- | MBN2349226.1 |
| | | | (SEQ ID No 520) | | lyase Bacteroidales bacterium | |
| | | | RYLALSDKI | 26.76 | MULTISPECIES: | WP_117507904.1 |
| | | | (SEQ ID No 521) | | polysaccharide pyruvyl | |
| | | | | | transferase family protein | |
| | | | | | Alistipes] | |
| | | | KYLSLTDEI 29.18 | | TPA: dihydrofolate reductase | HIS23517.1 |
| | | | (SEQ ID No 522) | | [Candidatus | |
| | | | | | Cryptobacteroides | |
| | | | | | intestinipullorum] | |
| | | | KYLSLADKI | 35.18 | TPA: NAD(P)/FAD-dependent | HBG53681.1 |
| | | | (SEQ ID No 523) | | oxidoreductase Rikenellaceae | |
| | | | | | bacterium | |
| | | | NYLSLSEKI 38.78 | | GHKL domain-containing | MBT5932717.1 |
| | | | (SEQ ID No 524) | | protein [Flavobacteriales | |
| | | | | | bacterium | |
| | | | QYLSLSNKI | 45.22 | T9SS type A sorting domain- | MBN1133131.1 |
| | | | (SEQ ID No 525) | | containing protein | |
| | | | | | Bacteroidales bacterium | |
| | | | FYLSLNDKI | 73.36 | FAD-binding oxidoreductase | WP_077374390.1 |
| | | | (SEQ ID No 526) | | FAD-binding oxidoreductase | |
| | | | EYLSLSQKI | 73.82 | CAAX amino terminal | OPZ03153.1 |
| | | | (SEQ ID No 527) | | protease self- | |
| | | | | | immunity Bacteroidetes | |
| | | | | | bacterium ADurb.BinA395 | |
| | | | EYLSISNKI | 96.92 | efflux RND transporter | MBK9212880.1 |
| | | | (SEQ ID No 528) | | periplasmic adaptor subunit | |
| | | | | | Saprospiraceae bacterium | |
| | | | HYLSLSDKI | 102.11 | 16S rRNA (adenine(1518)- | MBS1488621.1 |
| | | | (SEQ ID No 529) | | N(6)/adenine(1519)-N(6))- | |
| | | | | | dimethyltransferase RsmA | |
| | | | | | [Bacteroidetes bacterium | |
| | | | FYLDLSDII | 109.3 | AraC family transriptional | MCD7974127.1 |
| | | | (SEQ ID No 530) | | regulator [Candidatus | |
| | | | | | Azobacteroides sp.] | |
| | | | EYLQLTTKI | 132.29 | toprim domain-containing | HIT15593.1 |
| | | | (SEQ ID No 531) | | protein [Candidatus | |
| | | | | | Avimuribaculum pullicola] | |
| | | | EYISLIDEI | 153.5 | nicotinate (nicotinamide) | UKI39005.1 |
| | | | (SEQ ID No 532) | | nucleotide | |
| | | | | | adenylyltransferase [Allstipes | |
| | | | | | putredinis] | |
| | | | EYLWLADKI | 175.98 | MobC family plasmid | WP_234222759.1 |
| | | | (SEQ ID No 533) | | mobilization relaxosome | |
| | | | | | protein [Phocaeicola vulgatus] | |
| | | | DYLSLSDIF | 183.23 | MULTISPECIES: | WP_057279431.1 |
| | | | (SEQ ID No 534) | | glycosyltransferase | |
| | | | | | [Phocaeicola] | |
| | | | EYLSISRII | 207.14 | MULTISPECIES: Fic family | WP_141405227.1 |
| | | | (SEQ ID No 535) | | protein [Alistipes] | |
| | | | EYLRISDEI | 213.28 | 546 family peptidase | HIQ84544.1 |
| | | | (SEQ ID No 536) | | [Candidatus | |
| | | | | | Cryptobacteroides | |
| | | | | | excrementigallinarum] | |
| | | | EYLSLKNII | 481.6 | TPA: ABC transporter | HIW86671.1 |
| | | | (SEQ ID No 537) | | permease [Candidatus | |
| | | | | | Onthomorpha | |
| | | | | | intestinigallinarum] | |
| | | | EYYLSLVVEV | 1394.46 | TIGR01212 family radical SAM | MBS6651286.1 |
| | | | (SEQ ID No 538) | | protein [Alistipes putredinis] | |
| MAGE-A2 | EYLQLVFGI (SEQ | 135.27 | TYMQMVFGF | 5.53 | Sodium/glucose cotransporter | KAA6302458.1 |
| | ID No 11) | | (SEQ ID No 557) | | [Candidatus Ordinivivax | |
| | | | | | streblomastigis] | |
| | | | RYLHLVFGI | 11.45 | histidine kinase Arsenicibacter | WP_071503703.1 |
| | | | (SEQ ID No 558) | | rosenii | |
| | | | EYLQLFHQI | 22.61 | AAA ATPase [Candidatus | GAP71734.1 |
| | | | (SEQ ID No 559) | | Symbiothrix dinenymphae] | |
| | | | EYAPLVFEI | 34.46 | TPA: outer membrane beta- | HIT15790.1 |
| | | | (SEQ ID No 560) | | barrel protein [Candidatus | |
| | | | | | Avimuribaculum pullicola] | |
| | | | YYLQLVDKL | 42.03 | TPA: oxaloacetate | HIR83440.1 |
| | | | (SEQ ID No 561) | | decarboxylase [Candidatus | |
| | | | | | Cryptobacteroides pullicola] | |
| | | | LYLQLVFAF | 47.39 | oligosaccharide repeat unit | WP_016270428.1 |
| | | | (SEQ ID No 562) | | polymerase [Phocaeicola | |
| | | | | | vulgatus] | |
| | | | EYLQLVYGF | 50.1 | beta-N-acetylhexosaminidase, | RYZ30368.1 |
| | | | (SEQ ID No 563) | | partial Chitinophagaceae | |
| | | | | | bacterium | |
| | | | KYLQTVFGL | 59.91 | hexuronate transporter | BAG83909.1 |
| | | | (SEQ ID No 564) | | [Candidatus Azobacteroides | |
| | | | | | pseudotrichonymphae | |
| | | | | | genomovar. CFP2] | |
| | | | EYLQMAFGI | 84.53 | phage gp6-like head-tail | MBQ6688332.1 |
| | | | (SEQ ID No 565) | | connector protein | |
| | | | | | Bacteroidales bacterium] | |
| | | | EYLQLTTKI | 132.29 | TPA: toprim domain- | HIT15593.1 |
| | | | (SEQ ID No 566) | | containing protein | |
| | | | | | [Candidatus Avimuribaculum | |
| | | | | | pullicola] | |
| | | | EYLQSAFGI | 149.89 | nitrogenase molybdenum- | KAA6301763.1 |
| | | | (SEQ ID No 567) | | iron protein NifN [Candidatus | |
| | | | | | Ordinivivax streblomastigis] | |
| | | | EYLTLVCGF | 325.48 | metal-binding protein | OKY93250.1 |
| | | | (SEQ ID No 568) | | [Alistipes putredinis] | |
| | | | EYVQMIFSM | 397.78 | recombinase family protein | MBS5322006.1 |
| | | | (SEQ ID No 569) | | [Alistipes putredinis] | |
| | | | AFLQLVETI | 442.61 | Spore coat protein SA | KAA6300759.1 |
| | | | (SEQ ID No 570) | | [Candidatus Ordinivivax | |
| | | | | | streblomastigis] | |
| | | | EYLQQVIDI | 684.22 | AAA family ATPase | MCD7973596.1 |
| | | | (SEQ ID No 571) | | [Candidatus Azobacteroides | |
| | | | | | sp.] | |
| | | | EYLSLVVEV | 1394.46 | TIGR01212 family radical SAM | MBS6651286.1 |
| | | | (SEQ ID No 572) | | protein [Alistipes putredinis] | |
| | | | EYLQAVREV | 1772.63 | NADP-specific glutamate | WP_004330097.1 |
| | | | (SEQ ID No 573) | | dehydrogenase [Alistipes | |
| | | | | | putredinis] | |
| | | | EYLQLELRM | 2767.63 | TolC family protein | |
| | | | (SEQ ID No 574) | | [Candidatus | |
| | | | | | Cryptobacteroides pullicola] | |
| | | | EYLQDVFHY | 4748.83 | TPA: GNAT family N- | HIR82591.1 |
| | | | (SEQ ID No 575) | | acetyltransferase [Candidatus | |
| | | | | | Cryptobacteroides pullicola] | |
| SAGE | LYATVIHDI (SEQ | 36.15 | KYAAVIEEI (SEQ | 20.8 | preprotein translocase | WP_055432820.1 |
| | ID No 12) | | ID No 614) | | subunit SecA [Candidatus | |
| | | | | | Symbiothrix dinenymphae] | |
| | | | LYSTVIHEI | 21.9 | T9SS type A sorting domain- | WP_204530868.1 |
| | | | (SEQ ID No 615) | | containing | |
| | | | | | protein Chryseobacterium sp. | |
| | | | | | JUb44 | |
| | | | MYALSIHDI | 33.09 | putative mycofactocin | KAA6303530.1 |
| | | | (SEQ ID No 616) | | biosynthesis | |
| | | | | | glycosyltransferase MftF | |
| | | | | | [Candidatus Ordinivivax | |
| | | | | | streblomastigis] | |
| | | | VYPLVIADI | 36.44 | T9SS type A sorting domain- | MCD7971608.1 |
| | | | (SEQ ID No 617) | | containing protein | |
| | | | | | [Candidatus Azobacteroides | |
| | | | | | sp.] | |
| | | | LYAKVIEDW | 46.82 | TPA: SAM-dependent DNA | HIQ85501.1 |
| | | | (SEQ ID No 618) | | methyltransferase | |
| | | | | | [Candidatus | |
| | | | | | Cryptobacteroides | |
| | | | | | excrementigallinarum] | |
| | | | PYATLIFDI | 55.05 | FKBP-type peptidyl-prolyl cis- | HIQ85112.1 |
| | | | (SEQ ID No 619) | | trans isomerase fklB | |
| | | | | | [Candidatus Symbiothrix | |
| | | | | | dinenymphae] | |
| | | | LYAAVIWFL | 77.09 | TPA: sulfatase-like | HIU38446.1 |
| | | | (SEQ ID No 620) | | hydrolase/transferase. partial | |
| | | | | | [Candidatus Limisoma | |
| | | | | | intestinavium] | |
| | | | RYATVIGIL | 87.45 | rod shape-determining | UKI39671.1 |
| | | | (SEQ ID No 621) | | protein [Alistipes putredinis] | |
| | | | LYASLIHDL | 88.91 | DarT ssDNA thymidine ADP- | WP_172774650.1 |
| | | | (SEQ ID No 622) | | ribosyltransferase family | |
| | | | | | protein [Phocaeicola vulgatus] | |
| | | | LYATLIMVL | 115.97 | Multidrug export protein | KAA6301466.1 |
| | | | (SEQ ID No 623) | | MepA [Candidatus Ordinivivax | |
| | | | | | streblomastigis] | |
| | | | LYATLNHEL | 122.79 | TPA: flippase-like domain- | HIQ84490.1 |
| | | | (SEQ ID No 624) | | containing protein | |
| | | | | | [Candidatus | |
| | | | | | Cryptobacteroides | |
| | | | | | excrementigallinarum] | |
| | | | LYATVGFGL | 151.95 | TPA: PorT family protein | HIR36767.1 |
| | | | (SEQ ID No 625) | | [Candidatus Limisoma | |
| | | | | | gallistercoris] | |
| | | | YTTVISDI | 187.18 | DEAD/DEAH box helicase | WP_117853018.1 |
| | | | (SEQ ID No 626) | | [Phocaeicola vulgatus] | |
| | | | LYATVYILV | 206.76 | ABC transporter permease | MCD7972560.1 |
| | | | (SEQ ID No 627) | | [Candidatus Azobacteroides | |
| | | | | | sp.] | |
| | | | LYATVLVFL | 232.9 | phosphatase PAP2 family | HIR82641.1 |
| | | | (SEQ ID No 628) | | protein [Candidatus | |
| | | | | | Cryptobacteroides pullicola] | |
| | | | LYATVEQNL | 299.76 | TPA: BACON domain- | HIR37436.1 |
| | | | (SEQ ID No 629) | | containing protein | |
| | | | | | [Candidatus Limisoma | |
| | | | | | gallistercoris] | |
| | | | YDNVIHDI | 902.43 | 1-deoxy-D-xylulose 5- | ALK82916.1 |
| | | | (SEQ ID No 630) | | phosphate synthase | |
| | | | | | [Phocaeicola vulgatus] | |
| | | | AYDNVIHDI | 902.43 | 1-deoxy-D-xylulose-5- | KAA6300193.1 |
| | | | (SEQ ID No 631) | | phosphate synthase | |
| | | | | | [Candidatus Ordinivivax | |
| | | | | | streblomastigis] | |

Concerning the tumor-specific TAAs, among others, high levels of homology were found for antigens of the MAGE family (Melanoma Associated Gene protein family), in particular MAGE-A10, MAGE-A3, MAGE-C1 and MAGE-C2. Interestingly, two distinct MAGE-A3 TAAs (FLWGPRALV and KVAELVHFL) were found to share homology with bacteria species of the two phyla.

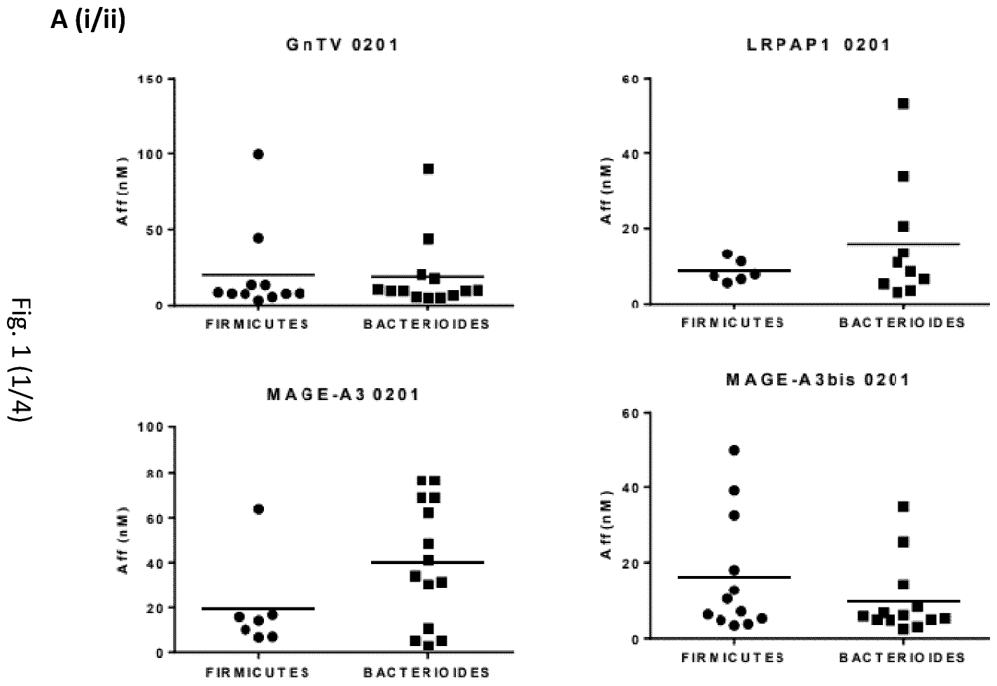

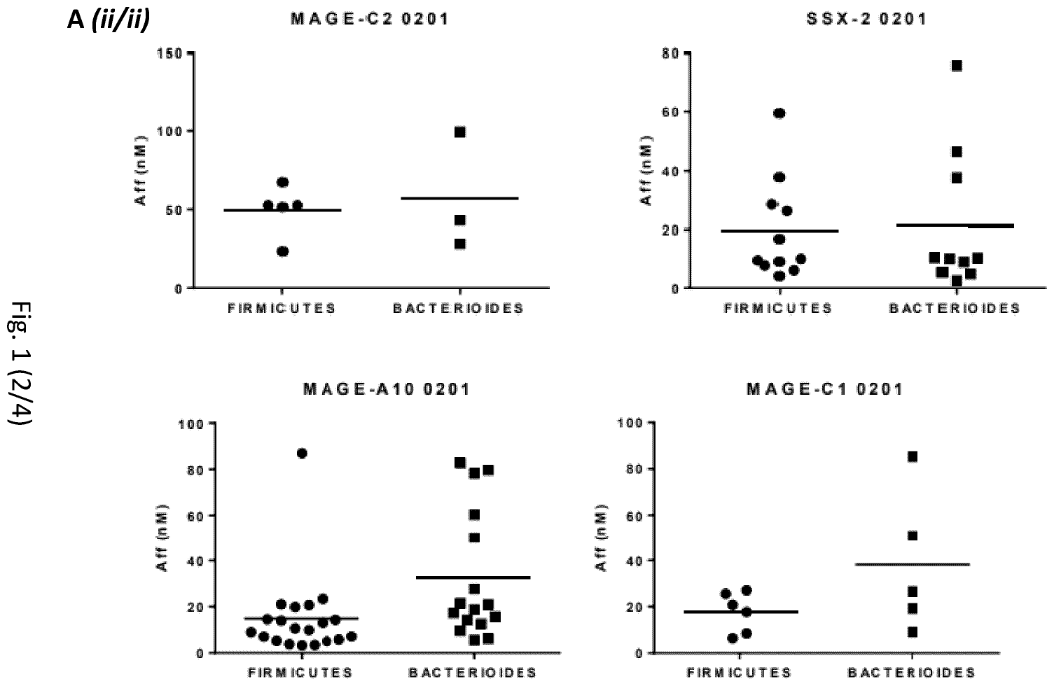

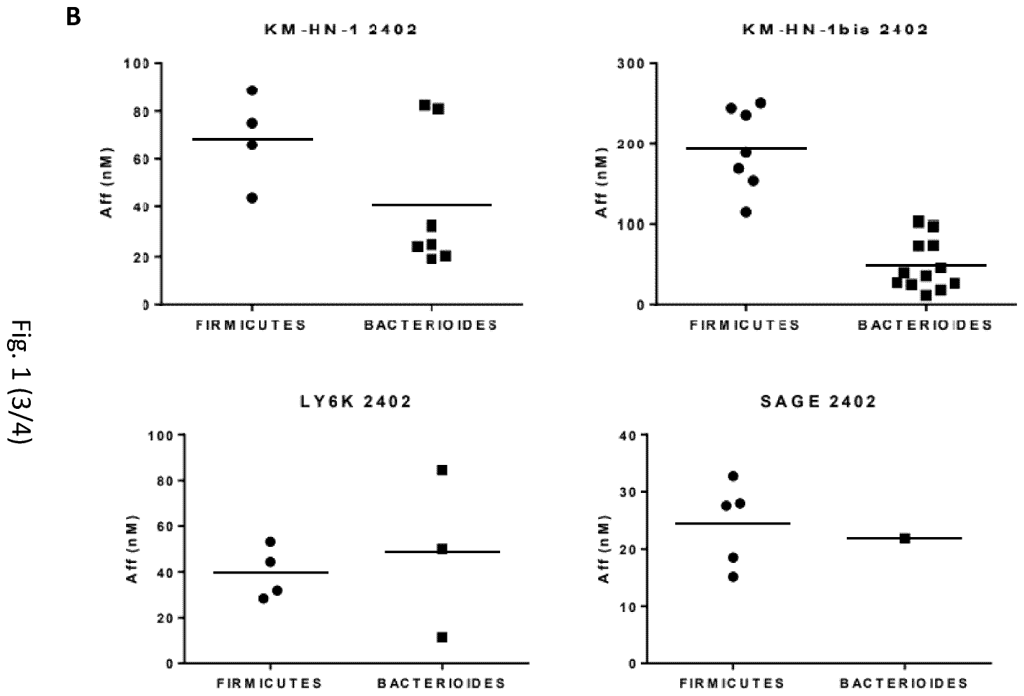

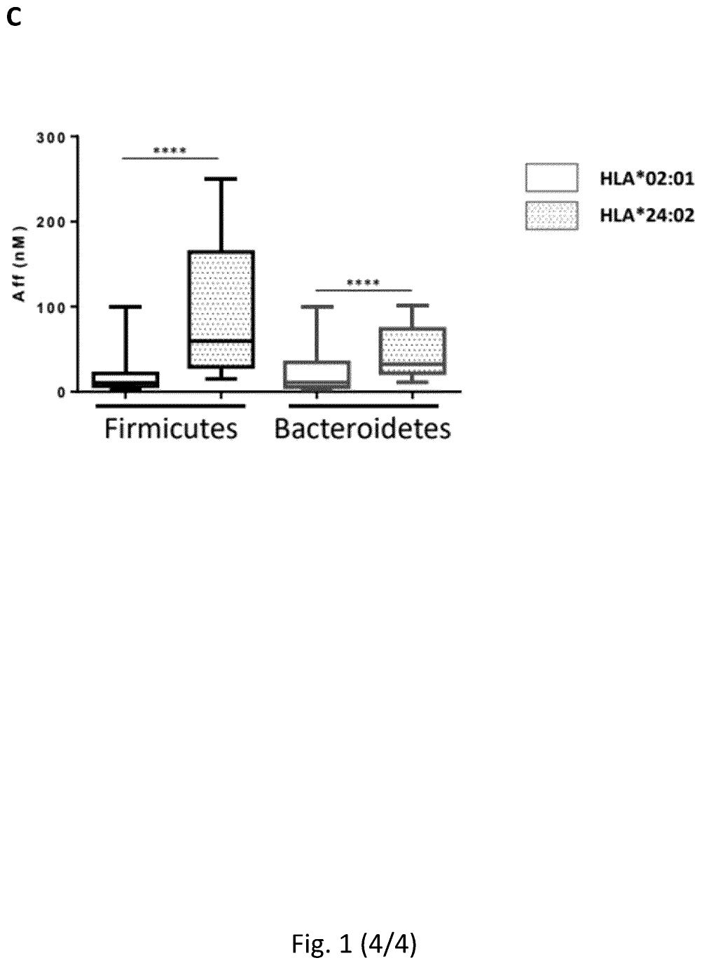

Overall, for each of the analyzed TAAs, the homology was found with several peptides derived from different species in the two phyla with a very high affinity (<100 nM). In particular, the average affinity was 34.4 nM and 30.0 nM for the peptides derived from Firmicutes and Bacteroidetes, respectively (FIGS. 1A and B). Interestingly, the average affinity values were significantly higher for the bacterial peptides restricted for HLA*02:01 (18.8 and 26.6 nM, respectively) than for HLA*24:02 (95.2 and 44.48 nM, respectively) (FIG. 1 C). Affinity values were calculated as described in Ragone C et al., PMID: 34049932.

The alignment of representative TAAs with homologous bacterial peptides shows that the amino acid residues at each position are mostly identical or of the same class (conservative substitution) (Table 3).

TABLE 3
Alignment of TAAs with the homologous bacterial
peptides. The phyla (Firmicutes or Bacteroidetes)
is indicated on top of each box; dot = identical
aa residue; conservative aa substitution are
indicated in black; non-conservative aa substitu-
tion are indicated underlined and in italicus.
FIRMICUTES

| GnTV | V | L | P | D | V | F | I | R | V |
|---|---|---|---|---|---|---|---|---|---|
| MBD5555280.1 | Y | . | . | . | I | . | . | . | . |
| MBP1547557.1 | R | . | . | . | I | . | . | . | . |
| MBP0955392.1 | . | . | . | . | . | . | . | . | . |
| NLO48847.1 | . | . | . | . | I | . | . | K | . |
| MBQ2781239.1 | . | . | . | . | . | . | L | . | . |
| WP_191543324.1 | . | . | . | E | . | . | V | . | . |
| MBQ5332073.1 | . | . | . | E | . | . | . | K | . |
| MBE5970738.1 | I | . | . | . | . | . | . | . | L |
| WP_073279788.1 | E | . | . | . | . | . | . | . | C |
| WP_002573979.1 | N | . | . | . | . | . | . | . | I |
| HEK51933.1 | F | . | . | . | . | . | . | . | C |

BACTEROIDETES

| GnTV | V | L | P | D | V | F | I | R | V |
|---|---|---|---|---|---|---|---|---|---|
| WP_118465647.1 | L | . | S | . | . | . | . | . | . |
| MBR6655791.1 | F | . | . | . | I | . | . | . | I |
| MBR1962375.1 | F | . | . | . | . | . | . | . | I |
| PGH37641.1 | I | . | Q | . | . | . | . | . | . |
| MBM3443229.1 | G | . | . | . | . | . | L | . | . |
| WP_136842267.1 | G | . | . | . | . | . | L | . | . |
| WP_090992641.1 | G | . | . | . | . | . | L | . | . |
| CUN36400.1 | . | . | Q | E | . | . | . | . | . |
| GFD90141.1 | . | . | . | . | T | . | . | . | . |
| MBO7397429.1 | . | . | . | . | . | . | . | G | L |
| RCU43886.1 | . | . | . | . | . | . | . | A | L |
| WP_204246478.1 | . | . | . | . | . | . | V | . | A |
| MBP7641748.1 | . | . | . | . | A | . | . | . | A |

FIRMICUTES

| LRPAP1 | K | V | L | E | Y | V | I | K | V |
|---|---|---|---|---|---|---|---|---|---|
| NLI26328.1 | . | . | . | . | . | . | . | R | . |
| WP_077853732.1 | . | . | . | . | . | . | A | . | . |
| WP_206808822.1 | . | . | . | . | . | . | . | D | . |
| WP_186300960.1 | . | . | . | . | . | . | T | . | . |
| RKW64949.1 | . | . | . | . | . | A | . | . | . |
| MBC7087093.1 | . | . | . | . | . | I | . | . | I |

BACTEROIDETES

| LRPAP1 | K | V | L | E | Y | V | I | K | V |
|---|---|---|---|---|---|---|---|---|---|
| WP_215235361.1 | A | L | . | . | . | . | . | . | . |
| WP_115829840.1 | H | L | . | . | . | . | . | . | . |
| WP_130140291.1 | R | I | . | . | . | . | . | . | . |
| MBK6621957.1 | . | . | . | . | . | . | A | . | . |
| WP_173416227.1 | A | . | . | . | . | . | V | . | . |
| WP_109650090.1 | . | . | . | . | . | . | E | . | . |
| HHE65310.1 | . | . | . | . | . | C | . | . | . |
| MBP5995608.1 | N | . | . | . | . | . | V | . | . |
| MBK5280326.1 | L | . | . | . | . | . | . | . | L |
| MBO7562654.1 | . | . | . | . | . | . | K | . | . |

FIRMICUTES

| MAGE-A10 | G | L | Y | D | G | M | E | H | L |
|---|---|---|---|---|---|---|---|---|---|
| MBQ7053643.1 | Y | . | . | . | D | . | . | . | . |
| WP_012101781.1 | . | . | . | . | . | . | . | Y | I |
| WP_185142326.1 | . | . | . | . | . | V | . | . | V |
| WP_117516162.1 | K | . | . | . | . | I | . | . | . |
| EDS01593.1 | M | . | . | . | . | . | . | R | . |
| WP_132012393.1 | R | . | . | . | . | L | . | . | . |
| CAC8447084.1 | . | . | . | E | . | . | N | . | . |
| WP_008590666.1 | A | . | . | . | . | . | . | Q | . |
| MBQ2769940.1 | . | . | F | . | S | . | . | . | . |
| MBQ7360485.1 | Q | . | . | N | . | . | . | . | V |
| HHT96763.1 | . | . | . | . | V | . | . | . | I |
| MBQ5522151.1 | L | . | . | A | . | . | . | . | . |
| MBR0350109.1 | K | . | . | . | . | . | . | D | . |
| MBQ3010667.1 | . | . | . | E | T | . | . | . | . |
| WP_173629024.1 | A | . | . | . | T | . | . | . | . |
| WP_206458099.1 | . | . | . | S | . | . | . | . | I |
| WP_063441043.1 | . | . | . | . | . | . | . | F | T |
| WP_095131333.1 | N | . | . | . | N | . | . | . | . |
| WP_098095089.1 | . | . | . | E | . | . | K | . | . |
| WP_212975324.1 | . | . | . | . | . | . | . | V | T |

BACTEROIDETES

| MAGE-A10 | G | L | Y | D | G | M | E | H | L |
|---|---|---|---|---|---|---|---|---|---|
| RYZ62962.1 | . | M | . | . | . | L | . | . | . |
| WP_046368370.1 | . | . | . | . | . | L | . | N | . |
| MBN1398196.1 | . | . | . | . | . | . | . | L | A |
| NCA71030.1 | V | . | . | . | N | . | . | . | . |
| WP_089782056.1 | . | . | . | . | E | . | . | Q | . |
| WP_135531157.1 | . | . | . | . | . | . | . | K | A |
| MBQ2520985.1 | A | . | . | . | . | . | . | D | . |
| WP_194101443.1 | . | . | F | . | . | . | K | . | . |
| WP_071843189.1 | A | . | . | N | . | . | D | . | . |
| WP_132555660.1 | . | . | . | . | . | . | R | . | . |
| WP_188752688.1 | F | V | . | . | . | T | . | . | A |
| MBF8294546.1 | E | . | . | . | . | L | . | . | . |
| WP_027076713.1 | A | I | . | . | . | . | . | . | A |
| WP_188752688.1 | V | T | . | . | . | . | . | . | V |
| WP_188752688.1 | F | V | . | . | . | L | . | . | T |
| MBC8082572.1 | . | . | . | . | . | . | . | K | T |

FIRMICUTES

| MAGE-A3 | K | V | A | E | L | V | H | F | L |
|---|---|---|---|---|---|---|---|---|---|
| MBP8691684.1 | . | I | . | . | . | . | . | . | . |
| MBP7221824.1 | . | I | . | . | M | . | . | . | . |
| WP_054028593.1 | A | I | . | . | . | . | . | . | . |
| MBQ3503822.1 | R | . | . | . | . | I | . | . | . |
| WP_035147891.1 | . | I | T | . | . | . | . | . | . |
| WP_143846559.1 | H | I | . | . | . | . | . | . | . |
| WP_188696003.1 | Q | . | . | . | . | . | . | . | . |

BACTEROIDETES

| MAGE-A3 | K | V | A | E | L | V | H | F | L |
|---|---|---|---|---|---|---|---|---|---|
| MBM3426064.1 | V | L | . | . | . | . | . | . | . |
| MBS1647326.1 | . | . | . | . | F | I | . | . | . |
| WP_207362944.1 | . | . | . | . | F | I | . | . | . |
| WP_078062927.1 | . | . | . | . | . | . | . | H | V |
| WP_169607473.1 | . | . | T | . | . | . | . | . | . |
| NBR83089.1 | . | . | . | A | . | . | . | . | . |
| WP_126321206.1 | . | . | . | . | . | . | S | . | . |
| WP_095918246.1 | . | . | . | . | . | . | N | . | . |
| NQX86506.1 | Q | . | . | . | . | I | . | . | . |
| MBQ3673518.1 | A | T | . | . | . | . | . | . | . |
| NLC86325.1 | . | . | . | D | . | . | . | . | A |
| HBT85187.1 | . | . | . | D | . | . | . | . | A |
| MBC7417057.1 | . | . | S | . | . | . | N | . | . |
| WP_202104029.1 | . | . | S | . | . | . | N | . | . |

FIRMICUTES

| MAGE-A3 | F | L | W | G | P | R | A | L | V |
|---|---|---|---|---|---|---|---|---|---|
| MBQ1820230.1 | . | . | . | A | . | I | . | . | . |
| MBQ6708923.1 | . | . | . | . | . | F | S | . | . |
| NLY91350.1 | Y | . | . | . | L | . | . | . | . |
| HHY09163.1 | . | . | . | . | G | . | . | . | L |
| MBC8587361.1 | . | . | . | . | . | F | . | . | I |
| WP_070611851.1 | . | . | . | . | . | A | . | . | I |
| MBQ6563626.1 | . | . | . | N | . | . | . | . | L |
| MBR4261781.1 | S | . | . | . | H | . | . | . | . |
| WP_161846669.1 | . | . | . | . | S | . | G | . | . |
| WP_209615324.1 | . | . | . | A | . | . | . | K | I |
| WP_113724721.1 | . | . | . | . | . | . | . | K | I |
| WP_203627266.1 | . | . | . | . | . | . | Q | . | A |

BACTEROIDETES

| MAGE-A3 | F | L | W | G | P | R | A | L | V |
|---|---|---|---|---|---|---|---|---|---|
| MBQ8420774.1 | . | . | . | . | S | I | . | . | . |
| MBF1567568.1 | . | . | . | E | S | . | . | . | . |
| TAH08235.1 | . | . | . | . | . | H | D | . | . |
| WP_204474554.1 | . | . | . | N | K | . | . | . | . |
| MBN2774199.1 | . | . | . | N | K | . | . | . | . |
| MBE7199410.1 | . | . | . | . | . | I | G | . | . |
| WP_068823941.1 | R | . | . | . | . | I | . | . | . |
| MBL6865741.1 | . | M | . | . | . | . | . | E | . |
| MBL0883181.1 | . | . | . | . | . | K | . | . | . |
| WP_137344335.1 | . | . | . | . | L | . | . | . | I |
| MBC8007764.1 | . | . | . | . | R | . | . | V | . |
| NWK64964.1 | L | . | . | . | . | K | . | . | . |
| RQP14817.1 | Q | . | . | . | A | . | . | . | . |

FIRMICUTES

| MAGE-C1 | K | V | V | E | F | L | A | M | L |
|---|---|---|---|---|---|---|---|---|---|
| TLS53416.1 | R | L | . | . | . | . | . | . | . |
| MBR5011159.1 | . | I | L | . | . | . | . | . | . |
| WP_105977819.1 | . | . | A | D | . | . | . | . | . |
| HBP64347.1 | . | . | I | . | . | . | S | . | . |
| MBO5479965.1 | . | . | . | . | . | . | . | . | . |
| WP_092482186.1 | G | . | I | . | . | . | . | . | . |
| WP_092482186.1 | G | . | I | . | . | . | . | . | . |

BACTEROIDETES

| MAGE-C1 | K | V | V | E | F | L | A | M | L |
|---|---|---|---|---|---|---|---|---|---|
| WP_111611765.1 | . | . | I | . | . | . | . | L | . |
| WP_210489586.1 | . | . | . | D | . | . | . | L | . |
| QCX40738.1 | I | . | L | . | . | . | . | . | . |
| MBL7846776.1 | T | . | L | . | . | . | . | . | . |
| QTN38443.1 | Q | . | A | . | . | . | . | . | . |

FIRMICUTES

| MAGE-C2 | A | L | K | D | V | E | E | R | V |
|---|---|---|---|---|---|---|---|---|---|
| WP_125553189.1 | . | . | S | . | . | . | . | . | . |
| MBQ7934778.1 | M | . | . | . | I | . | . | . | . |
| MBQ7202456.1 | . | . | . | . | . | . | . | P | . |
| MBQ6558180.1 | . | . | . | . | . | . | . | P | . |
| WP_053430754.1 | M | . | . | E | . | . | . | . | . |

BACTEROIDETES

| MAGE-C2 | A | L | K | D | V | E | E | R | V |
|---|---|---|---|---|---|---|---|---|---|
| WP_076782989.1 | Y | . | . | . | . | . | . | . | . |
| MBQ1773637.1 | . | . | D | . | . | . | . | . | . |
| WP_188370161.1 | I | . | . | . | I | . | . | . | . |

FIRMICUTES

| SSX-2 | K | A | S | E | K | I | F | Y | V |
|---|---|---|---|---|---|---|---|---|---|
| WP_193137167.1 | Y | . | . | . | . | M | . | . | . |
| OJU08501.1 | G | V | . | . | . | . | . | . | . |
| HHY18381.1 | A | N | . | . | . | . | . | . | . |
| MBQ6843512.1 | . | . | A | D | . | . | . | . | . |
| WP_039861470.1 | D | . | T | . | . | . | . | . | . |
| MBR2037171.1 | . | . | A | . | . | F | . | . | . |
| MBQ8518875.1 | . | . | . | D | . | . | . | . | . |
| WP_057897585.1 | I | . | G | . | . | . | . | . | . |
| WP_084053470.1 | . | . | N | . | . | . | Y | . | . |
| WP_091273761.1 | S | . | . | . | . | . | Y | . | . |
| WP_098398638.1 | G | S | . | . | . | . | . | . | . |

BACTEROIDETES

| SSX-2 | K | A | S | E | K | I | F | Y | V |
|---|---|---|---|---|---|---|---|---|---|
| RYE15078.1 | Y | T | . | . | . | . | . | . | . |
| MBQ6038980.1 | . | . | Y | . | . | . | . | . | . |
| MBK9284067.1 | . | V | T | . | . | . | . | . | . |
| HCQ15094.1 | . | T | . | . | . | V | . | . | . |
| MBQ9583024.1 | . | . | A | . | . | V | . | . | . |
| NBP69626.1 | . | T | T | . | . | . | . | . | . |
| WP_196828382.1 | . | V | . | . | . | . | . | . | L |
| MBP1646831.1 | G | . | . | . | R | . | . | . | . |
| WP_132989163.1 | V | . | . | . | . | . | . | F | . |
| WP_027124754.1 | V | . | . | . | . | T | . | . | . |

FIRMICUTES

| KM-HN-1 | N | Y | N | N | F | Y | R | F | L |
|---|---|---|---|---|---|---|---|---|---|
| WP_155705275.1 | R | . | . | G | . | . | . | . | . |
| WP_154750440.1 | K | . | . | . | Y | . | . | . | . |
| WP_191684926.1 | . | . | . | S | . | . | S | . | . |
| WP_088223547.1 | . | . | . | . | . | . | V | . | . |

BACTEROIDETES

| KM-HN-1 | N | Y | N | N | F | Y | R | F | L |
|---|---|---|---|---|---|---|---|---|---|
| MBK8416245.1 | M | . | A | . | . | . | . | . | . |
| WP_160633064.1 | T | . | . | . | . | . | . | . | F |
| WP_134271503.1 | R | . | G | . | . | . | . | . | . |
| MBS6268346.1 | . | . | . | S | . | . | . | . | F |
| WP_094414045.1 | V | . | . | . | . | . | . | L | . |
| HIN99713.1 | . | . | S | . | I | . | . | . | . |
| WP_109853465.1 | . | . | . | . | . | . | S | . | . |

FIRMICUTES

| KM-HN-1 | E | Y | L | S | L | S | D | K | I |
|---|---|---|---|---|---|---|---|---|---|
| WP_077534359.1 | . | . | . | . | . | . | E | . | . |
| HHZ06911.1 | . | . | . | . | F | . | . | . | . |
| WP_090091604.1 | . | . | . | . | . | . | S | . | . |
| WP_059108549.1 | . | . | . | . | . | V | . | . | . |
| MBS6171231.1 | . | . | I | . | . | . | . | . | . |
| MBR7044961.1 | . | . | . | . | . | . | . | S | . |
| WP_120409454.1 | . | . | . | . | . | . | . | . | . |

BACTEROIDETES

| KM-HN-1 | E | Y | L | S | L | S | D | K | I |
|---|---|---|---|---|---|---|---|---|---|
| MBR3831564.1 | V | . | . | . | I | . | E | . | . |
| MBP3767014.1 | Y | . | . | A | . | . | . | . | . |
| MBP3566537.1 | R | . | . | . | . | . | . | . | . |
| MBN2349226.1 | I | . | . | . | . | A | . | . | . |
| WP_117507904.1 | R | . | . | A | . | . | . | . | . |
| HBG53681.1 | K | . | . | . | . | A | . | . | . |
| MBT5932717.1 | N | . | . | . | . | . | E | . | . |
| MBN1133131.1 | Q | . | . | . | . | . | N | . | . |
| WP_077374390.1 | F | . | . | . | . | N | . | . | . |
| OPZ03153.1 | . | . | . | . | . | . | Q | . | . |
| MBK9212880.1 | . | . | . | . | I | . | N | . | . |
| MBS1488621.1 | H | . | . | . | . | . | . | . | . |

FIRMICUTES

| MAGE-A2 | E | Y | L | Q | L | V | F | G | I |
|---|---|---|---|---|---|---|---|---|---|
| MBO6286936.1 | A | . | . | . | . | . | . | S | . |
| WP_133444878.1 | V | . | . | . | V | . | . | . | . |
| WP_048924608.1 | . | . | . | . | . | I | . | P | . |
| MBQ6625054.1 | . | . | . | . | I | . | . | N | . |

BACTEROIDETES

| MAGE-A2 | E | Y | L | Q | L | V | F | G | I |
|---|---|---|---|---|---|---|---|---|---|
| WP_071503703.1 | R | . | . | H | . | . | . | . | . |
| RYZ30368.1 | . | . | . | . | . | . | Y | . | F |
| MBQ6688332.1 | . | . | . | . | M | A | . | . | . |

FIRMICUTES

| SAGE | L | Y | A | T | V | I | H | D | I |
|---|---|---|---|---|---|---|---|---|---|
| MBO4775486.1 | Y | . | . | . | L | . | . | . | . |
| WP_115453929.1 | . | . | . | . | A | . | . | E | . |
| MBO4863242.1 | . | . | . | S | . | L | . | . | . |
| MBD5156035.1 | . | . | . | . | . | F | Q | . | . |
| WP_091272910.1 | . | . | L | . | I | . | . | . | . |

BACTEROIDETES

| SAGE | L | Y | A | T | V | I | H | D | I |
|---|---|---|---|---|---|---|---|---|---|
| WP_204530868.1 | . | . | S | . | . | . | . | E | . |

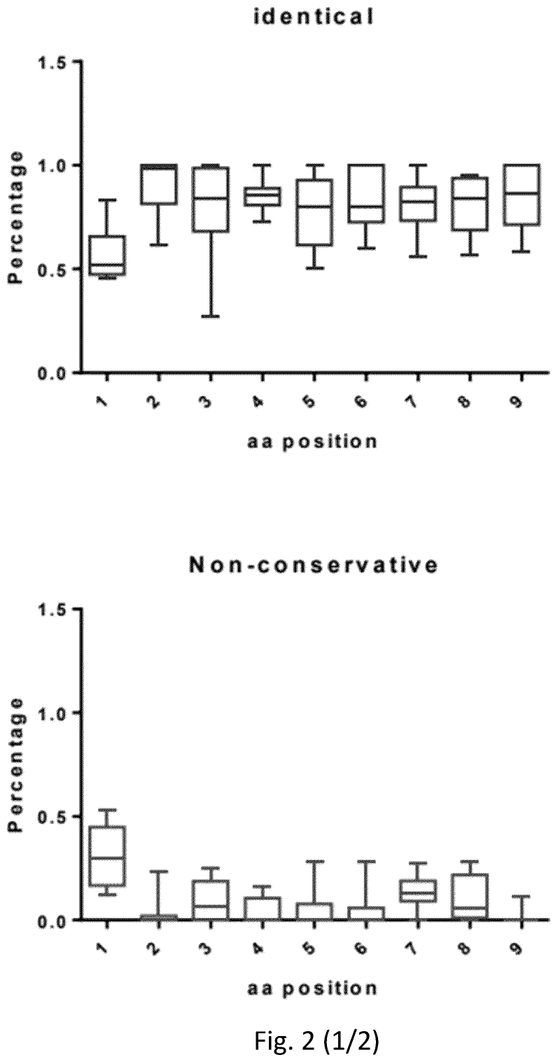

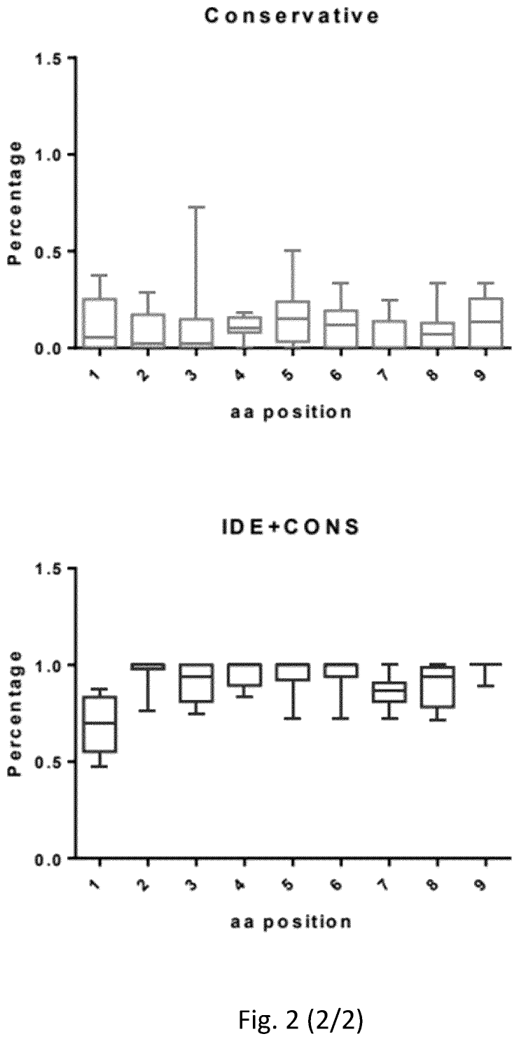

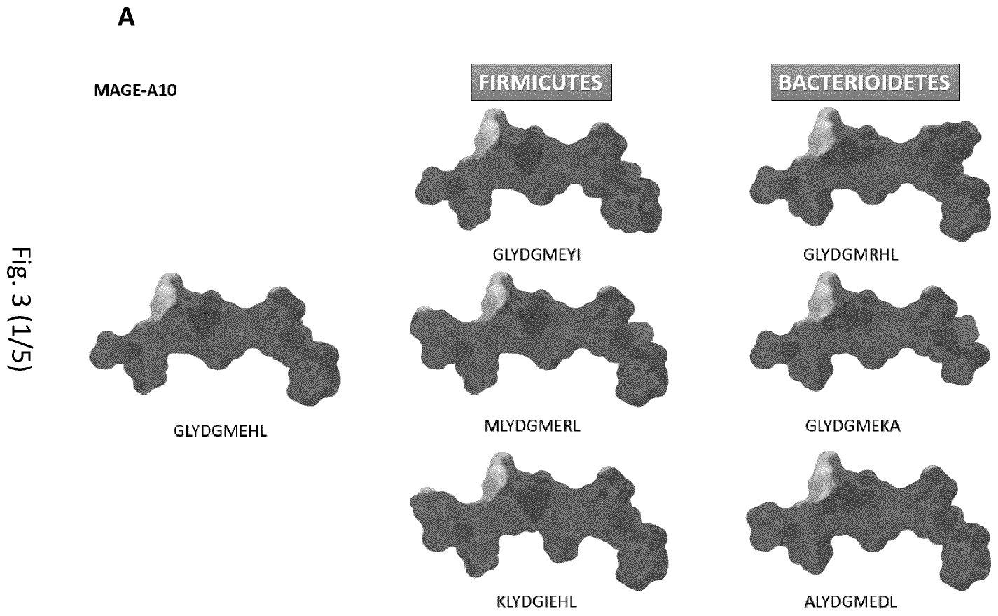

Indeed, referring to the sequences reported in Table 3, the average percentage of identical residues at each position is 82.8%, ranging from 79.7% at position 3 to 89.5% at position 2. Summing up also the conservative substitutions, the average percentage of identical residues at each position is 93.6%, ranging from 86.7% at position 7 to 98.3% at position 9. Consequently, the average percentage of non-conservative substitutions at each position is only 6.39%, ranging from 1.6% at position 9 to 13.2% at position 7. The only exception is represented by the position 1 which appears to be more permissive to non-conservative substitutions which, on average, are the 30.9% ranging from 12.5% (MAGE-C2) to 52.6% (KM-HN-1) (FIG. 2).

Strikingly, bacterial peptides identical to TAAs were identified. Indeed, considering the HLA*02:01 restricted peptides, the VLPDVFIRV peptide (SEQ ID No 4) derived from the ACT domain-containing protein [Oscillospiraceae bacterium] is identical to the GnTV TAA; the KVVEFLAML peptide (SEQ ID No 6) derived from the helix-turn-helix transcriptional regulator [Clostridia bacterium] is identical to the MAGE-C1 TAA. Similarly, considering the HLA*24:02 restricted peptides, the EYLSLSDKI peptide (SEQ ID No 10) derived from the SMI1/KNR4 family protein Roseburia sp. 1XD42-69 is identical to the KM-HN-1 TAA (Tables 1 and 2).

In order to verify whether predicted paired TAA and bacterial epitopes share similar contact residues with both the HLA molecule and the TCR, epitope modeling and molecular docking were performed (Ragone C et al., PMID: 34049932). This was possible only for HLA-A*0201 restricted epitopes, due to the lack of crystallized structures including both HLA and TCR for other alleles deposited in the PDB. Moreover, epitopes crystallized with the HLA-A*0201 and the TCR showing sequence homology with TAA peptides were not found. Therefore, fully aware of the possible caveats, the 1AO7 crystallized complex including the HTLV-I TAX epitope was used as general template to conduct the analyses (Garboczi, D. N., Ghosh, P., Utz, U., Fan, Q. R., Biddison, W. E., Wiley, D. C. (1996) Nature 384:134-141).

Among the TAAs analyzed in the present study, inventors focused on the MAGE-A3 and MAGE-A10 which have been identified in several cancers of different histological origin, including non-small cell lung cancers (NSCLC), bladder cancers, esophageal and head and neck cancers, and sarcomas (28). In particular, MAGE-A3 is overexpressed in multiple tumor types including melanoma (29) and lung cancer (30) and its presence has been associated with worse prognosis in colorectal cancer (31), cutaneous squamous cell carcinoma (32), undifferentiated pleomorphic sarcoma/myxofibrosarcoma (33). Consequently, structural and conformational homology of peptides derived from microbiota species with these TAAs may have a strong impact on several cancers.

### Results

### MAGE-A10 GLYDGMEHL TAA

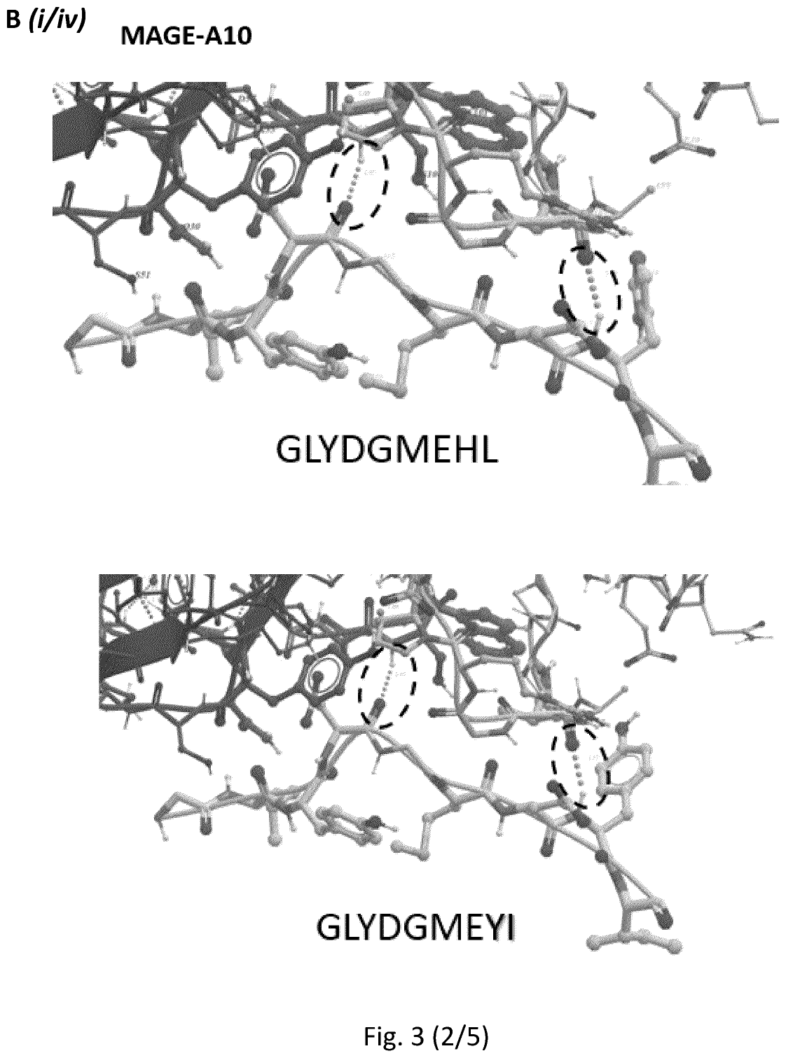

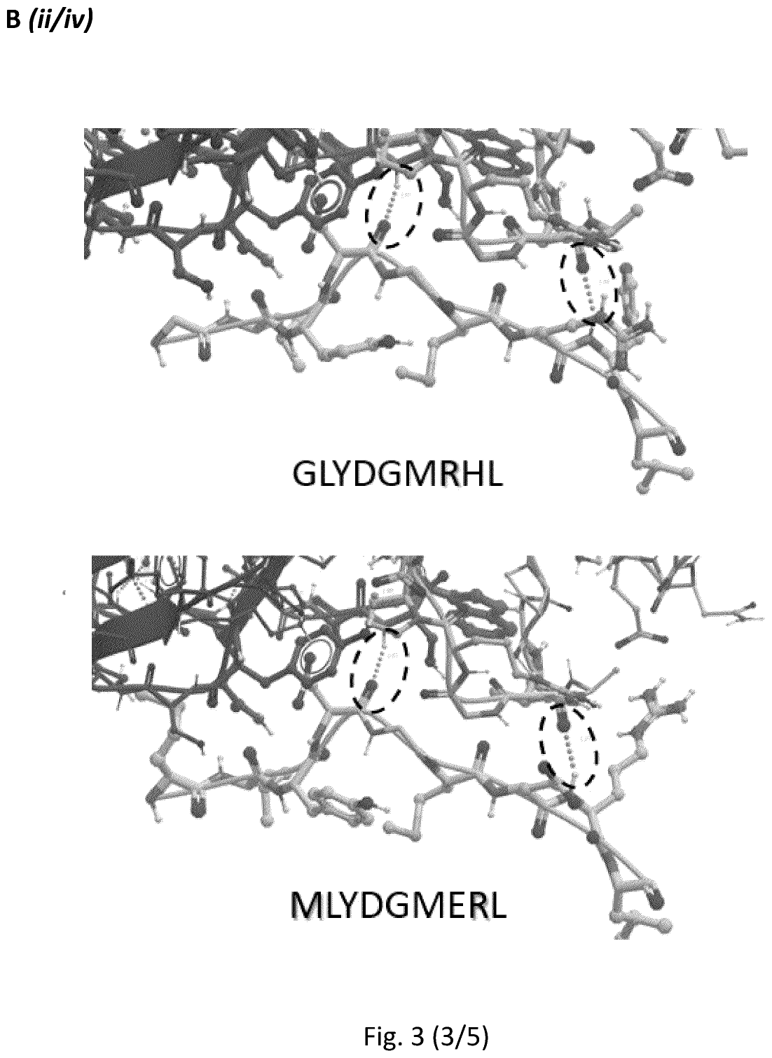

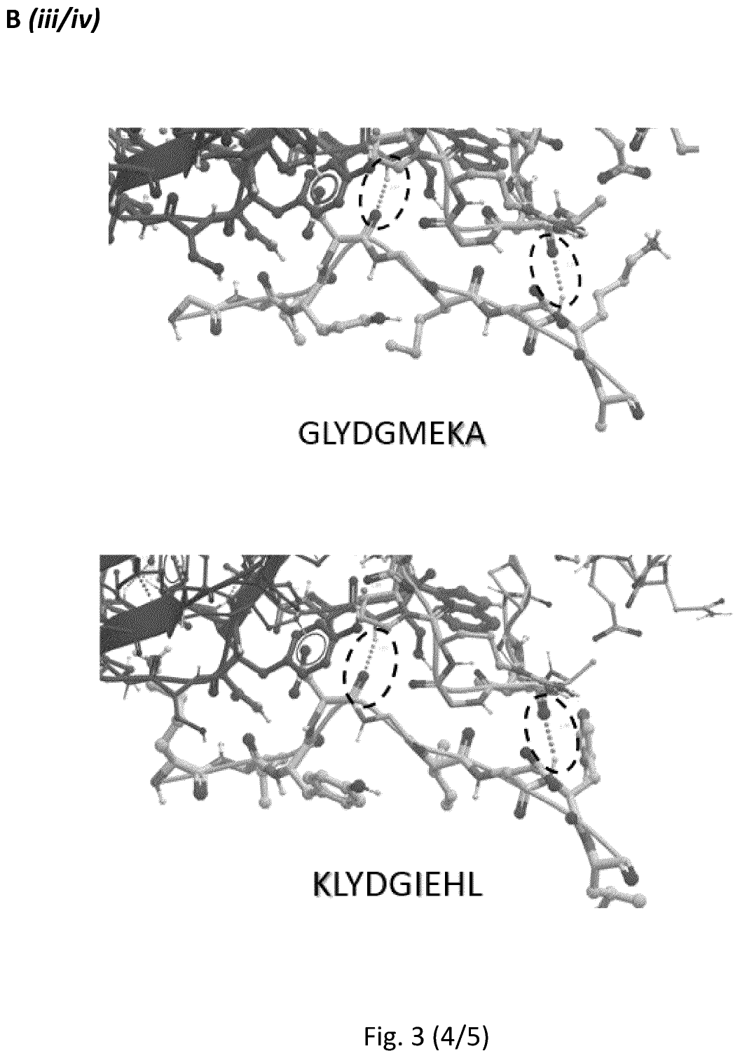

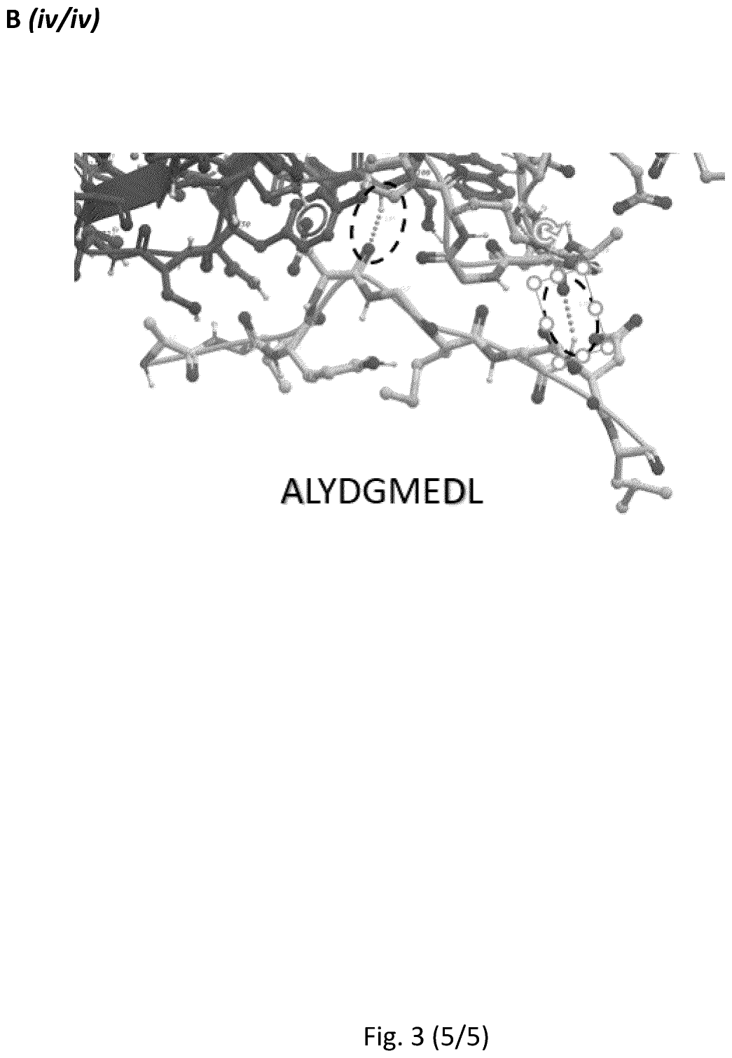

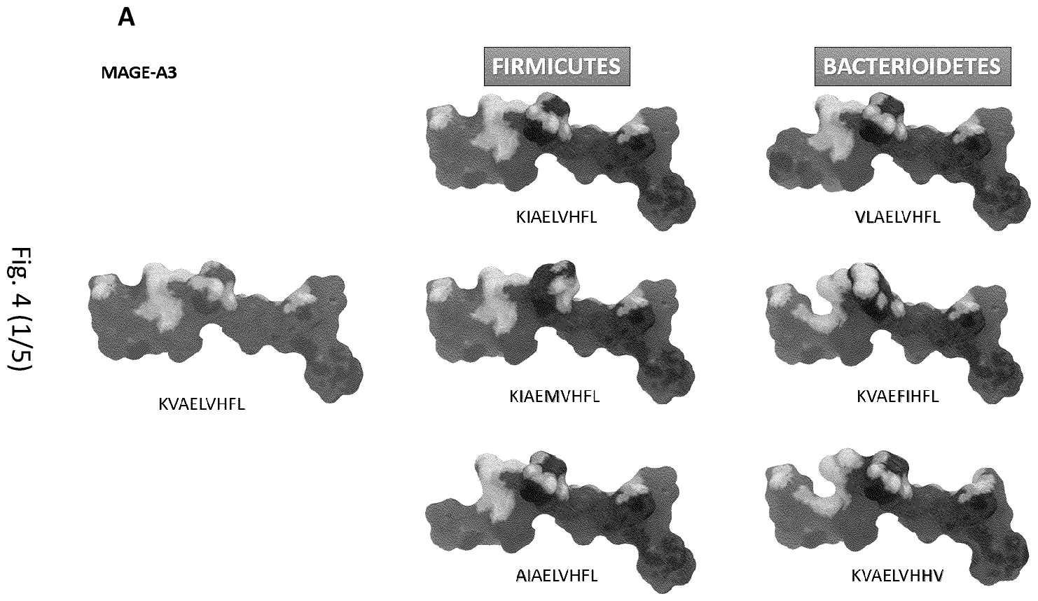

The structural and conformational analyses confirm that the MAGE-A10 GLYDGMEHL (SEQ ID No 1) peptide shows a very similar interacting pattern with the HLA and the TCR when compared to the GLYDGMEYI (SEQ ID No 13), MLYDGMERL (SEQ ID No 24) and KLYDGIEHL (SEQ ID No 23) peptides of the Firmicutes phylum, as well as the GLYDGMRHL (SEQ ID No 79), GLYDGMEKA (SEQ ID No 75) and ALYDGMEDL (SEQ ID No 76) peptides of the Bacteriodetes phylum (FIG. 3A). Furthermore, the same pattern of hydrogen bonds with identical distances is observed in the paired peptides between the residues in position 4 and 8 of the peptides and the Ser100 of the α chain and Leu98 of the β chain of TCR, respectively. This would suggest that the TCR clones targeting the TAA and microbiota peptides would share the same α and β chains (FIG. 3B).

### MAGE-A3 KVAELVHFL TAA

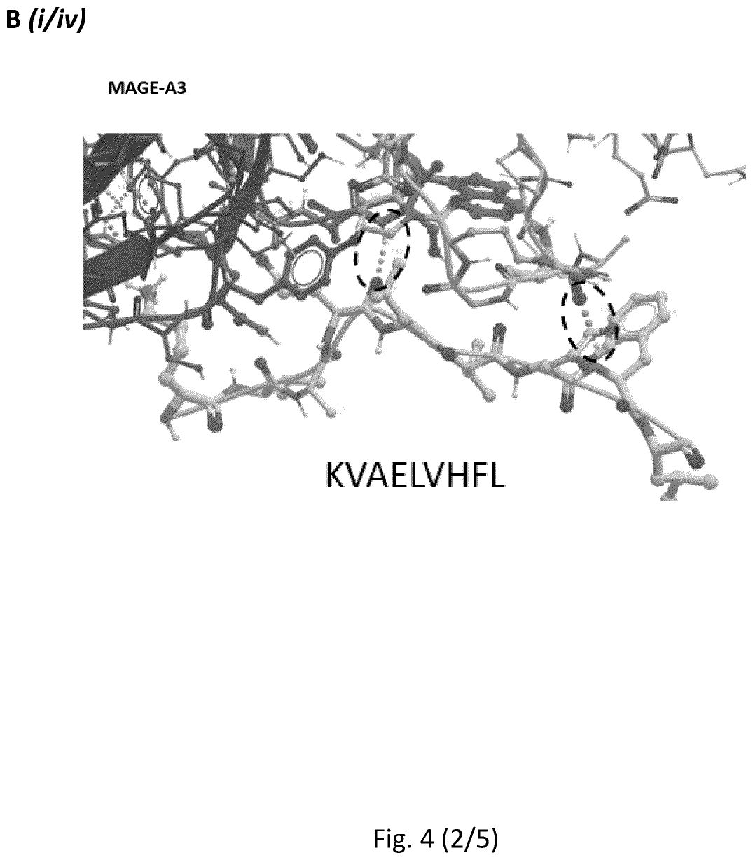

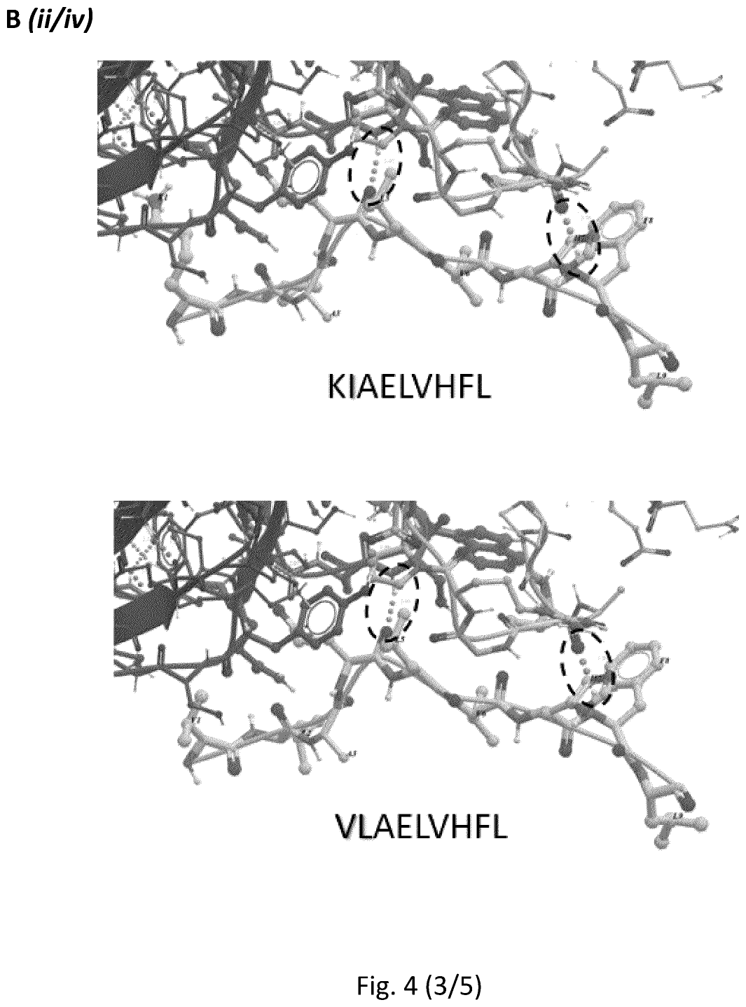

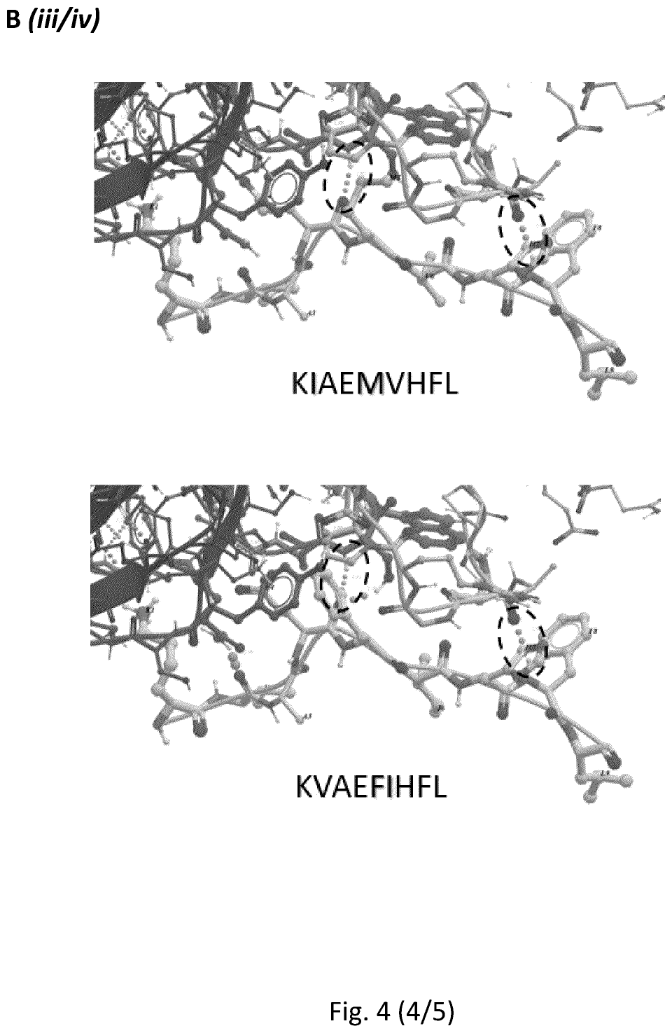

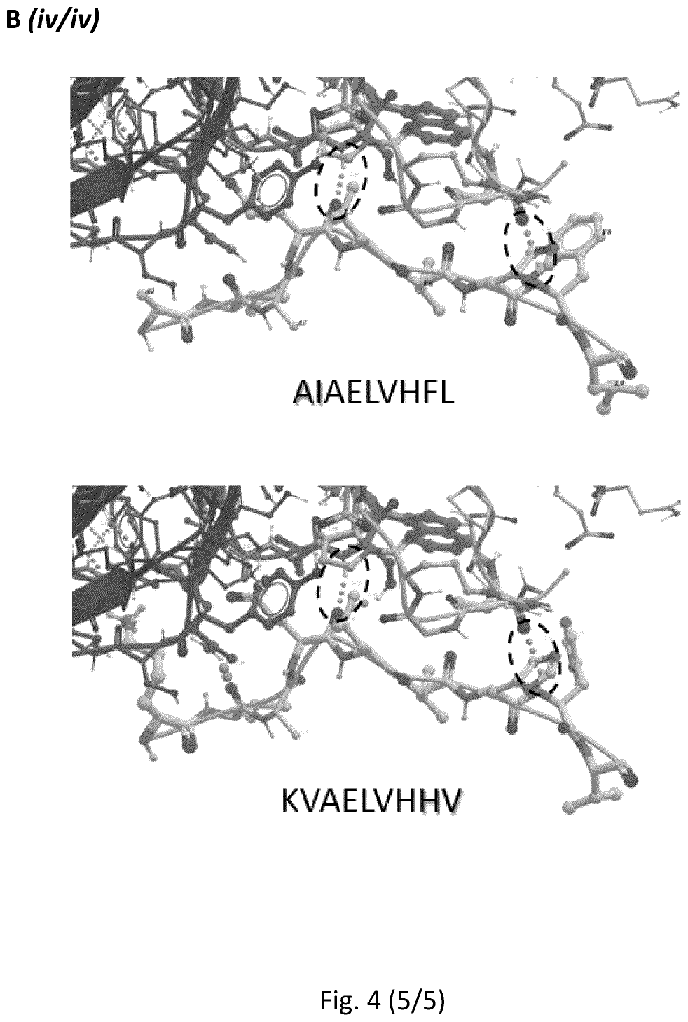

The structural and conformational analyses confirm that the MAGE-A3 KVAELVHFL (SEQ ID No 2) peptide shows a very similar interacting pattern with the HLA and the TCR when compared to the KIAELVHFL (SEQ ID No 99), KIAEMVHFL (SEQ ID No 100) and AIAELVHFL (SEQ ID No 102) peptides of the Firmicutes phylum as well as the VLAELVHFL (SEQ ID No 134) of the Bacteriodetes phylum. On the contrary, the contact patterns of the KVAEFIHFL (SEQ ID No 137) and KVAELVHHV (SEQ ID No 138) peptides is significantly different (FIG. 4A). This is reflected also in the pattern of hydrogen bonds with the α and β chains of TCR. Indeed, in addition to the usual bonds between the residues in position 4 and 8 of the peptides and the Ser100 of the α chain and Leu98 of the β chain of TCR, the latter two peptides show two additional hydrogen bonds between the Val2 and Glu4 residues of the peptides and the Gln30 and the Ser100 residues of the α chain. This would suggest that the TCR clones targeting the TAA and microbiota KVAEFIHFL (SEQ ID No 137) and KVAELVHHV (SEQ ID No 138) peptides would share only the β chain but the α chain would be different (FIG. 4B).

### MAGE-A3 FLWGPRALV TAA

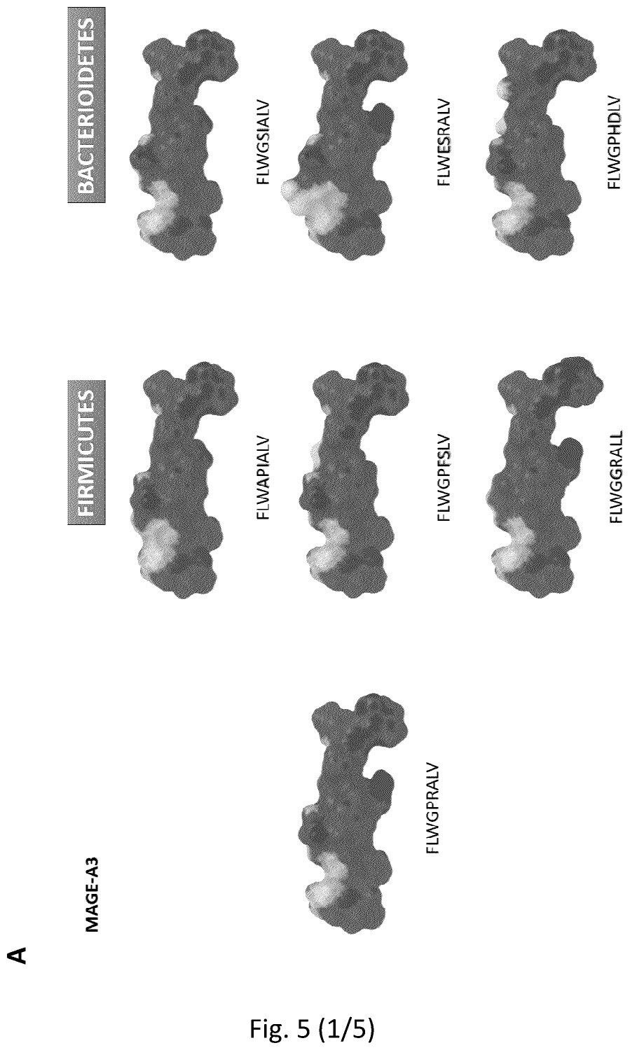

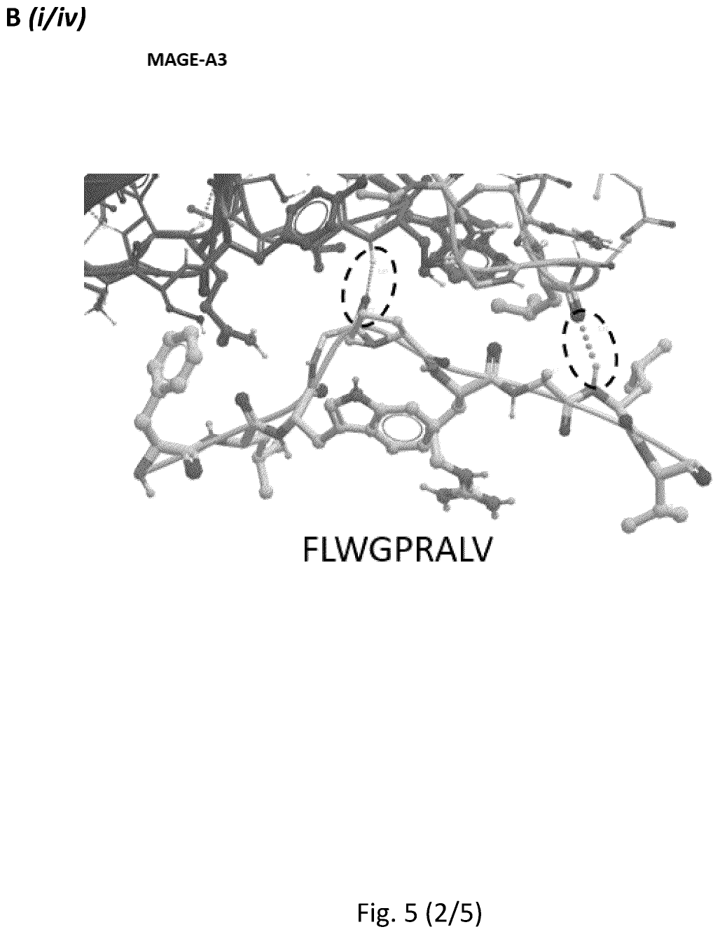

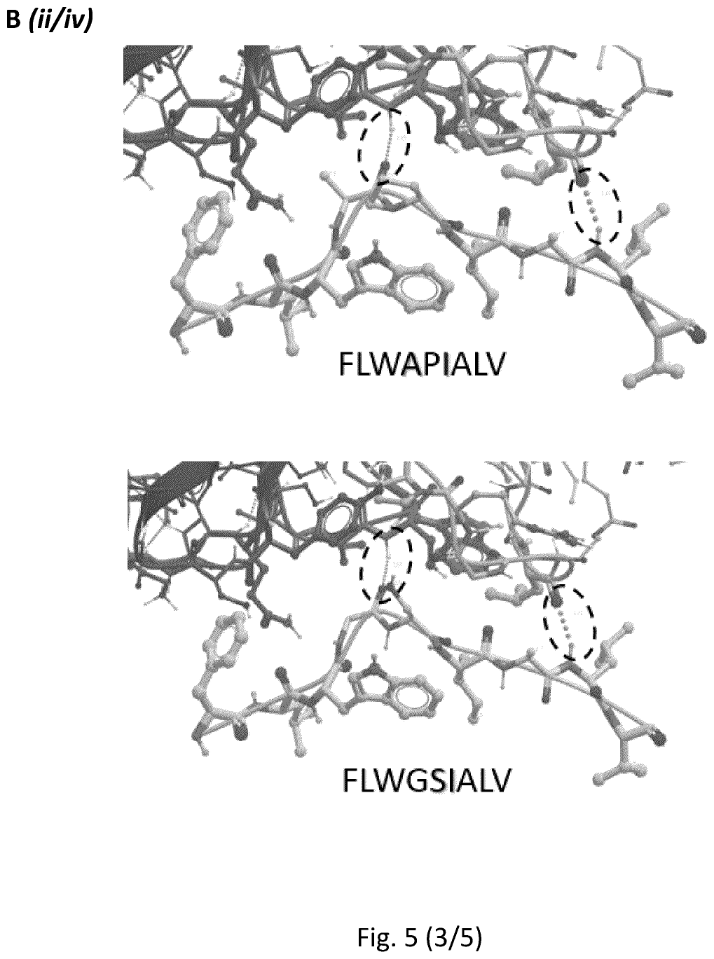

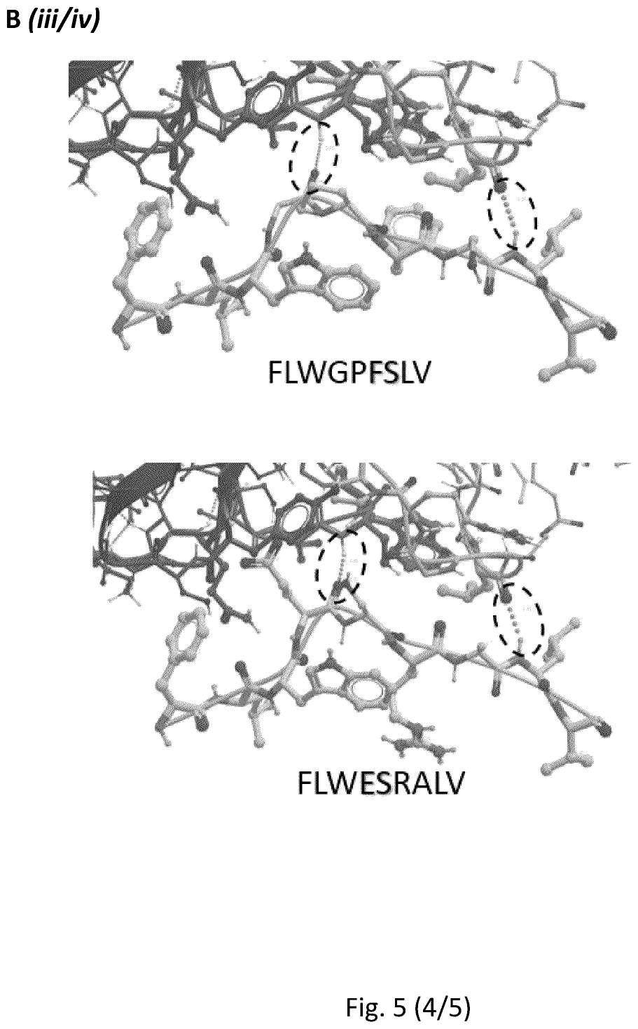

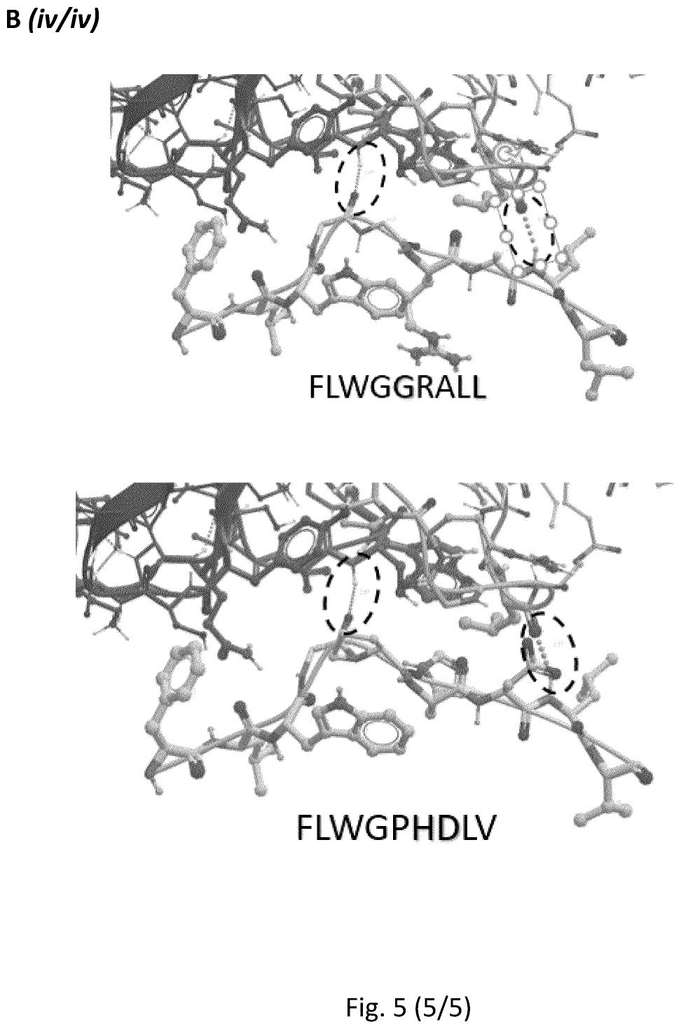

The structural and conformational analyses confirm that the MAGE-A3 FLWGPRALV (SEQ ID No 3) peptide shows the most similar interacting pattern with the HLA and the TCR when compared to the FLWGGRALL (SEQ ID No 164) peptide of the Firmicutes phylum and the FLWGSIALV (SEQ ID No 182) peptide of the Bacteriodetes phylum. On the contrary, the FLWGPFSLV (SEQ ID No 162) peptide of the Firmicutes phylum and the FLWGPHDLV (SEQ ID No 184) peptide of the Bacteriodetes phylum share only the interacting pattern with the TCR β chain; while the FLWAPIALV (SEQ ID No 160) peptide of the Firmicutes phylum and the FLWESRALV (SEQ ID No 183) peptide of the Bacteriodetes phylum share only the interacting pattern with the TCR α chain (FIG. 5A).

Regardless such a differences, an identical pattern of hydrogen bonds with the α and β chains of TCR is observed between the residues in position 4 and 8 of the peptides and the Ser100 of the α chain and Leu98 of the β chain of TCR (FIG. 5B).

The data reported in the present study show for the first time the high homology in the linear sequence as well as in structure and conformation between TAAs and peptides derived from microbiota species of the Firmicutes and the Bacteriodetes phyla, which together account for 90% of gut microbiota. Such a high level of homology, which reaches 100% in some cases, strongly suggests a cross-recognition by T cells with TCR sharing similar or identical α and β chains. In support to this hypothesis, the inventors have recently shown that CD8⁺ T cells cross-react with TAAs and peptides derived from viruses, sharing the same level of homology observed in the present study (26).

Consequently, depending on the stage of the human life when the individuals will encounter the microbiota species, such homology may represent a favorable or unfavorable factor. Indeed, if the microbiota species have colonized the gastrointestinal tract in the first few months of life, the immune system could recognize the derived peptides as self-antigens and the specific T cell clones would be removed. In this case, a tumor lesion presenting a homologous TAA would have a selective advantage for establishing and progressing with a poor prognosis.

On the contrary, if the microbiota species have colonized the gastrointestinal tract later during life, the immune system could recognize the derived peptides as non self-antigens and specific memory T cell clones would be established. In this case, as for a preventive vaccine, a tumor lesion presenting a homologous TAA would promptly recall the memory T cells able to eliminate or control tumor growth with an improved prognosis.

These findings open a new horizon in the mutual interaction between the microbiota and immune response in humans with a potential profound impact on tumor development and progression. Moreover, they provide a completely novel class of antigens to be used as anti-cancer preventive vaccination administered also as food integrators.

### REFERENCES

1. Thursby E, Juge N. Introduction to the human gut microbiota. Biochem J. 2017 May 16; 474 (11): 1823-1836. doi: 10.1042/BCJ20160510. Review. PubMed PMID: 28512250; PubMed Central PMCID: PMC5433529
2. Arumugam M, et al., Enterotypes of the human gut microbiome. Nature. 2011 May 12; 473 (7346): 174-80. doi: 10.1038/nature09944. Epub 2011 Apr. 20. PubMed PMID: 21508958; PubMed Central PMCID: PMC3728647
3. Flint H J, Scott K P, Louis P, Duncan S H. The role of the gut microbiota in nutrition and health. Nat Rev Gastroenterol Hepatol. 2012 October; 9 (10): 577-89. doi: 10.1038/nrgastro.2012.156. eCollection 2012 October. Review. PubMed PMID: 22945443
4. Arboleya S, et al., Establishment and development of intestinal microbiota in preterm neonates. FEMS Microbiol Ecol. 2012 March; 79 (3): 763-72. doi: 10.1111/j.1574-6941.2011.01261.x. Epub 2011 Dec. 15. PubMed PMID: 22126419.
5. Dominguez-Bello M G, et al., Delivery mode shapes the acquisition and structure of the initial microbiota across multiple body habitats in newborns. Proc Natl Acad Sci USA. 2010 Jun. 29; 107 (26): 11971-5. doi: 10.1073/pnas.1002601107. Epub 2010 Jun. 21. PubMed PMID: 20566857; PubMed Central PMCID: PMC2900693.
6. Fallani M, Young D, Scott J, Norin E, Amarri S, Adam R, Aguilera M, Khanna S, Gil A, Edwards C A, Doré J. Intestinal microbiota of 6-week-old infants across Europe: geographic influence beyond delivery mode, breast-feeding, and antibiotics. J Pediatr Gastroenterol Nutr. 2010 July; 51 (1): 77-84. doi: 10.1097/MPG.0b013e3181dlblle. PubMed PMID: 20479681.
7. Fallani M, Amarri S, Uusijarvi A, Adam R, Khanna S, Aguilera M, Gil A, Vieites J M, Norin E, Young D, Scott J A, Dore J, Edwards C A, The Infabio Team. Determinants of the human infant intestinal microbiota after the introduction of first complementary foods in infant samples from five European centres. Microbiology (Reading). 2011 May; 157 (Pt 5): 1385-1392. doi: 10.1099/mic.0.042143-0. Epub 2011 Feb. 17. PubMed PMID: 21330436.
8. Yatsunenko T, Rey F E, Manary M J, Trehan I, Dominguez-Bello M G, Contreras M, Magris M, Hidalgo G, Baldassano R N, Anokhin A P, Heath A C, Warner B, Reeder J, Kuczynski J, Caporaso J G, Lozupone C A, Lauber C, Clemente J C, Knights D, Knight R, Gordon J I. Human gut microbiome viewed across age and geography. Nature. 2012 May 9; 486 (7402): 222-7. doi: 10.1038/nature11053. PubMed PMID: 22699611; PubMed Central PMCID: PMC3376388.
9. Rinninella E, Raoul P, Cintoni M, Franceschi F, Miggiano GAD, Gasbarrini A, Mele M C. What is the Healthy Gut Microbiota Composition? A Changing Ecosystem across Age, Environment, Diet, and Diseases. Microorganisms. 2019 Jan. 10; 7 (1). doi: 10.3390/microorganisms7010014. Review. PubMed PMID: 30634578; PubMed Central PMCID: PMC6351938.
10. Khosravi A, Mazmanian S K. Disruption of the gut microbiome as a risk factor for microbial infections. Curr Opin Microbiol. 2013 April; 16 (2): 221-7. doi: 10.1016/j.mib.2013.03.009. Epub 2013 Apr. 15. Review. PubMed PMID: 23597788; PubMed Central PMCID: PMC5695238.
11. Brestoff J R, Artis D. Commensal bacteria at the interface of host metabolism and the immune system. Nat Immunol. 2013 July; 14 (7): 676-84. doi: 10.1038/ni.2640. Review. PubMed PMID: 23778795; PubMed Central PMCID: PMC4013146.
12. Fan Y, Pedersen O. Gut microbiota in human metabolic health and disease. Nat Rev Microbiol. 2021 January; 19 (1): 55-71. doi: 10.1038/s41579-020-0433-9. Epub 2020 Sep. 4. Review. PubMed PMID: 32887946.
13. Chen Y, Zhou J, Wang L. Role and Mechanism of Gut Microbiota in Human Disease. Front Cell Infect Microbiol. 2021; 11:625913. doi: 10.3389/fcimb.2021.625913. eCollection 2021. Review. PubMed PMID: 33816335; PubMed Central PMCID: PMC8010197.
14. Sepich-Poore G D, Zitvogel L, Straussman R, Hasty J, Wargo J A, Knight R. The microbiome and human cancer. Science. 2021 Mar. 26; 371 (6536). doi: 10.1126/science.abc4552. Review. PubMed PMID: 33766858.
15. Vivarelli S, Salemi R, Candido S, Falzone L, Santagati M, Stefani S, Torino F, Banna G L, Tonini G, Libra M. Gut Microbiota and Cancer: From Pathogenesis to Therapy. Cancers (Basel). 2019 Jan. 3; 11 (1). doi: 10.3390/cancers11010038. Review. PubMed PMID: 30609850; PubMed Central PMCID: PMC6356461.
16. Paulos C M, Wrzesinski C, Kaiser A, Hinrichs C S, Chieppa M, Cassard L, Palmer D C, Boni A, Muranski P, Yu Z, Gattinoni L, Antony P A, Rosenberg S A, Restifo N P. Microbial translocation augments the function of adoptively transferred self/tumor-specific CD8⁺ T cells via TLR4 signaling. J Clin Invest. 2007 August; 117 (8): 2197-204. doi: 10.1172/JCI32205. PubMed PMID: 17657310; PubMed Central PMCID: PMC1924500.
17. Gur C, Ibrahim Y, Isaacson B, Yamin R, Abed J, Gamliel M, Enk J, Bar-On Y, Stanietsky-Kaynan N, Coppenhagen-Glazer S, Shussman N, Almogy G, Cuapio A, Hofer E, Mevorach D, Tabib A, Ortenberg R, Markel G, Miklić K, Jonjic S, Brennan C A, Garrett W S, Bachrach G, Mandelboim O. Binding of the Fap2 protein of Fusobacterium nucleatum to human inhibitory receptor TIGIT protects tumors from immune cell attack. Immunity. 2015 Feb. 17; 42 (2): 344-355. doi: 10.1016/j.immuni.2015.01.010. Epub 2015 Feb. 10. PubMed PMID: 25680274; PubMed Central PMCID: PMC4361732.
18. Round J L, Mazmanian S K. Inducible Foxp3⁺ regulatory T-cell development by a commensal bacterium of the intestinal microbiota. Proc Natl Acad Sci USA. 2010 Jul. 6; 107 (27): 12204-9. doi: 10.1073/pnas.0909122107. Epub 2010 Jun. 21. PubMed PMID: 20566854; PubMed Central PMCID: PMC2901479.
19. Shen Y, Giardino Torchia M L, Lawson G W, Karp C L, Ashwell J D, Mazmanian S K. Outer membrane vesicles of a human commensal mediate immune regulation and disease protection. Cell Host Microbe. 2012 Oct. 18; 12 (4): 509-20. doi: 10.1016/j.chom.2012.08.004. Epub 2012 Sep. 20. PubMed PMID: 22999859; PubMed Central PMCID: PMC3895402.
20. Ivanov I I, Atarashi K, Manel N, Brodie E L, Shima T, Karaoz U, Wei D, Goldfarb K C, Santee C A, Lynch S V, Tanoue T, Imaoka A, Itoh K, Takeda K, Umesaki Y, Honda K, Littman D R. Induction of intestinal Th17 cells by segmented filamentous bacteria. Cell. 2009 Oct. 30; 139 (3): 485-98. doi: 10.1016/j.cell.2009.09.033. PubMed PMID: 19836068; PubMed Central PMCID: PMC2796826.
21. Rinninella E, Raoul P, Cintoni M, Franceschi F, Miggiano GAD, Gasbarrini A, Mele M C. What is the Healthy Gut Microbiota Composition? A Changing Ecosystem across Age, Environment, Diet, and Diseases. Microorganisms. 2019 Jan. 10; 7 (1). doi: 10.3390/microorganisms7010014. Review. PubMed PMID: 30634578; PubMed Central PMCID: PMC6351938.
22. Wooldridge L, Ekeruche-Makinde J, van den Berg H A, Skowera A, Miles J J, Tan M P, Dolton G, Clement M, Llewellyn-Lacey S, Price D A, Peakman M, Sewell A K. A single autoimmune T cell receptor recognizes more than a million different peptides. J Biol Chem. 2012 Jan. 6; 287 (2): 1168-77. doi: 10.1074/jbc.M111.289488. Epub 2011 Nov. 18. PubMed PMID: 22102287; PubMed Central PMCID: PMC3256900.
23. Sewell A K. Why must T cells be cross-reactive?. Nat Rev Immunol. 2012 September; 12 (9): 669-77. doi: 10.1038/nri3279. Review. PubMed PMID: 22918468; PubMed Central PMCID: PMC7097784.
24. Zhang L, Udaka K, Mamitsuka H, Zhu S. Toward more accurate pan-specific MHC-peptide binding prediction: a review of current methods and tools. Brief Bioinform. 2012 May; 13 (3): 350-64. doi: 10.1093/bib/bbr060. Epub 2011 Sep. 22. Review. PubMed PMID: 21949215.
25. Binkowski T A, Marino S R, Joachimiak A. Predicting HLA class I non-permissive amino acid residues substitutions. PLOS One. 2012; 7 (8): e41710. doi: 10.1371/journal.pone.0041710. Epub 2012 Aug. 8. PubMed PMID: 22905104; PubMed Central PMCID: PMC3414483.
26. Ragone C, Manolio C, Cavalluzzo B, Mauriello A, Tornesello M L, Buonaguro F M, Castiglione F, Vitagliano L, Iaccarino E, Ruvo M, Tagliamonte M, Buonaguro L. Identification and validation of viral antigens sharing sequence and structural homology with tumor-associated antigens (TAAs). J Immunother Cancer. 2021 May; 9 (5). doi: 10.1136/jitc-2021-002694. PubMed PMID: 34049932; PubMed Central PMCID: PMC8166618.
27. Buonaguro L, Cerullo V. Pathogens: Our Allies against Cancer?. Mol Ther. 2021 Jan. 6; 29 (1): 10-12. doi: 10.1016/j.ymthe.2020.12.005. Epub 2020 Dec. 5. Review. PubMed PMID: 33308440; PubMed Central PMCID: PMC7791079.
28. Juretic A, Spagnoli G C, Schultz-Thater E, Sarcevic B. Cancer/testis tumour-associated antigens: immunohistochemical detection with monoclonal antibodies. Lancet Oncol. 2003 February; 4 (2): 104-9. doi: 10.1016/s1470-2045 (03) 00982-3. Review. PubMed PMID: 12573352.
29. Gaugler B, Van den Eynde B, van der Bruggen P, Romero P, Gaforio J J, De Plaen E, Lethé B, Brasseur F, Boon T. Human gene MAGE-3 codes for an antigen recognized on a melanoma by autologous cytolytic T lymphocytes. J Exp Med. 1994 Mar. 1; 179 (3): 921-30. doi: 10.1084/jem. 179.3.921. PubMed PMID: 8113684; PubMed Central PMCID: PMC2191409.
30. Sienel W, Varwerk C, Linder A, Kaiser D, Teschner M, Delire M, Stamatis G, Passlick B. Melanoma associated antigen (MAGE)-A3 expression in Stages I and II non-small cell lung cancer: results of a multi-center study. Eur J Cardiothorac Surg. 2004 January; 25 (1): 131-4. doi: 10.1016/j.ejcts.2003.09.015. PubMed PMID: 14690745.
31. Tarnowski M, Czerewaty M, Deskur A, Safranow K, Marlicz W, Urasińska E, Ratajczak M Z, Starzyńska T. Expression of Cancer Testis Antigens in Colorectal Cancer: New Prognostic and Therapeutic Implications. Dis Markers. 2016; 2016:1987505. doi: 10.1155/2016/1987505. Epub 2016 Aug. 18. PubMed PMID: 27635108; PubMed Central PMCID: PMC5007337.
32. Abikhair M, Roudiani N, Mitsui H, Krueger J G, Pavlick A, Lee J, Therrien J P, Meehan S A, Felsen D, Carucci J A. MAGEA3 Expression in Cutaneous Squamous Cell Carcinoma Is Associated with Advanced Tumor Stage and Poor Prognosis. J Invest Dermatol. 2017 March; 137 (3): 775-778. doi: 10.1016/j.jid.2016.10.036. Epub 2016 Nov. 5. PubMed PMID: 27826009.
33. Conley A P, Wang W L, Livingston J A, Ravi V, Tsai J W, Ali A, Ingram D R, Lowery C D, Roland C L, Somaiah N, Hwu P, Yee C, Subbiah V, Futreal A, Lazar A J, Patel S, Roszik J. MAGE-A3 is a Clinically Relevant Target in Undifferentiated Pleomorphic Sarcoma/Myxofibrosarcoma. Cancers (Basel). 2019 May 15; 11 (5). doi: 10.3390/cancers 11050677. PubMed PMID: 31096717; PubMed Central PMCID: PMC6562561.

1. A composition comprising a peptide derived from the human microbiota and a pharmaceutically acceptable excipient, wherein said at least one peptide comprises a core sequence of 9 amino acids wherein said core sequence has 6, 7 or 8 amino acids identical and in the same position with respect to the tumor-associated antigenic epitope sequence GLYDGMEHL (SEQ ID No 1) and the remaining amino acids are different with respect to the amino acids in the same position of said epitope sequence.

2. The composition according to claim 1 wherein said peptide is selected from the group consisting of SEQ ID No. 13-90.

3. The composition according to claim 1 wherein said peptide is selected from the group consisting of SEQ ID No 13, 16, 21, 23, 24, 27, 28, 29, 30, 33, 36, 38, 39, 40, 41, 43, 44, 45, 46, 55, 66, 67, 72, 73, 74, 75, 76, 77, 78, 79, 82, 82, 84, 85, 86, 87 and 88, preferably is selected from the group consisting of SEQ ID No 13, 23, 24, 75, 76 and 79.

4. (canceled)

5. The composition according to claim 1 comprising a further peptide wherein said further peptide comprises a core sequence of 9 amino acids wherein said core sequence has at least 6 amino acids identical and in the same position with respect to one tumor-associated antigenic epitope sequence selected from the group consisting of:
(SEQ ID No. 2)
KVAELVHFL;
(SEQ ID No 3)
FLWGPRALV;
(SEQ ID No 4)
VLPDVFIRV;
(SEQ ID No 5)
KVLEYVIKV;
(SEQ ID No 6)
KVVEFLAML;
(SEQ ID No 7)
ALKDVEERV;
(SEQ ID No 8)
KASEKIFYV;
(SEQ ID No 9)
NYNNFYRFL;
(SEQ ID No 10)
EYLSLSDKI;
(SEQ ID No 11)
EYLQLVFGI;
and
(SEQ ID No 12)
LYATVIHDI.

6. The composition according to claim 5 wherein the further peptide is selected from the group consisting of SEQ ID No. 91-631.

7. The composition according to claim 5 wherein said further peptide is selected from the group consisting of: SEQ ID No 91-197.

8. The composition according to claim 7 wherein said peptide is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, and 196, preferably is selected from the group consisting of SEQ ID No 99, 100, 102, 134, 137, 138, 160, 162, 164, 182, 183, 184.

9. A composition comprising a peptide derived from the human microbiota and a pharmaceutically acceptable excipient, wherein said peptide comprises a core sequence of 9 amino acids wherein said core sequence has 6, 7 or 8 amino acids identical and in the same position with respect to one tumor-associated antigenic epitope sequence selected from the group consisting of:
(SEQ ID No. 2)
KVAELVHFL;
(SEQ ID No 3)
FLWGPRALV;
(SEQ ID No 4)
VLPDVFIRV;
(SEQ ID No 5)
KVLEYVIKV;
(SEQ ID No 6)
KVVEFLAML;
(SEQ ID No 7)
ALKDVEERV;
(SEQ ID No 8)
KASEKIFYV;
(SEQ ID No 9)
NYNNFYRFL;
(SEQ ID No 10)
EYLSLSDKI;
(SEQ ID No 11)
EYLQLVFGI;
and
(SEQ ID No 12)
LYATVIHDI.
and remaining amino acids are different with respect to the amino acids in the same position of said tumor-associated antigenic epitope sequence.

10. The composition according to claim 9 wherein said peptide peptide is selected from the group consisting of SEQ ID No. 91-209, SEQ ID No. 211-327, SEQ ID No. 329-507, and SEQ ID No. 509-631.

11. The composition according to claim 10 wherein said further peptide is selected from the group consisting of: SEQ ID No 91-197.

12. The composition according to claim 11 wherein said peptide is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, or and 196, preferably is selected from the group consisting of SEQ ID No 99, 100, 102, 134, 137, 138, 160, 162, 164, 182, 183, 184.

13. A method for the treatment and/or prevention of cancer, wherein the cancer is characterized by increased expression of an antigen selected from the group consisting of: MAGE-A10, MAGE-A3, GnTV, LRPAP1, MAGE-C1, MAGE-C2, SSX-2, KM-HN-1, KM-HN-2, MAGE-A2, and SAGE, comprising administering a composition of claim 1 to a subject in need thereof.

14. The method according to claim 13 wherein the cancer is a solid tumor, preferably the solid tumor is selected from melanoma, lung cancer bladder cancers, esophageal and head and neck cancers, and sarcomas, colorectal cancer, gastric cancer, cutaneous squamous cell carcinoma, undifferentiated pleomorphic sarcoma/myxofibrosarcoma, non-small cell lung cancer, diffuse large B-cell lymphoma, hepatocellular carcinoma, breast cancer, pancreatic cancer, gynecology cancer.

15. The method according to claim 13 further comprising administering a further anticancer therapeutic agent.

16. A vaccine or immunogenic composition comprising the composition of claim 1 and a pharmaceutically acceptable vehicle or excipient.

17. (canceled)

18. A dietary supplement or replacement comprising a peptide derived from the human microbiota, wherein said peptide comprises a core sequence of 9 amino acids wherein said core sequence has 6, 7 or 8 amino acids identical and in the same position with respect to the tumor-associated antigenic epitope sequence GLYDGMEHL (SEQ ID No 1) and the remaining amino acids are different with respect to the amino acids in the same position of said epitope sequence.

19. The dietary supplement or replacement according to claim 18 wherein said peptide is selected from the group consisting of SEQ ID No. 13-90.

20. The dietary supplement or replacement according to claim claim 18 wherein said peptide is selected from the group consisting of SEQ ID No 13, 16, 21, 23, 24, 27, 28, 29, 30, 33, 36, 38, 39, 40, 41, 43, 44, 45, 46, 55, 66, 67, 72, 73, 74, 75, 76, 77, 78, 79, 82, 82, 84, 85, 86, 87 and 88, preferably is selected from the group consisting of SEQ ID No 13, 23, 24, 75, 76 and 79.

21. The dietary supplement or replacement according to claim 18 comprising at least one further peptide derived from the human microbiota, wherein said at least one further peptide comprises a core sequence of 9 amino acids wherein said core sequence has at least 6 amino acids identical and in the same position with respect to one tumor-associated antigenic epitope sequence selected from the group consisting of:
(SEQ ID No. 2)
KVAELVHFL;
(SEQ ID No 3)
FLWGPRALV;
(SEQ ID No 4)
VLPDVFIRV;
(SEQ ID No 5)
KVLEYVIKV;
(SEQ ID No 6)
KVVEFLAML;
(SEQ ID No 7)
ALKDVEERV;
(SEQ ID No 8)
KASEKIFYV;
(SEQ ID No 9)
NYNNFYRFL;
(SEQ ID No 10)
EYLSLSDKI;
(SEQ ID No 11)
EYLQLVFGI;
and
(SEQ ID No 12)
LYATVIHDI.
and a pharmaceutically acceptable vehicle or excipient.

22. The dietary supplement or replacement according to claim 21 wherein the further peptide is selected from the group consisting of SEQ ID No. 91-631.

23. The dietary supplement or replacement according to claim 22 wherein said further peptide is selected from the group consisting of: SEQ ID No 91-197.

24. The dietary supplement or replacement according to claim 23 wherein said peptide is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, and 196, preferably is selected from the group consisting of SEQ ID No 99, 100, 102, 134, 137, 138, 160, 162, 164, 182, 183, 184.

25. A dietary supplement or replacement comprising a peptide derived from the human microbiota, wherein said peptide comprises a core sequence of 9 amino acids wherein said core sequence has 6, 7 or 8 amino acids identical and in the same position with respect to one tumor-associated antigenic epitope sequence selected from the group consisting of:
(SEQ ID No. 2)
KVAELVHFL;
(SEQ ID No 3)
FLWGPRALV;
(SEQ ID No 4)
VLPDVFIRV;
(SEQ ID No 5)
KVLEYVIKV;
(SEQ ID No 6)
KVVEFLAML;
(SEQ ID No 7)
ALKDVEERV;
(SEQ ID No 8)
KASEKIFYV;
(SEQ ID No 9)
NYNNFYRFL;
(SEQ ID No 10)
EYLSLSDKI;
(SEQ ID No 11)
EYLQLVFGI;
and
(SEQ ID No 12)
LYATVIHDI.
and remaining amino acids are different with respect to the amino acids in the same position of said tumor-associated antigenic epitope sequence.

26. The dietary supplement or replacement according to claim 25 wherein said peptide is selected from the group consisting of SEQ ID No. 91-209, SEQ ID No. 211-327, SEQ ID No. 329-507, and SEQ ID No. 509-631.

27. The dietary supplement or replacement according to claim 26 wherein said further peptide is selected from the group consisting of: SEQ ID No 91-197, preferably said at least one peptide is selected from the group consisting of: SEQ ID No 134, 136, 137, 138, 142, 144, 146, 147, 150, 152, 153, 154, 155, 156, 160, 162, 163, 164, 166, 167, 169, 170, 274, 276, 177, 178, 282, 183, 184, 185, 186, 187, 188, 189, 190, 191, 194, 195, and 196, preferably is selected from the group consisting of SEQ ID No 99, 100, 102, 134, 137, 138, 160, 162, 164, 182, 183, 184.

28. (canceled)

29. (canceled)

30. (canceled)


SEQUENCE LISTING (631 sequences)

SEQ ID NO: 1 - protein, 9 aa, synthetic construct
  REGION 1..9: note = peptide
GLYDGMEHL

SEQ ID NO: 2 - protein, 9 aa, synthetic construct
  REGION 1..9: note = peptide
KVAELVHFL

SEQ ID NO: 3 - protein, 9 aa, synthetic construct
  REGION 1..9: note = peptide
FLWGPRALV

SEQ ID NO: 4 - protein, 9 aa, synthetic construct
  REGION 1..9: note = peptide
VLPDVFIRV

SEQ ID NO: 5 - protein, 9 aa, synthetic construct
  REGION 1..9: note = peptide
KVLEYVIKV

SEQ ID NO: 6 - protein, 9 aa, synthetic construct
  REGION 1..9: note = peptide
KVVEFLAML

SEQ ID NO: 7 - protein, 9 aa, synthetic construct
  REGION 1..9: note = peptide
ALKDVEERV

SEQ ID NO: 8 - protein, 9 aa, synthetic construct
  REGION 1..9: note = peptide
KASEKIFYV

SEQ ID NO: 9 - protein, 9 aa, synthetic construct
  REGION 1..9: note = peptide
NYNNFYRFL

SEQ ID NO: 10 - protein, 9 aa, synthetic construct
  REGION 1..9: note = peptide
EYLSLSDKI

SEQ ID NO: 11 - protein, 9 aa, synthetic construct
  REGION 1..9: note = peptide
EYLQLVFGI

SEQ ID NO: 12 - protein, 9 aa, synthetic construct
  REGION 1..9: note = peptide
LYATVIHDI

SEQ ID NO: 13 - protein, 9 aa, synthetic construct
  REGION 1..9: note = peptide
GLYDGMEYI

SEQ ID NO: 14 - protein, 9 aa, synthetic construct
  REGION 1..9: note = peptide
GLYDSMEYV

SEQ ID NO: 15 - protein, 9 aa, synthetic construct
  REGION 1..9: note = peptide
GLYDGMLGV

SEQ ID NO: 16 - protein, 9 aa, synthetic construct
  REGION 1..9: note = peptide
YLYDDMEHL

SEQ ID NO: 17 - protein, 9 aa, synthetic construct
  REGION 1..9: note = peptide
ALYDGMEEV

SEQ ID NO: 18 - protein, 9 aa, synthetic construct
  REGION 1..9: note = peptide
GLYDGMNTV

SEQ ID NO: 19 - protein, 9 aa, synthetic construct
  REGION 1..9: note = peptide
YLYNGIEHI

SEQ ID NO: 20 - protein, 9 aa, synthetic construct
  REGION 1..9: note = peptide
VMYDGMEYL

SEQ ID NO: 21 - protein, 9 aa, synthetic construct
  REGION 1..9: note = peptide
KLYDGLESL

SEQ ID NO: 22 - protein, 9 aa, synthetic construct
  REGION 1..9: note = peptide
KLYDGIEHL

SEQ ID NO: 23 - protein, 9 aa, synthetic construct
  REGION 1..9: note = peptide
GLYDGVEHV

SEQ ID NO: 24 - protein, 9 aa, synthetic construct
  REGION 1..9: note = peptide
MLYDGMERL

SEQ ID NO: 25 - protein, 9 aa, synthetic construct
  REGION 1..9: note = peptide
MLYDGMERL

SEQ ID NO: 26 - protein, 9 aa, synthetic construct
  REGION 1..9: note = peptide
SLYDGLEQL

SEQ ID NO: 27 - protein, 9 aa, synthetic construct
  REGION 1..9: note = peptide
RLYDGLEHL

SEQ ID NO: 28 - protein, 9 aa, synthetic construct
  REGION 1..9: note = peptide
GLYEGMNHL

SEQ ID NO: 29 - protein, 9 aa, synthetic construct
  REGION 1..9: note = peptide
ALYDGMEQL

SEQ ID NO: 30 - protein, 8 aa, synthetic construct
  REGION 1..8: note = peptide
GLFDSMEH

SEQ ID NO: 31 - protein, 9 aa, synthetic construct
  REGION 1..9: note = peptide
GMYDMMEHL

SEQ ID NO: 32 - protein, 9 aa, synthetic construct
  REGION 1..9: note = peptide
GIYDGMEYI

SEQ ID NO: 33 - protein, 9 aa, synthetic construct
  REGION 1..9: note = peptide
QLYNGMEHV

SEQ ID NO: 34 - protein, 9 aa, synthetic construct
  REGION 1..9: note = peptide
QLYNGMEHV

SEQ ID NO: 35 - protein, 9 aa, synthetic construct
  REGION 1..9: note = peptide
GLYEAMEEA

SEQ ID NO: 36 - protein, 9 aa, synthetic construct
  REGION 1..9: note = peptide
GLYDVMEHI

SEQ ID NO: 37 - protein, 9 aa, synthetic construct
  REGION 1..9: note = peptide
GLFDGMENA

SEQ ID NO: 38 - protein, 9 aa, synthetic construct
  REGION 1..9: note = peptide
LLYAGMEHL

SEQ ID NO: 39 - protein, 9 aa, synthetic construct
  REGION 1..9: note = peptide
KLYDGMEDL

SEQ ID NO: 40 - protein, 9 aa, synthetic construct
  REGION 1..9: note = peptide
GLYETMEHL

SEQ ID NO: 41 - protein, 9 aa, synthetic construct
  REGION 1..9: note = peptide
ALYDTMEHL

SEQ ID NO: 42 - protein, 9 aa, synthetic construct
  REGION 1..9: note = peptide
GLYDGMNRA

SEQ ID NO: 43 - protein, 9 aa, synthetic construct
  REGION 1..9: note = peptide
GLYSGMEHI

SEQ ID NO: 44 - protein, 9 aa, synthetic construct
  REGION 1..9: note = peptide
GLYDGMEFT

SEQ ID NO: 45 - protein, 9 aa, synthetic construct
  REGION 1..9: note = peptide
NLYDNMEHL

SEQ ID NO: 46 - protein, 9 aa, synthetic construct
  REGION 1..9: note = peptide
GLYEGMKHL

SEQ ID NO: 47 - protein, 9 aa, synthetic construct
  REGION 1..9: note = peptide
TLYDGMNDL

SEQ ID NO: 48 - protein, 9 aa, synthetic construct
  REGION 1..9: note = peptide
VLNDGMEHL

SEQ ID NO: 49 - protein, 9 aa, synthetic construct
  REGION 1..9: note = peptide
GLYDGMVFS

SEQ ID NO: 50 - protein, 9 aa, synthetic construct
  REGION 1..9: note = peptide
GIYDGMETA

SEQ ID NO: 51 - protein, 9 aa, synthetic construct
  REGION 1..9: note = peptide
TLYDGMQQC

SEQ ID NO: 52 - protein, 9 aa, synthetic construct
  REGION 1..9: note = peptide
KLYDGMEVC

SEQ ID NO: 53 - protein, 9 aa, synthetic construct
  REGION 1..9: note = peptide
NIYDGLEHL

SEQ ID NO: 54 - protein, 9 aa, synthetic construct
  REGION 1..9: note = peptide
GVFEGMEHL

SEQ ID NO: 55 - protein, 9 aa, synthetic construct
  REGION 1..9: note = peptide
GLYDGMEVT

SEQ ID NO: 56 - protein, 9 aa, synthetic construct
  REGION 1..9: note = peptide
VIYDAMEHL

SEQ ID NO: 57 - protein, 9 aa, synthetic construct
  REGION 1..9: note = peptide
VIYDAMEHL

SEQ ID NO: 58 - protein, 9 aa, synthetic construct
  REGION 1..9: note = peptide
GIYDGMEKI

SEQ ID NO: 59 - protein, 9 aa, synthetic construct
  REGION 1..9: note = peptide
HLTDGMEHL

SEQ ID NO: 60 - protein, 9 aa, synthetic construct
  REGION 1..9: note = peptide
PLYDGMETL

SEQ ID NO: 61 - protein, 9 aa, synthetic construct
  REGION 1..9: note = peptide
SMKDGMEHL

SEQ ID NO: 62 - protein, 9 aa, synthetic construct
  REGION 1..9: note = peptide
TLYDGMRQT

SEQ ID NO: 63 - protein, 9 aa, synthetic construct
  REGION 1..9: note = peptide
YLYDGLDHI

SEQ ID NO: 64 - protein, 9 aa, synthetic construct
  REGION 1..9: note = peptide
FLYDSVAHL

SEQ ID NO: 65 - protein, 9 aa, synthetic construct
  REGION 1..9: note = peptide
VLYDGIEHV

SEQ ID NO: 66 - protein, 9 aa, synthetic construct
  REGION 1..9: note = peptide
GMYDGLEHL

SEQ ID NO: 67 - protein, 9 aa, synthetic construct
  REGION 1..9: note = peptide
GLYDGLENL

SEQ ID NO: 68 - protein, 9 aa, synthetic construct
  REGION 1..9: note = peptide
TLADGMEYL

SEQ ID NO: 69 - protein, 9 aa, synthetic construct
  REGION 1..9: note = peptide
YLYNGLKHL

SEQ ID NO: 70 - protein, 9 aa, synthetic construct
  REGION 1..9: note = peptide
RLYDGMDEL

SEQ ID NO: 71 - protein, 9 aa, synthetic construct
  REGION 1..9: note = peptide
RLYDGMDAL

SEQ ID NO: 72 - protein, 9 aa, synthetic construct
  REGION 1..9: note = peptide
GLYDGMELA

SEQ ID NO: 73 - protein, 9 aa, synthetic construct
  REGION 1..9: note = peptide
VLYDNMEHL

SEQ ID NO: 74 - protein, 9 aa, synthetic construct
  REGION 1..9: note = peptide
GLYDEMEQL

SEQ ID NO: 75 - protein, 9 aa, synthetic construct
  REGION 1..9: note = peptide
ALYDGMEDL

SEQ ID NO: 76 - protein, 9 aa, synthetic construct
  REGION 1..9: note = peptide
GLYDGMEKA

SEQ ID NO: 77 - protein, 9 aa, synthetic construct
  REGION 1..9: note = peptide
GLFDGMKHL

SEQ ID NO: 78 - protein, 9 aa, synthetic construct
  REGION 1..9: note = peptide
GLYDGMRHL

SEQ ID NO: 79 - protein, 9 aa, synthetic construct
  REGION 1..9: note = peptide
ALYNGMDHL

SEQ ID NO: 80 - protein, 9 aa, synthetic construct
  REGION 1..9: note = peptide
GLYHGMNLL

SEQ ID NO: 81 - protein, 9 aa, synthetic construct
  REGION 1..9: note = peptide
FVYDGTEHA

SEQ ID NO: 82 - protein, 9 aa, synthetic construct
  REGION 1..9: note = peptide
ELYDGLEHL

SEQ ID NO: 83 - protein, 9 aa, synthetic construct
  REGION 1..9: note = peptide
YLYDGQEQF

SEQ ID NO: 84 - protein, 9 aa, synthetic construct
  REGION 1..9: note = peptide
AIYDGMEHA

SEQ ID NO: 85 - protein, 9 aa, synthetic construct
  REGION 1..9: note = peptide
LLYDPKEHL

SEQ ID NO: 86 - protein, 9 aa, synthetic construct
  REGION 1..9: note = peptide
VTYDGMEHV

SEQ ID NO: 87 - protein, 9 aa, synthetic construct
  REGION 1..9: note = peptide
FVYDGLEHT

SEQ ID NO: 88 - protein, 9 aa, synthetic construct
  REGION 1..9: note = peptide
GLYDGMEKT

SEQ ID NO: 89 - protein, 9 aa, synthetic construct
  REGION 1..9: note = peptide
KTYDGLNHL

SEQ ID NO: 90 - protein, 9 aa, synthetic construct
  REGION 1..9: note = peptide
RTYEEMEQL

SEQ ID NO: 91 - protein, 9 aa, synthetic construct
  REGION 1..9: note = peptide
YLTELLHFL

SEQ ID NO: 92 - protein, 9 aa, synthetic construct
  REGION 1..9: note = peptide
KLAEMVHFL

SEQ ID NO: 93 - protein, 9 aa, synthetic construct
  REGION 1..9: note = peptide
ILAELVNFV

SEQ ID NO: 94 - protein, 9 aa, synthetic construct
  REGION 1..9: note = peptide
FVSELVHFI

SEQ ID NO: 95 - protein, 9 aa, synthetic construct
  REGION 1..9: note = peptide
FQAELVNYL

SEQ ID NO: 96 - protein, 9 aa, synthetic construct
  REGION 1..9: note = peptide
VLHELVHFL

SEQ ID NO: 97 - protein, 9 aa, synthetic construct
  REGION 1..9: note = peptide
VLHELVHFL

SEQ ID NO: 98 - protein, 9 aa, synthetic construct
  REGION 1..9: note = peptide
RLAELVDFL

SEQ ID NO: 99 - protein, 9 aa, synthetic construct
  REGION 1..9: note = peptide
KIAELVHFL

SEQ ID NO: 100 - protein, 9 aa, synthetic construct
  REGION 1..9: note = peptide
KIAEMVHFL

SEQ ID NO: 101 - protein, 9 aa, synthetic construct
  REGION 1..9: note = peptide
KLAELVRFL

SEQ ID NO: 102 - protein, 9 aa, synthetic construct
  REGION 1..9: note = peptide
AIAELVHFL

SEQ ID NO: 103 - protein, 9 aa, synthetic construct
  REGION 1..9: note = peptide
KVAELVLYL

SEQ ID NO: 104 - protein, 9 aa, synthetic construct
  REGION 1..9: note = peptide
MVAELVQYL

SEQ ID NO: 105 - protein, 9 aa, synthetic construct
  REGION 1..9: note = peptide
RVAELIHFL

SEQ ID NO: 106 - protein, 9 aa, synthetic construct
  REGION 1..9: note = peptide
KVAEFINFL

SEQ ID NO: 107 - protein, 9 aa, synthetic construct
  REGION 1..9: note = peptide
YAAELIHFL

SEQ ID NO: 108 - protein, 9 aa, synthetic construct
  REGION 1..9: note = peptide
KITELVHFL

SEQ ID NO: 109 - protein, 9 aa, synthetic construct
  REGION 1..9: note = peptide
HIAELVHFL

SEQ ID NO: 110 - protein, 9 aa, synthetic construct
  REGION 1..9: note = peptide
KVSDLLHFL

SEQ ID NO: 111 - protein, 9 aa, synthetic construct
  REGION 1..9: note = peptide
NVAEIVHFL

SEQ ID NO: 112 - protein, 9 aa, synthetic construct
  REGION 1..9: note = peptide
TVADLVHYL

SEQ ID NO: 113 - protein, 9 aa, synthetic construct
  REGION 1..9: note = peptide
SVAEAVHFL

SEQ ID NO: 114 - protein, 9 aa, synthetic construct
  REGION 1..9: note = peptide
KVAGLVLFL

SEQ ID NO: 115 - protein, 9 aa, synthetic construct
  REGION 1..9: note = peptide
QVAELVQFM

SEQ ID NO: 116 - protein, 9 aa, synthetic construct
  REGION 1..9: note = peptide
KVVETVHFL

SEQ ID NO: 117 - protein, 9 aa, synthetic construct
  REGION 1..9: note = peptide
  SITE 6: note = misc_feature - Xaa can be any naturally occurring amino acid
YAAELXHFL

SEQ ID NO: 118 - protein, 9 aa, synthetic construct
  REGION 1..9: note = peptide
MAAELVHFL

SEQ ID NO: 119 - protein, 9 aa, synthetic construct
  REGION 1..9: note = peptide
RQSALVHFL

SEQ ID NO: 120 - protein, 9 aa, synthetic construct
  REGION 1..9: note = peptide
MVAELVHIL

SEQ ID NO: 121 - protein, 9 aa, synthetic construct
  REGION 1..9: note = peptide
ATAEMLHFL

SEQ ID NO: 122 - protein, 9 aa, synthetic construct
  REGION 1..9: note = peptide
QVAELVHFL

SEQ ID NO: 123 - protein, 9 aa, synthetic construct
  REGION 1..9: note = peptide
ELAELVHAL

SEQ ID NO: 124 - protein, 9 aa, synthetic construct
  REGION 1..9: note = peptide
SVAELVDFL

SEQ ID NO: 125 - protein, 9 aa, synthetic construct
  REGION 1..9: note = peptide
AVTELVHRV

SEQ ID NO: 126 - protein, 9 aa, synthetic construct
  REGION 1..9: note = peptide
KVTDLVQFL

SEQ ID NO: 127 - protein, 9 aa, synthetic construct
  REGION 1..9: note = peptide
IVAESVHFA

SEQ ID NO: 128 - protein, 9 aa, synthetic construct
  REGION 1..9: note = peptide
KVAELVNML

SEQ ID NO: 129 - protein, 9 aa, synthetic construct
  REGION 1..9: note = peptide
KVAELVDHL

SEQ ID NO: 130 - protein, 9 aa, synthetic construct
  REGION 1..9: note = peptide
KVAELVHVA

SEQ ID NO: 131 - protein, 9 aa, synthetic construct
  REGION 1..9: note = peptide
KVAELQHTA

SEQ ID NO: 132 - protein, 9 aa, synthetic construct
  REGION 1..9: note = peptide
KVAELQHTA

SEQ ID NO: 133 - protein, 9 aa, synthetic construct
  REGION 1..9: note = peptide
LLAENIHFL

SEQ ID NO: 134 - protein, 9 aa, synthetic construct
  REGION 1..9: note = peptide
VLAELVHFL

SEQ ID NO: 135 - protein, 9 aa, synthetic construct
  REGION 1..9: note = peptide
KVAEFVALV

SEQ ID NO: 136 - protein, 9 aa, synthetic construct
  REGION 1..9: note = peptide
KVAEFIHFL

SEQ ID NO: 137 - protein, 9 aa, synthetic construct
  REGION 1..9: note = peptide
KVAEFIHFL

SEQ ID NO: 138 - protein, 9 aa, synthetic construct
  REGION 1..9: note = peptide
KVAELVHHV

SEQ ID NO: 139 - protein, 9 aa, synthetic construct
  REGION 1..9: note = peptide
YIAELVEFA

SEQ ID NO: 140 - protein, 9 aa, synthetic construct
  REGION 1..9: note = peptide
KVADLEHFV

SEQ ID NO: 141 - protein, 9 aa, synthetic construct
  REGION 1..9: note = peptide
KVADLEHFV

SEQ ID NO: 142 - protein, 9 aa, synthetic construct
  REGION 1..9: note = peptide
KVTELVHFL

SEQ ID NO: 143 - protein, 9 aa, synthetic construct
  REGION 1..9: note = peptide
LVAEILQFL

SEQ ID NO: 144 - protein, 9 aa, synthetic construct
  REGION 1..9: note = peptide
KVAALVHFL

SEQ ID NO: 145 - protein, 9 aa, synthetic construct
  REGION 1..9: note = peptide
NVAELVPYL

SEQ ID NO: 146 - protein, 9 aa, synthetic construct
  REGION 1..9: note = peptide
KVAELVSFL

SEQ ID NO: 147 - protein, 9 aa, synthetic construct
  REGION 1..9: note = peptide
KVAELVNFL

SEQ ID NO: 148 - protein, 9 aa, synthetic construct
  REGION 1..9: note = peptide
QIAELTHFL

SEQ ID NO: 149 - protein, 9 aa, synthetic construct
  REGION 1..9: note = peptide
SVAELIEFL

SEQ ID NO: 150 - protein, 9 aa, synthetic construct
  REGION 1..9: note = peptide
QVAELIHFL

SEQ ID NO: 151 - protein, 9 aa, synthetic construct
  REGION 1..9: note = peptide
GVASVIHFL

SEQ ID NO: 152 - protein, 9 aa, synthetic construct
  REGION 1..9: note = peptide
ATAELVHFL

SEQ ID NO: 153 - protein, 9 aa, synthetic construct
  REGION 1..9: note = peptide
KVADLVHFA

SEQ ID NO: 154 - protein, 9 aa, synthetic construct
  REGION 1..9: note = peptide
KVADLVHFA

SEQ ID NO: 155 - protein, 9 aa, synthetic construct
  REGION 1..9: note = peptide
KVSELVNFL

SEQ ID NO: 156 - protein, 9 aa, synthetic construct
  REGION 1..9: note = peptide
KVSELVNFL

SEQ ID NO: 157 - protein, 9 aa, synthetic construct
  REGION 1..9: note = peptide
SVSELINFL

SEQ ID NO: 158 - protein, 9 aa, synthetic construct
  REGION 1..9: note = peptide
KIVELIKFL

SEQ ID NO: 159 - protein, 9 aa, synthetic construct
  REGION 1..9: note = peptide
FLWGPVMLV

SEQ ID NO: 160 - protein, 9 aa, synthetic construct
  REGION 1..9: note = peptide
FLWAPIALV

SEQ ID NO: 161 - protein, 9 aa, synthetic construct
  REGION 1..9: note = peptide
FLWGAPVLI

SEQ ID NO: 162 - protein, 9 aa, synthetic construct
  REGION 1..9: note = peptide
FLWGPFSLV

SEQ ID NO: 163 - protein, 9 aa, synthetic construct
  REGION 1..9: note = peptide
YLWGLRALV

SEQ ID NO: 164 - protein, 9 aa, synthetic construct
  REGION 1..9: note = peptide
FLWGGRALL

SEQ ID NO: 165 - protein, 9 aa, synthetic construct
  REGION 1..9: note = peptide
FLWDPRSGV

SEQ ID NO: 166 - protein, 9 aa, synthetic construct
  REGION 1..9: note = peptide
FLWGPFALI

SEQ ID NO: 167 - protein, 9 aa, synthetic construct
  REGION 1..9: note = peptide
FLWGPAALI

SEQ ID NO: 168 - protein, 9 aa, synthetic construct
  REGION 1..9: note = peptide
YLWGKRALL

SEQ ID NO: 169 - protein, 9 aa, synthetic construct
  REGION 1..9: note = peptide
FLWNPRALL

SEQ ID NO: 170 - protein, 9 aa, synthetic construct
  REGION 1..9: note = peptide
SLWGHRALV

SEQ ID NO: 171 - protein, 9 aa, synthetic construct
  REGION 1..9: note = peptide
TLWDSRALV

SEQ ID NO: 172 - protein, 9 aa, synthetic construct
  REGION 1..9: note = peptide
TLWDSRALV

SEQ ID NO: 173 - protein, 9 aa, synthetic construct
  REGION 1..9: note = peptide
FLWGPTVDA

SEQ ID NO: 174 - protein, 9 aa, synthetic construct
  REGION 1..9: note = peptide
FLWGSRGLV

SEQ ID NO: 175 - protein, 9 aa, synthetic construct
  REGION 1..9: note = peptide
FLWGKRAQA

SEQ ID NO: 176 - protein, 9 aa, synthetic construct
  REGION 1..9: note = peptide
FLWAPRAKI

SEQ ID NO: 177 - protein, 9 aa, synthetic construct
  REGION 1..9: note = peptide
FLWGPRAKI

SEQ ID NO: 178 - protein, 9 aa, synthetic construct
  REGION 1..9: note = peptide
FLWGPRQLA

SEQ ID NO: 179 - protein, 9 aa, synthetic construct
  REGION 1..9: note = peptide
FLHGPKALV

SEQ ID NO: 180 - protein, 9 aa, synthetic construct
  REGION 1..9: note = peptide
SLWGPRSAV

SEQ ID NO: 181 - protein, 9 aa, synthetic construct
  REGION 1..9: note = peptide
ILFGPRALL

SEQ ID NO: 182 - protein, 9 aa, synthetic construct
  REGION 1..9: note = peptide
FLWGSIALV

SEQ ID NO: 183 - protein, 9 aa, synthetic construct
  REGION 1..9: note = peptide
FLWESRALV

SEQ ID NO: 184 - protein, 9 aa, synthetic construct
  REGION 1..9: note = peptide
FLWGPHDLV

SEQ ID NO: 185 - protein, 9 aa, synthetic construct
  REGION 1..9: note = peptide
FLWNKRALV

SEQ ID NO: 186 - protein, 9 aa, synthetic construct
  REGION 1..9: note = peptide
FLWNKRALV

SEQ ID NO: 187 - protein, 9 aa, synthetic construct
  REGION 1..9: note = peptide
FLWGPIGLV

SEQ ID NO: 188 - protein, 9 aa, synthetic construct
  REGION 1..9: note = peptide
RLWGPIALV

SEQ ID NO: 189 - protein, 9 aa, synthetic construct
  REGION 1..9: note = peptide
FMWGPRAEV

SEQ ID NO: 190 - protein, 9 aa, synthetic construct
  REGION 1..9: note = peptide
FLWGPKALV

SEQ ID NO: 191 - protein, 9 aa, synthetic construct
  REGION 1..9: note = peptide
FLWGLRALI

SEQ ID NO: 192 - protein, 9 aa, synthetic construct
  REGION 1..9: note = peptide
FLYGVGALL

SEQ ID NO: 193 - protein, 9 aa, synthetic construct
  REGION 1..9: note = peptide
TLWGPAPLV

SEQ ID NO: 194 - protein, 9 aa, synthetic construct
  REGION 1..9: note = peptide
FLWGRRAVV

SEQ ID NO: 195 - protein, 9 aa, synthetic construct
  REGION 1..9: note = peptide
LLWGPKALV

SEQ ID NO: 196 - protein, 9 aa, synthetic construct
  REGION 1..9: note = peptide
QLWGARALV

SEQ ID NO: 197 - protein, 9 aa, synthetic construct
  REGION 1..9: note = peptide
AMWSPRAQV

SEQ ID NO: 198 - protein, 9 aa, synthetic construct
  REGION 1..9: note = peptide
YLPDIFIRV

SEQ ID NO: 199 - protein, 9 aa, synthetic construct
  REGION 1..9: note = peptide
LLPDVLFRV

SEQ ID NO: 200 - protein, 9 aa, synthetic construct
  REGION 1..9: note = peptide
LLPDVLFRV

SEQ ID NO: 201 - protein, 9 aa, synthetic construct
  REGION 1..9: note = peptide
FLQEIFIRV

SEQ ID NO: 202 - protein, 9 aa, synthetic construct
  REGION 1..9: note = peptide
RLPDIFIRV

SEQ ID NO: 203 - protein, 9 aa, synthetic construct
  REGION 1..9: note = peptide
VLPDMFISV

SEQ ID NO: 204 - protein, 9 aa, synthetic construct
  REGION 1..9: note = peptide
KLVDVLIRI

SEQ ID NO: 205 - protein, 9 aa, synthetic construct
  REGION 1..9: note = peptide
KLVDVLIRI

SEQ ID NO: 206 - protein, 9 aa, synthetic construct
  REGION 1..9: note = peptide
KLPDVFVSI

SEQ ID NO: 207 - protein, 9 aa, synthetic construct
  REGION 1..9: note = peptide
KLPDVFVSI

SEQ ID NO: 208 - protein, 9 aa, synthetic construct
  REGION 1..9: note = peptide
ALPEIFIRV

SEQ ID NO: 209 - protein, 9 aa, synthetic construct
ALPEIFIRV

SEQ ID NO: 210 - protein, 9 aa, synthetic construct
VLPDVFIRV

SEQ ID NO: 211 - protein, 9 aa, synthetic construct
VLPDIFIKV

SEQ ID NO: 212 - protein, 9 aa, synthetic construct
VLPDVFLRV

SEQ ID NO: 213 - protein, 9 aa, synthetic construct
VLPEVFVRV

SEQ ID NO: 214 - protein, 9 aa, synthetic construct
SLPEVFSRV

SEQ ID NO: 215 - protein, 9 aa, synthetic construct
SLPEVFSRV

SEQ ID NO: 216 - protein, 9 aa, synthetic construct
VLPEIFIKV

SEQ ID NO: 217 - protein, 9 aa, synthetic construct
VLPEVFIKV

SEQ ID NO: 218 - protein, 9 aa, synthetic construct
VLPEVFLKV

SEQ ID NO: 219 - protein, 9 aa, synthetic construct
VLPEVFLKV

SEQ ID NO: 220 - protein, 9 aa, synthetic construct
VLPEVFLKV

SEQ ID NO: 221 - protein, 9 aa, synthetic construct
VLPDVFEKV

SEQ ID NO: 222 - protein, 9 aa, synthetic construct
VLPDVFEKV

SEQ ID NO: 223 - protein, 9 aa, synthetic construct
ILPDVFIRL

SEQ ID NO: 224 - protein, 9 aa, synthetic construct
ELPDVFIRC

SEQ ID NO: 225 - protein, 9 aa, synthetic construct
ELPDVFIRC

SEQ ID NO: 226 - protein, 9 aa, synthetic construct
GLPEVYIRV

SEQ ID NO: 227 - protein, 9 aa, synthetic construct
VLDDVFIRI

SEQ ID NO: 228 - protein, 9 aa, synthetic construct
ALTDVFIRI

SEQ ID NO: 229 - protein, 9 aa, synthetic construct
LLPDVFTRI

SEQ ID NO: 230 - protein, 9 aa, synthetic construct
ILPDVFRKV

SEQ ID NO: 231 - protein, 9 aa, synthetic construct
ILPDVFRKV

SEQ ID NO: 232 - protein, 9 aa, synthetic construct
ILPDVFRKV

SEQ ID NO: 233 - protein, 9 aa, synthetic construct
ILPDVFRKV

SEQ ID NO: 234 - protein, 9 aa, synthetic construct
ALPEVFRRV

SEQ ID NO: 235 - protein, 9 aa, synthetic construct
ALPEVFRRV

SEQ ID NO: 236 - protein, 9 aa, synthetic construct
VLPDTFIRL

SEQ ID NO: 237 - protein, 9 aa, synthetic construct
VLPDVFIGA

SEQ ID NO: 238 - protein, 9 aa, synthetic construct
VLPEVFRKV

SEQ ID NO: 239 - protein, 9 aa, synthetic construct
VLPEVFRKV

SEQ ID NO: 240 - protein, 9 aa, synthetic construct
NLPDVFIRI

SEQ ID NO: 241 - protein, 9 aa, synthetic construct
QLPDVFVRI

SEQ ID NO: 242 - protein, 9 aa, synthetic construct
SLPDVFICA

SEQ ID NO: 243 - protein, 9 aa, synthetic construct
FLPDVFIRC

SEQ ID NO: 244 - protein, 9 aa, synthetic construct
ELTDIFIRV

SEQ ID NO: 245 - protein, 9 aa, synthetic construct
ELTDIFIRV

SEQ ID NO: 246 - protein, 9 aa, synthetic construct
IMPDVFVPV

SEQ ID NO: 247 - protein, 9 aa, synthetic construct
LLSDVFIRV

SEQ ID NO: 248 - protein, 9 aa, synthetic construct
FLPDIFIRI

SEQ ID NO: 249 - protein, 9 aa, synthetic construct
FLPDVFIRI

SEQ ID NO: 250 - protein, 9 aa, synthetic construct
ILQDVFIRV

SEQ ID NO: 251 - protein, 9 aa, synthetic construct
ILQEVFIRV

SEQ ID NO: 252 - protein, 9 aa, synthetic construct
ILPDVFVPI

SEQ ID NO: 253 - protein, 9 aa, synthetic construct
GLPDVFLRV

SEQ ID NO: 254 - protein, 9 aa, synthetic construct
GLPDVFLRV

SEQ ID NO: 255 - protein, 9 aa, synthetic construct
GLPDVFLRV

SEQ ID NO: 256 - protein, 9 aa, synthetic construct
VMPDAFIGV

SEQ ID NO: 257 - protein, 9 aa, synthetic construct
VLQEVFIRV

SEQ ID NO: 258 - protein, 9 aa, synthetic construct
VLPDTFIRV

SEQ ID NO: 259 - protein, 9 aa, synthetic construct
IMPDIFIPL

SEQ ID NO: 260 - protein, 9 aa, synthetic construct
IMPDVFVPL

SEQ ID NO: 261 - protein, 9 aa, synthetic construct
VLPDVFIGL

SEQ ID NO: 262 - protein, 9 aa, synthetic construct
VLPDVFIAL

SEQ ID NO: 263 - protein, 9 aa, synthetic construct
VLQDVFVKL

SEQ ID NO: 264 - protein, 9 aa, synthetic construct
IIPDAIIRV

SEQ ID NO: 265 - protein, 9 aa, synthetic construct
ILPEVFARM

SEQ ID NO: 266 - protein, 9 aa, synthetic construct
VLPDVFVRA

SEQ ID NO: 267 - protein, 9 aa, synthetic construct
VLPDAFIRA

SEQ ID NO: 268 - protein, 9 aa, synthetic construct
VLPDIGIKI

SEQ ID NO: 269 - protein, 9 aa, synthetic construct
KAPDVFIRV

SEQ ID NO: 270 - protein, 9 aa, synthetic construct
VLHDVFVKT

SEQ ID NO: 271 - protein, 9 aa, synthetic construct
ELPEVAIRV

SEQ ID NO: 272 - protein, 9 aa, synthetic construct
VLRDIFINM

SEQ ID NO: 273 - protein, 9 aa, synthetic construct
RLLDYLIKV

SEQ ID NO: 274 - protein, 9 aa, synthetic construct
KVMEYLISI

SEQ ID NO: 275 - protein, 9 aa, synthetic construct
RLLEYIVKI

SEQ ID NO: 276 - protein, 9 aa, synthetic construct
KVLEYVIRV

SEQ ID NO: 277 - protein, 9 aa, synthetic construct
KVLEYVAKV

SEQ ID NO: 278 - protein, 9 aa, synthetic construct
KVLEYVIDV

SEQ ID NO: 279 - protein, 9 aa, synthetic construct
KVLEYAIMV

SEQ ID NO: 280 - protein, 9 aa, synthetic construct
KVLEYVTKV

SEQ ID NO: 281 - protein, 9 aa, synthetic construct
KVLEYAIRV

SEQ ID NO: 282 - protein, 9 aa, synthetic construct
KVLEYAIKV

SEQ ID NO: 283 - protein, 9 aa, synthetic construct
MVLDYMIKI

SEQ ID NO: 284 - protein, 9 aa, synthetic construct
KVLEYIIKI

SEQ ID NO: 285 - protein, 9 aa, synthetic construct
LVLEYLEKV

SEQ ID NO: 286 - protein, 9 aa, synthetic construct
TILEFAIKV

SEQ ID NO: 287 - protein, 9 aa, synthetic construct
KTIEYVNKV

SEQ ID NO: 288 - protein, 9 aa, synthetic construct
NVLGYVIKV

SEQ ID NO: 289 - protein, 9 aa, synthetic construct
KVLEYIIRA

SEQ ID NO: 290 - protein, 9 aa, synthetic construct
SVLEYGIKV

SEQ ID NO: 291 - protein, 9 aa, synthetic construct
KVLEYVDKI

SEQ ID NO: 292 - protein, 9 aa, synthetic construct
KVLETVRKV

SEQ ID NO: 293 - protein, 9 aa, synthetic construct
KVLEYIVKT

SEQ ID NO: 294 - protein, 9 aa, synthetic construct
KALEYVRNV

SEQ ID NO: 295 - protein, 9 aa, synthetic construct
KVKEYVEKV

SEQ ID NO: 296 - protein, 9 aa, synthetic construct
EILENVIKV

SEQ ID NO: 297 - protein, 9 aa, synthetic construct
ALLEYVIKV

SEQ ID NO: 298 - protein, 9 aa, synthetic construct
HLLEYVIKV

SEQ ID NO: 299 - protein, 9 aa, synthetic construct
KVLEYLPKV

SEQ ID NO: 300 - protein, 9 aa, synthetic construct
RILEYVIKV

SEQ ID NO: 301 - protein, 9 aa, synthetic construct
KVLEYVAKV

SEQ ID NO: 302 - protein, 9 aa, synthetic construct
SMLEYVKKV

SEQ ID NO: 303 - protein, 9 aa, synthetic construct
AVLEYVVKV

SEQ ID NO: 304 - protein, 9 aa, synthetic construct
KVIEYLIEA

SEQ ID NO: 305 - protein, 9 aa, synthetic construct
KVLEYVEKV

SEQ ID NO: 306 - protein, 9 aa, synthetic construct
KVLEYCIKV

SEQ ID NO: 307 - protein, 9 aa, synthetic construct
ALLEYLIKT

SEQ ID NO: 308 - protein, 9 aa, synthetic construct
KVLEYVQGI

SEQ ID NO: 309 - protein, 9 aa, synthetic construct
KVLNNVITV

SEQ ID NO: 310 - protein, 9 aa, synthetic construct
NVLEYVVKV

SEQ ID NO: 311 - protein, 9 aa, synthetic construct
MSLEYVLKV

SEQ ID NO: 312 - protein, 9 aa, synthetic construct
IVGEYVVKV

SEQ ID NO: 313 - protein, 9 aa, synthetic construct
KVLEYIHKA

SEQ ID NO: 314 - protein, 9 aa, synthetic construct
LVLEYVIKL

SEQ ID NO: 315 - protein, 9 aa, synthetic construct
KVLEYVKKV

SEQ ID NO: 316 - protein, 9 aa, synthetic construct
ATAEYVTKV

SEQ ID NO: 317 - protein, 9 aa, synthetic construct
KVLEYNEEL

SEQ ID NO: 318 - protein, 9 aa, synthetic construct
YFLEYVEKV

SEQ ID NO: 319 - protein, 9 aa, synthetic construct
KVLQYVKRV

SEQ ID NO: 320 - protein, 9 aa, synthetic construct
RLVEFLAML

SEQ ID NO: 321 - protein, 9 aa, synthetic construct
KILEFLAML

SEQ ID NO: 322 - protein, 9 aa, synthetic construct
KLVAFLAML

SEQ ID NO: 323 - protein, 9 aa, synthetic construct
KTVEFLAMV

SEQ ID NO: 324 - protein, 9 aa, synthetic construct
ALFTFLAML

SEQ ID NO: 325 - protein, 9 aa, synthetic construct
KVADFLAML

SEQ ID NO: 326 - protein, 9 aa, synthetic construct
KVIEFLSML

SEQ ID NO: 327 - protein, 9 aa, synthetic construct
KIVDFLAQA

SEQ ID NO: 328 - protein, 9 aa, synthetic construct
KVVEFLAML

SEQ ID NO: 329 - protein, 9 aa, synthetic construct
GVIEFLAML

SEQ ID NO: 330 - protein, 9 aa, synthetic construct
KIVEFISML

SEQ ID NO: 331 - protein, 9 aa, synthetic construct
AVVEILAML

SEQ ID NO: 332 - protein, 9 aa, synthetic construct
KVAEFVALV

SEQ ID NO: 333 - protein, 9 aa, synthetic construct
KVIEFLQDV

SEQ ID NO: 334 - protein, 9 aa, synthetic construct
KVIEFLALL

SEQ ID NO: 335 - protein, 9 aa, synthetic construct
CLNEFLPML

SEQ ID NO: 336 - protein, 9 aa, synthetic construct
FVVDFLAQL

SEQ ID NO: 337 - protein, 9 aa, synthetic construct
KVVDFLALL

SEQ ID NO: 338 - protein, 9 aa, synthetic construct
RIVDFLALL

SEQ ID NO: 339 - protein, 9 aa, synthetic construct
NLVEFIEML

SEQ ID NO: 340 - protein, 9 aa, synthetic construct
RIVDFLAQL

SEQ ID NO: 341 - protein, 9 aa, synthetic construct
IVLEFLAML

SEQ ID NO: 342 - protein, 9 aa, synthetic construct
TVLEFLAML

SEQ ID NO: 343 - protein, 9 aa, synthetic construct
SVAEFLEML

SEQ ID NO: 344 - protein, 9 aa, synthetic construct
QVAEFLAML

SEQ ID NO: 345 - protein, 9 aa, synthetic construct
ELNEFLAML

SEQ ID NO: 346 - protein, 9 aa, synthetic construct
YLKEVEEPV

SEQ ID NO: 347 - protein, 9 aa, synthetic construct
YLKDVEYRV

SEQ ID NO: 348 - protein, 9 aa, synthetic construct
YLKDVEYRV

SEQ ID NO: 349 - protein, 9 aa, synthetic construct
LLSDVEERV

SEQ ID NO: 350 - protein, 9 aa, synthetic construct
ALSDVEEGV

SEQ ID NO: 351 - protein, 9 aa, synthetic construct
YLKDIDERV

SEQ ID NO: 352 - protein, 9 aa, synthetic construct
YLKDIDERV

SEQ ID NO: 353 - protein, 9 aa, synthetic construct
ALSDVEERV

SEQ ID NO: 354 - protein, 9 aa, synthetic construct
ALNDVEERL

SEQ ID NO: 355 - protein, 9 aa, synthetic construct
MLKDIEERV

SEQ ID NO: 356 - protein, 9 aa, synthetic construct
ALKDVEEPV

SEQ ID NO: 357 - protein, 9 aa, synthetic construct
ALKDVEEPV

SEQ ID NO: 358 - protein, 9 aa, synthetic construct
YLKNVEEMV

SEQ ID NO: 359 - protein, 9 aa, synthetic construct
MLKEVEERV

SEQ ID NO: 360 - protein, 9 aa, synthetic construct
ALTDVEEAL

SEQ ID NO: 361 - protein, 9 aa, synthetic construct
LLKDVEEQV

SEQ ID NO: 362 - protein, 9 aa, synthetic construct
CLDDVEEKV

SEQ ID NO: 363 - protein, 9 aa, synthetic construct
RLKEIEEQV

SEQ ID NO: 364 - protein, 9 aa, synthetic construct
ALADIEERA

SEQ ID NO: 365 - protein, 9 aa, synthetic construct
GLKDIEEEL

SEQ ID NO: 366 - protein, 9 aa, synthetic construct
ALKDVDAKV

SEQ ID NO: 367 - protein, 9 aa, synthetic construct
TLKEIEEQV

SEQ ID NO: 368 - protein, 9 aa, synthetic construct
ELMDVEERV

SEQ ID NO: 369 - protein, 9 aa, synthetic construct
NLQDVEERV

SEQ ID NO: 370 - protein, 9 aa, synthetic construct
ELMDVEERV

SEQ ID NO: 371 - protein, 9 aa, synthetic construct
ALKDVEEAL

SEQ ID NO: 372 - protein, 9 aa, synthetic construct
FIKDVEERV

SEQ ID NO: 373 - protein, 9 aa, synthetic construct
ALNDVEKRV

SEQ ID NO: 374 - protein, 9 aa, synthetic construct
ALRDLEEKV

SEQ ID NO: 375 - protein, 9 aa, synthetic construct
GLKDLEERL

SEQ ID NO: 376 - protein, 9 aa, synthetic construct
ALKAVQERV

SEQ ID NO: 377 - protein, 9 aa, synthetic construct
ALKSVEERL

SEQ ID NO: 378 - protein, 9 aa, synthetic construct
YLKDVEERV

SEQ ID NO: 379 - protein, 9 aa, synthetic construct
FLKSYEEHL

SEQ ID NO: 380 - protein, 9 aa, synthetic construct
ALDDVEERV

SEQ ID NO: 381 - protein, 9 aa, synthetic construct
ALSNVVERL

SEQ ID NO: 382 - protein, 9 aa, synthetic construct
SLKDVDESV

SEQ ID NO: 383 - protein, 9 aa, synthetic construct
ILKDIEERV

SEQ ID NO: 384 - protein, 9 aa, synthetic construct
ALKEVAMRV

SEQ ID NO: 385 - protein, 9 aa, synthetic construct
RLKELEEAV

SEQ ID NO: 386 - protein, 9 aa, synthetic construct
VINDVNSRV

SEQ ID NO: 387 - protein, 9 aa, synthetic construct
ALKSVEEKV

SEQ ID NO: 388 - protein, 9 aa, synthetic construct
RMIEKMFYV

SEQ ID NO: 389 - protein, 9 aa, synthetic construct
FMREKIFYV

SEQ ID NO: 390 - protein, 9 aa, synthetic construct
KLSEKIFYL

SEQ ID NO: 391 - protein, 9 aa, synthetic construct
ALGEKIFYV

SEQ ID NO: 392 - protein, 9 aa, synthetic construct
ALSEKIFYL

SEQ ID NO: 393 - protein, 9 aa, synthetic construct
GLSEKIFYL

SEQ ID NO: 394 - protein, 9 aa, synthetic construct
KMSESVFTI

SEQ ID NO: 395 - protein, 9 aa, synthetic construct
GLTEKIFYL

SEQ ID NO: 396 - protein, 9 aa, synthetic construct
YASEKMFYV

SEQ ID NO: 397 - protein, 9 aa, synthetic construct
SVNEKIFYV

SEQ ID NO: 398 - protein, 9 aa, synthetic construct
VTAETIFYV

SEQ ID NO: 399 - protein, 9 aa, synthetic construct
GVSEKIFYV

SEQ ID NO: 400 - protein, 9 aa, synthetic construct
ANSEKIFYV

SEQ ID NO: 401 - protein, 9 aa, synthetic construct
QLKEKLFYV

SEQ ID NO: 402 - protein, 9 aa, synthetic construct
QLKEKLFYV

SEQ ID NO: 403 - protein, 9 aa, synthetic construct
KAADKIFYV

SEQ ID NO: 404 - protein, 9 aa, synthetic construct
DATEKIFYV

SEQ ID NO: 405 - protein, 9 aa, synthetic construct
ELSDKIFYV

SEQ ID NO: 406 - protein, 9 aa, synthetic construct
KAAEKFFYV

SEQ ID NO: 407 - protein, 9 aa, synthetic construct
KAAEKFFYV

SEQ ID NO: 408 - protein, 9 aa, synthetic construct
STLEKIIYV

SEQ ID NO: 409 - protein, 9 aa, synthetic construct
KASDKIFYV

SEQ ID NO: 410 - protein, 9 aa, synthetic construct
KTAEKIFRV

SEQ ID NO: 411 - protein, 9 aa, synthetic construct
TTLEKIIYV

SEQ ID NO: 412 - protein, 9 aa, synthetic construct
VQSEKIFPI

SEQ ID NO: 413 - protein, 9 aa, synthetic construct
VQSEKIFPI

SEQ ID NO: 414 - protein, 9 aa, synthetic construct
IAGEKIFYV

SEQ ID NO: 415 - protein, 9 aa, synthetic construct
KANEKIYYV

SEQ ID NO: 416 - protein, 9 aa, synthetic construct
NAAERIFYV

SEQ ID NO: 417 - protein, 9 aa, synthetic construct
SASEKIYYV

SEQ ID NO: 418 - protein, 9 aa, synthetic construct
KASEAIFYL

SEQ ID NO: 419 - protein, 9 aa, synthetic construct
GSSEKIFYV

SEQ ID NO: 420 - protein, 9 aa, synthetic construct
TTAQKIFYV

SEQ ID NO: 421 - protein, 9 aa, synthetic construct
KAGDRIYYV

SEQ ID NO: 422 - protein, 9 aa, synthetic construct
GQSEKIFSA

SEQ ID NO: 423 - protein, 9 aa, synthetic construct
KTKEKVFYI

SEQ ID NO: 424 - protein, 9 aa, synthetic construct
YTSEKIFYV

SEQ ID NO: 425 - protein, 9 aa, synthetic construct
KLSEKNFYV

SEQ ID NO: 426 - protein, 9 aa, synthetic construct
KISEDVFYV

SEQ ID NO: 427 - protein, 9 aa, synthetic construct
VVSEDIFYV

SEQ ID NO: 428 - protein, 9 aa, synthetic construct
KAYEKIFYV

SEQ ID NO: 429 - protein, 9 aa, synthetic construct
KVTEKIFYV

SEQ ID NO: 430 - protein, 9 aa, synthetic construct
KTSEKVFYV

SEQ ID NO: 431 - protein, 9 aa, synthetic construct
KAAEKVFYV

SEQ ID NO: 432 - protein, 9 aa, synthetic construct
KTTEKIFYV

SEQ ID NO: 433 - protein, 9 aa, synthetic construct
KVSEKIFYL

SEQ ID NO: 434 - protein, 9 aa, synthetic construct
GASERIFYV

SEQ ID NO: 435 - protein, 9 aa, synthetic construct
VASEKIFFV

SEQ ID NO: 436 - protein, 9 aa, synthetic construct
KISEKIRYV

SEQ ID NO: 437 - protein, 9 aa, synthetic construct
RASEKLYFV

SEQ ID NO: 438 - protein, 9 aa, synthetic construct
VASEKTFYV

SEQ ID NO: 439 - protein, 9 aa, synthetic construct
NYTNFCRFF

SEQ ID NO: 440 - protein, 9 aa, synthetic construct
GYYNFYRNF

SEQ ID NO: 441 - protein, 9 aa, synthetic construct
RYNGFYRFL

SEQ ID NO: 442 - protein, 9 aa, synthetic construct
NYIRFYRFL

SEQ ID NO: 443 - protein, 9 aa, synthetic construct
NYNNFLQKI

SEQ ID NO: 444 - protein, 9 aa, synthetic construct
KYNNYYRFL

SEQ ID NO: 445 - protein, 9 aa, synthetic construct
NYNSFYSFL

SEQ ID NO: 446 - protein, 9 aa, synthetic construct
NYNNFYVFL

SEQ ID NO: 447 - protein, 9 aa, synthetic construct
KYNEFYRLV

SEQ ID NO: 448 - protein, 9 aa, synthetic construct
EYSNFYRAF

SEQ ID NO: 449 - protein, 9 aa, synthetic construct
KYNDFYRYL

SEQ ID NO: 450 - protein, 9 aa, synthetic construct
DYANFYRAF

SEQ ID NO: 451 - protein, 9 aa, synthetic construct
DYANFYRAF

SEQ ID NO: 452 - protein, 9 aa, synthetic construct
FYNNFYRNL

SEQ ID NO: 453 - protein, 9 aa, synthetic construct
SYGNFYRFM

SEQ ID NO: 454 - protein, 9 aa, synthetic construct
SYNNLYRYL

SEQ ID NO: 455 - protein, 9 aa, synthetic construct
HYTDIYRFL

SEQ ID NO: 456 - protein, 9 aa, synthetic construct
DYNAFYRSF

SEQ ID NO: 457 - protein, 9 aa, synthetic construct
DYSNFYRAF

SEQ ID NO: 458 - protein, 9 aa, synthetic construct
DYSNFYRAF

SEQ ID NO: 459 - protein, 9 aa, synthetic construct
NYNNFIRYL

SEQ ID NO: 460 - protein, 9 aa, synthetic construct
NYNNFVRYL

SEQ ID NO: 461 - protein, 9 aa, synthetic construct
NYDNFFRML

SEQ ID NO: 462 - protein, 9 aa, synthetic construct
LYNNFERFL

SEQ ID NO: 463 - protein, 9 aa, synthetic construct
AYDEFYRFL

SEQ ID NO: 464 - protein, 9 aa, synthetic construct
DYSNFNRFL

SEQ ID NO: 465 - protein, 9 aa, synthetic construct
TYNEFYRFY

SEQ ID NO: 466 - protein, 9 aa, synthetic construct
EYDDSYRFL

SEQ ID NO: 467 - protein, 9 aa, synthetic construct
DYDNFYRNL

SEQ ID NO: 468 - protein, 9 aa, synthetic construct
MYANFYRFL

SEQ ID NO: 469 - protein, 9 aa, synthetic construct
TYNNFYRFF

SEQ ID NO: 470 - protein, 9 aa, synthetic construct
RYGNFYRFL

SEQ ID NO: 471 - protein, 9 aa, synthetic construct
NYNSFYRFF

SEQ ID NO: 472 - protein, 9 aa, synthetic construct
HYQNFYEFL

SEQ ID NO: 473 - protein, 9 aa, synthetic construct
KYDNYYRLF

SEQ ID NO: 474 - protein, 9 aa, synthetic construct
VYNNFYRLL

SEQ ID NO: 475 - protein, 9 aa, synthetic construct
NYNNFSIEF

SEQ ID NO: 476 - protein, 9 aa, synthetic construct
NYNNFSIEF

SEQ ID NO: 477 - protein, 9 aa, synthetic construct
NYNDFYDKF

SEQ ID NO: 478 - protein, 9 aa, synthetic construct
KYDNYYRLI

SEQ ID NO: 479 - protein, 9 aa, synthetic construct
NYSNIYRFL

SEQ ID NO: 480 - protein, 9 aa, synthetic construct
NYNNFYSFL

SEQ ID NO: 481 - protein, 9 aa, synthetic construct
NYNRFYDSI

SEQ ID NO: 482 - protein, 9 aa, synthetic construct
NYNNGYRTI

SEQ ID NO: 483 - protein, 9 aa, synthetic construct
DYNGFYANI

SEQ ID NO: 484 - protein, 9 aa, synthetic construct
HYDNLYSFL

SEQ ID NO: 485 - protein, 9 aa, synthetic construct
LYDNYYRIL

SEQ ID NO: 486 - protein, 9 aa, synthetic construct
YYDVFYNDL

SEQ ID NO: 487 - protein, 9 aa, synthetic construct
LYNTFYHFY

SEQ ID NO: 488 - protein, 9 aa, synthetic construct
MYMDLSDKI

SEQ ID NO: 489 - protein, 9 aa, synthetic construct
LYLFLSNKI

SEQ ID NO: 490 - protein, 9 aa, synthetic construct
YYLSISDKL

SEQ ID NO: 491 - protein, 9 aa, synthetic construct
CYLSLIDKI

SEQ ID NO: 492 - protein, 9 aa, synthetic construct
EYLSLTDQF

SEQ ID NO: 493 - protein, 9 aa, synthetic construct
KYLSLNDKI

SEQ ID NO: 494 - protein, 9 aa, synthetic construct
QYLTLTDKI

SEQ ID NO: 495 - protein, 9 aa, synthetic construct
EYLSMSTKI

SEQ ID NO: 496 - protein, 9 aa, synthetic construct
EYLSLSEKI

SEQ ID NO: 497 - protein, 9 aa, synthetic construct
KFLSLFDKI

SEQ ID NO: 498 - protein, 9 aa, synthetic construct
EYLSFSDKI

SEQ ID NO: 499 - protein, 9 aa, synthetic construct
KYLSLDDKI

SEQ ID NO: 500 - protein, 9 aa, synthetic construct
KYLSLDDKI

SEQ ID NO: 501 - protein, 9 aa, synthetic construct
EYLKLTDKI

SEQ ID NO: 502 - protein, 9 aa, synthetic construct
EYLSLSSKI

SEQ ID NO: 503 - protein, 9 aa, synthetic construct
EYLSLVDKI

SEQ ID NO: 504 - protein, 9 aa, synthetic construct
EYISLSDKI

SEQ ID NO: 505 - protein, 9 aa, synthetic construct
EYISLSDKI

SEQ ID NO: 506 - protein, 9 aa, synthetic construct
EYLSLSDSI

SEQ ID NO: 507 - protein, 9 aa, synthetic construct
EYLSLSDSI

SEQ ID NO: 508 - protein, 9 aa, synthetic construct
EYLSLSDKI

SEQ ID NO: 509 - protein, 9 aa, synthetic construct
GYLKLSDKI

SEQ ID NO: 510 - protein, 9 aa, synthetic construct
VYLSNSDKV

SEQ ID NO: 511 - protein, 9 aa, synthetic construct
EYLNINDKI

SEQ ID NO: 512 - protein, 9 aa, synthetic construct
EYLSLFDRM

SEQ ID NO: 513 - protein, 9 aa, synthetic construct
EYLTLGDKI

SEQ ID NO: 514 - protein, 9 aa, synthetic construct
EYLSLKDKL

SEQ ID NO: 515 - protein, 9 aa, synthetic construct
VYLSISEKI

SEQ ID NO: 516 - protein, 9 aa, synthetic construct
YYLELSEKI

SEQ ID NO: 517 - protein, 9 aa, synthetic construct
YYLALSDKI

SEQ ID NO: 518 - protein, 9 aa, synthetic construct
SYLSLANNI

SEQ ID NO: 519 - protein, 9 aa, synthetic construct
RYLSLSDKI

SEQ ID NO: 520 - protein, 9 aa, synthetic construct
IYLSLADKI

SEQ ID NO: 521 - protein, 9 aa, synthetic construct
RYLALSDKI

SEQ ID NO: 522 - protein, 9 aa, synthetic construct
KYLSLTDEI

SEQ ID NO: 523 - protein, 9 aa, synthetic construct
KYLSLADKI

SEQ ID NO: 524 - protein, 9 aa, synthetic construct
NYLSLSEKI

SEQ ID NO: 525 - protein, 9 aa, synthetic construct
QYLSLSNKI

SEQ ID NO: 526 - protein, 9 aa, synthetic construct
FYLSLNDKI

SEQ ID NO: 527 - protein, 9 aa, synthetic construct
EYLSLSQKI

SEQ ID NO: 528 - protein, 9 aa, synthetic construct
EYLSISNKI

SEQ ID NO: 529 - protein, 9 aa, synthetic construct
HYLSLSDKI

SEQ ID NO: 530 - protein, 9 aa, synthetic construct
FYLDLSDII

SEQ ID NO: 531 - protein, 9 aa, synthetic construct
EYLQLTTKI

SEQ ID NO: 532 - protein, 9 aa, synthetic construct
EYISLIDEI

SEQ ID NO: 533 - protein, 9 aa, synthetic construct
EYLWLADKI

SEQ ID NO: 534 - protein, 9 aa, synthetic construct
DYLSLSDIF

SEQ ID NO: 535 - protein, 9 aa, synthetic construct
EYLSISRII

SEQ ID NO: 536 - protein, 9 aa, synthetic construct
EYLRISDEI

SEQ ID NO: 537 - protein, 9 aa, synthetic construct
EYLSLKNII

SEQ ID NO: 538 - protein, 9 aa, synthetic construct
EYLSLVVEV

SEQ ID NO: 539 - protein, 9 aa, synthetic construct
AYLQLVFSI

SEQ ID NO: 540 - protein, 9 aa, synthetic construct
VYLQVVFGI

SEQ ID NO: 541 - protein, 9 aa, synthetic construct
SYLQKVFGI

SEQ ID NO: 542 - protein, 9 aa, synthetic construct
IYLVLVYGI

SEQ ID NO: 543 - protein, 9 aa, synthetic construct
EYMQLVAEI

SEQ ID NO: 544 - protein, 9 aa, synthetic construct
EYLQLIFPI

SEQ ID NO: 545 - protein, 9 aa, synthetic construct
QYLHIIFGI

SEQ ID NO: 546 - protein, 9 aa, synthetic construct
EYLQIVFNI

SEQ ID NO: 547 - protein, 9 aa, synthetic construct
EYLQLGFSI

SEQ ID NO: 548 - protein, 9 aa, synthetic construct
EYMQRVFGI

SEQ ID NO: 549 - protein, 9 aa, synthetic construct
EYLELLYGI

SEQ ID NO: 550 - protein, 9 aa, synthetic construct
VYLQLVRYV

SEQ ID NO: 551 - protein, 9 aa, synthetic construct
EYLQLVTYL

SEQ ID NO: 552 - protein, 9 aa, synthetic construct
AYLQLKYLY

SEQ ID NO: 553 - protein, 9 aa, synthetic construct
EYLQIVENV

SEQ ID NO: 554 - protein, 9 aa, synthetic construct
EYLQLVNQV

SEQ ID NO: 555 - protein, 9 aa, synthetic construct
EYLQLQRQL

SEQ ID NO: 556 - protein, 9 aa, synthetic construct
EYLELVFNY

SEQ ID NO: 557 - protein, 9 aa, synthetic construct
TYMQMVFGF

SEQ ID NO: 558 - protein, 9 aa, synthetic construct
RYLHLVFGI

SEQ ID NO: 559 - protein, 9 aa, synthetic construct
EYLQLFHQI

SEQ ID NO: 560 - protein, 9 aa, synthetic construct
EYAPLVFEI

SEQ ID NO: 561 - protein, 9 aa, synthetic construct
YYLQLVDKL

SEQ ID NO: 562 - protein, 9 aa, synthetic construct
LYLQLVFAF

SEQ ID NO: 563 - protein, 9 aa, synthetic construct
EYLQLVYGF

SEQ ID NO: 564 - protein, 9 aa, synthetic construct
KYLQTVFGL

SEQ ID NO: 565 - protein, 9 aa, synthetic construct
EYLQMAFGI

SEQ ID NO: 566 - protein, 9 aa, synthetic construct
EYLQLTTKI

SEQ ID NO: 567 - protein, 9 aa, synthetic construct
EYLQSAFGI

SEQ ID NO: 568 - protein, 9 aa, synthetic construct
EYLTLVCGF

SEQ ID NO: 569 - protein, 9 aa, synthetic construct
EYVQMIFSM

SEQ ID NO: 570 - protein, 9 aa, synthetic construct
AFLQLVETI

SEQ ID NO: 571 - protein, 9 aa, synthetic construct
EYLQQVIDI

SEQ ID NO: 572 - protein, 9 aa, synthetic construct
EYLSLVVEV

SEQ ID NO: 573 - protein, 9 aa, synthetic construct
EYLQAVREV

SEQ ID NO: 574 - protein, 9 aa, synthetic construct
EYLQLELRM

SEQ ID NO: 575 - protein, 9 aa, synthetic construct
EYLQDVFHY

SEQ ID NO: 576 - protein, 9 aa, synthetic construct
YYASLIHDI

SEQ ID NO: 577 - protein, 9 aa, synthetic construct
IYAAIIHDI

SEQ ID NO: 578 - protein, 9 aa, synthetic construct
LYQSEIHHI

SEQ ID NO: 579 - protein, 9 aa, synthetic construct
YYATLIHDI

SEQ ID NO: 580 - protein, 9 aa, synthetic construct
YYATLIHDI

SEQ ID NO: 581 - protein, 9 aa, synthetic construct
YYATLVHDI

SEQ ID NO: 582 - protein, 9 aa, synthetic construct
LYATAIHEI

SEQ ID NO: 583 - protein, 9 aa, synthetic construct
HYANVIHSW

SEQ ID NO: 584 - protein, 9 aa, synthetic construct
GYATVFHKI

SEQ ID NO: 585 - protein, 9 aa, synthetic construct
LYAAVLHEL

SEQ ID NO: 586 - protein, 9 aa, synthetic construct
LYASVLHDI

SEQ ID NO: 587 - protein, 9 aa, synthetic construct
LYATVFQDI

SEQ ID NO: 588 - protein, 9 aa, synthetic construct
LYATVFQDI

SEQ ID NO: 589 - protein, 9 aa, synthetic construct
LYFTVIHLL

SEQ ID NO: 590 - protein, 9 aa, synthetic construct
LYAAILHDI

SEQ ID NO: 591 - protein, 9 aa, synthetic construct
LYLTIIHDI

SEQ ID NO: 592 - protein, 9 aa, synthetic construct
LYATVIAEI

SEQ ID NO: 593 - protein, 9 aa, synthetic construct
AYGTVIHEI

SEQ ID NO: 594 - protein, 9 aa, synthetic construct
FYAAVLQDI

SEQ ID NO: 595 - protein, 9 aa, synthetic construct
LYSTVFQDI

SEQ ID NO: 596 - protein, 9 aa, synthetic construct
YYETVIHEI

SEQ ID NO: 597 - protein, 9 aa, synthetic construct
RYVTVIHDL

SEQ ID NO: 598 - protein, 9 aa, synthetic construct
AYQTVIFDI

SEQ ID NO: 599 - protein, 9 aa, synthetic construct
IYGAVINDI

SEQ ID NO: 600 - protein, 9 aa, synthetic construct
AYASVIQDI

SEQ ID NO: 601 - protein, 9 aa, synthetic construct
FYATVNHDI

SEQ ID NO: 602 - protein, 9 aa, synthetic construct
NYSAVIHDI

SEQ ID NO: 603 - protein, 9 aa, synthetic construct
LYVAVVQDI

SEQ ID NO: 604 - protein, 9 aa, synthetic construct
LYANVIVDI

SEQ ID NO: 605 - protein, 9 aa, synthetic construct
LYASVIVDI

SEQ ID NO: 606 - protein, 9 aa, synthetic construct
LYASVIVDI

SEQ ID NO: 607 - protein, 9 aa, synthetic construct
LYTTVISAI

SEQ ID NO: 608 - protein, 9 aa, synthetic construct
LYATVITDI

SEQ ID NO: 609 - protein, 9 aa, synthetic construct
PYVTVIHDL

SEQ ID NO: 610 - protein, 9 aa, synthetic construct
PYVTVIHDL

SEQ ID NO: 611 - protein, 9 aa, synthetic construct
EYAAAVHDI

SEQ ID NO: 612 - protein, 9 aa, synthetic construct
LYTTVIHDM

SEQ ID NO: 613 - protein, 9 aa, synthetic construct
AYATVISQM

SEQ ID NO: 614 - protein, 9 aa, synthetic construct
KYAAVIEEI

SEQ ID NO: 615 - protein, 9 aa, synthetic construct
LYSTVIHEI

SEQ ID NO: 616 - protein, 9 aa, synthetic construct
MYALSIHDI

SEQ ID NO: 617 - protein, 9 aa, synthetic construct
VYPLVIADI

SEQ ID NO: 618 - protein, 9 aa, synthetic construct
LYAKVIEDW

SEQ ID NO: 619 - protein, 9 aa, synthetic construct
PYATLIFDI

SEQ ID NO: 620 - protein, 9 aa, synthetic construct
LYAAVIWFL

SEQ ID NO: 621 - protein, 9 aa, synthetic construct
RYATVIGIL

SEQ ID NO: 622 - protein, 9 aa, synthetic construct
LYASLIHDL

SEQ ID NO: 623 - protein, 9 aa, synthetic construct
LYATLIMVL

SEQ ID NO: 624 - protein, 9 aa, synthetic construct
LYATLNHEL

SEQ ID NO: 625 - protein, 9 aa, synthetic construct
LYATVGFGL

SEQ ID NO: 626 - protein, 8 aa, synthetic construct
YTTVISDI

SEQ ID NO: 627 - protein, 9 aa, synthetic construct
LYATVYILV

SEQ ID NO: 628 - protein, 9 aa, synthetic construct
LYATVLVFL

SEQ ID NO: 629 - protein, 9 aa, synthetic construct
LYATVEQNL

SEQ ID NO: 630 - protein, 8 aa, synthetic construct
YDNVIHDI

SEQ ID NO: 631 - protein, 9 aa, synthetic construct
AYDNVIHDI